(19) United States
(12) Patent Application Publication
(10) Pub. No.: US 2016/0169890 A1
(43) Pub. Date: Jun. 16, 2016

(54) TRACKING DONOR-REACTIVE TCR AS A BIOMARKER IN TRANSPLANTATION

(71) Applicants: The Trustees of Columbia University in the City of New York, New York, NY (US); Adaptive Biotechnologies Corp., Seattle, WA (US)

(72) Inventors: Megan SYKES, Bronx, NY (US); Harlan ROBINS, Seattle, WA (US)

(21) Appl. No.: 14/892,512

(22) PCT Filed: Apr. 18, 2014
(86) PCT No.: PCT/US14/34685

§ 371 Date: Nov. 19, 2015

Related U.S. Application Data
(60) Provisional application No. 61/825,333, filed on May 20, 2013.

(51) Int. Cl. G01N 33/569; A61K 31/573; A61K 31/519; A61K 38/21; A61K 38/12; A61K 31/704; C07K 16/28; C12Q 1/68; A61K 31/52

(52) U.S. Cl. CPC G01N 33/56972; C12Q 1/6883; A61K 31/573; A61K 31/519; A61K 31/52; A61K 38/12; A61K 31/704; C07K 16/2809; C07K 16/2866; C07K 16/2887; A61K 38/217; A61K 38/215; A61K 38/212; G01N 2800/245

(57) ABSTRACT

The invention provides for the use of deep sequencing of the T-cell receptor beta CDR3 to identify, then track donor specific and/or recipient specific T cells in blood, urine and/or end-organs of transplant recipients.

### GOVERNMENT INTERESTS

This invention was made with government support under N01AI15416 awarded by the National Institutes of Health/Immune Tolerance Network. The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

The need for tolerance protocols in organ transplantation is underscored by the morbidity associated with chronic immunosuppressant use and the inability to prevent chronic rejection. Induction of donor chimerism is currently the most promising strategy to achieve renal allograft tolerance in humans. In a clinical trial, several combined kidney and bone marrow transplantation (CKBMT) recipients have tolerated their allograft for several years in the absence of any immunosuppressive medication, with development of donor-specific unresponsiveness in in-vitro assays. Donor chimerism was present for less than 3 weeks in each of these patients, and the precise mechanisms of tolerance are not known. Assessing deletional tolerance has previously been impossible due to the unavailability of markers for the many thousands of T cell clones responding to HLA alloantigens.

### SUMMARY OF THE INVENTION

The invention is directed to the use of deep sequencing of the T-cell receptor beta CDR3 to identify, then track the recipient anti-donor alloreactive T cell repertoire in blood and/or end-organs of transplant recipients. In one embodiment, the fate of these donor-reactive T cells can serve as a biomarker of rejection and/or tolerance.

An aspect of the invention encompasses the identification of biomarkers for determining whether a transplant has been rejected or whether there is tolerance. In one embodiment, the biomarkers are useful for monitoring whether a transplant has been rejected and/or whether there is tolerance in all types of transplants. In another embodiment, the transplant is an organ transplant (e.g., kidney, pancreas, liver, heart, lung, and intestine). In a further embodiment, the transplant is a cell transplant (e.g., stem cells, blood cells, islet cells). In specific embodiments, the transplant is a bone marrow transplant.

An aspect of the invention provides for a method of tracking particular T cells identified as donor-specific. In one embodiment, the biomarkers are useful for following rejection and/or tolerance in all types of transplants. In another embodiment, the transplant is an organ transplant (e.g., kidney, pancreas, liver, heart, lung, and intestine). In a further embodiment, the transplant is a cell transplant (e.g., stem cells, blood cells, islet cells). In specific embodiments, the transplant is a hematopoietic cell transplant.

An aspect of the invention provides for a method of treating organ transplant rejection in a subject in need thereof comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) administering an immunosuppressive therapy to the subject when the frequency of donor reactive T-cells in the post-transplant sample is equal to or higher than the frequency of T-cells determined in (b).

An aspect of the invention provides for a method of treating organ transplant rejection in a subject in need thereof comprising: (a) determining donor reactive T-cell receptor beta gene sequences in the subject; (b) determining the frequency of the donor reactive T-cell receptor beta gene sequences of (a) in a pre-transplant sample of the subject; (c) determining the frequency of donor reactive T-cell receptor beta gene sequences in a post-transplant sample from the subject; and (d) administering an immunosuppressive therapy to the subject when the frequency of donor reactive T-cell receptor beta gene sequences in the post-transplant sample is equal to or higher than the frequency of donor reactive T-cell receptor beta gene sequences in the pre-transplant sample.

An aspect of the invention provides for a method of treating graft versus host disease in an organ transplant recipient in need thereof comprising: (a) determining recipient reactive T-cell receptor beta gene sequences in a pre-transplant sample of an organ donor; (b) determining the frequency of donor T-cells in a post-transplant sample of the organ transplant recipient carrying the gene sequences in (a); and (c) administering an immunosuppressive therapy to the organ transplant recipient when recipient reactive donor T-cells are detected in the post-transplant sample.

An aspect of the invention provides for a method of diagnosing organ transplant rejection in a subject comprising: (a) determining donor reactive T-cell receptor beta gene sequences in the subject; (b) determining the frequency of donor reactive T-cells in a post-transplant sample of the subject carrying the gene sequences in (a); and (c) diagnosing organ transplant rejection in a subject when donor reactive T-cells are detected in the post-transplant sample.

An aspect of the invention provides for a method of reducing transplant rejection in a subject, wherein the subject has undergone an organ transplant, comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) administering an immunosuppressive therapy to the subject when the frequency of donor reactive T-cells in the post-transplant sample is equal to or higher than the frequency of T-cells determined in (b).

An aspect of the invention provides for a method of determining non-tolerance to an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) determining non-tolerance of the organ transplant when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of determining rejection of an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) determining rejection of the organ transplant when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of diagnosing organ transplant rejection in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) diagnosing organ transplant rejection in a subject when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of predicting organ transplant rejection in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) predicting the presence of organ transplant rejection in a subject when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of predicting organ transplant tolerance in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) predicting tolerance to an organ transplant in a subject when the frequency of donor reactive T-cells in the post-transplant sample is equal to or lower than the frequency of T-cells determined in (b). In one embodiment, the method further comprises reducing or withdrawing the administration of an immunosuppressive therapy to the subject.

An aspect of the invention provides for a method of identifying tolerance to an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) identifying tolerance to an organ transplant when the frequency of donor reactive T-cells in the post-transplant sample is equal to or lower than the frequency of T-cells determined in (b). In one embodiment, the method further comprises reducing or withdrawing the administration of an immunosuppressive therapy to the subject.

An aspect of the invention provides for a method of predicting organ transplant tolerance in a subject comprising: (a) determining donor reactive T-cell receptor beta gene sequences in the subject; (b) determining the frequency of T-cells carrying the gene sequences in (a) in a first post-transplant sample from the subject; (c) determining the frequency of T-cells carrying the gene sequences in (a) in a second post-transplant sample from the subject; and (d) predicting tolerance to an organ transplant in a subject when the frequency of T-cells determined in (c) is equal to or lower than the frequency of T-cells determined in (b). In one embodiment, the method further comprises reducing or withdrawing the administration of an immunosuppressive therapy to the subject.

An aspect of the invention provides for a method of predicting recipient tolerance in a subject comprising: (a) determining recipient reactive T-cell receptor beta gene sequences in a pre-transplant sample of an organ donor; (b) determining the frequency of donor T-cells in a post-transplant sample of the subject carrying the gene sequences in (a); and (c) predicting recipient tolerance in a subject when recipient reactive donor T-cells are not detected in the post-transplant sample. In one embodiment, the method further comprises reducing or withdrawing the administration of an immunosuppressive therapy to the subject.

An aspect of the invention provides for A method of predicting recipient non-tolerance in a subject comprising: (a) determining recipient reactive T-cell receptor beta gene sequences in a pre-transplant sample of an organ donor; (b) determining the frequency of donor T-cells in a post-transplant sample of the subject carrying the gene sequences in (a); and (c) predicting recipient non-tolerance in a subject when recipient reactive donor T-cells are detected in the post-transplant sample. In one embodiment, the method further comprises administering an immunosuppressive therapy to the subject.

In one embodiment, the method further comprises determining the frequency of donor reactive T-cells in a subsequent post-transplant sample from the subject. In another embodiment, the method further comprises comparing the frequency of donor reactive T-cells in the post-transplant sample to the frequency of donor reactive T-cells in the subsequent post-transplant sample. In another embodiment, the pre-transplant sample is collected before the subject undergoes an organ transplant with an organ from the organ donor. In one embodiment, the post-transplant sample is collected after the subject undergoes an organ transplant with an organ from an organ donor. In another embodiment, the post-transplant sample is blood. In another embodiment, the pre-transplant sample is blood. In a further embodiment, the post-transplant sample is urine. In another embodiment, the post-transplant sample is a tissue sample of the transplanted organ. In another embodiment, the T cell population further comprises CD4 T-cells. In another embodiment, the T cell population further comprises CD8 T-cells. In another embodiment the subject is a human. In another embodiment, the T-cell receptor beta gene sequences comprise T-cell receptor beta CDR3 gene sequences.

In one embodiment, the immunosuppressive therapy is a glucocorticoid, a cytostatic agent, an antibody, an immunophilin modulator, an interferon, plasmapheresis, or a combination thereof. In another embodiment, the glucocorticoid is methylprednisolone, corticosteroid, prednisone, prednisolone, dexamethasone, or betametasone. In a further embodiment, the cytostatic is methotrexate, azathioprine, mercaptopurine, dactinomycin, anthracyclines, mitomycin C, bleomycin, mithramycin, mycophenolate mofetil. In another embodiment, the antibody is a chimeric antibody, a humanized antibody, or a fully human antibody. In another embodiment, the antibody is thymoglobulin, Atgam, Muromonab-CD3, basiliximab, daclizumab, rituximab, or intravenous immunoglobulin. In another embodiment, the immunophilin modulator is cyclosporine, sirolimus, tacrolimus. In a further embodiment, the interferon is interferon alpha 2a, interferon alpha 2b, interferon beta 1a, interferon beta 1b, interferon gamma 1b. In another embodiment, the immunosuppressive therapy is methylprednisolone, corticosteroid, thymoglobulin, basiliximab, rituximab, intravenous immunoglobulin, tacrolimus, mycophenolate, plasmapheresis, or a combination thereof.

In one embodiment the organ transplant is a heart transplant, a kidney transplant, a liver transplant, a lung transplant, a pancreas transplant, an intestine transplant, a stomach transplant, a testis transplant, a thymus transplant, a hematopoietic cell transplant, or combination thereof. In another embodiment, the organ transplant is a hematopoietic celltransplant, a tendon transplant, a cornea transplant, a skin transplant, a heart valve transplant, a nerve transplant, a vein transplant, a bone transplant, an Islets of Langerhans transplant, or a combination thereof. In another embodiment, the organ transplant is a face transplant, a hand transplant, a leg transplant, a penis transplant, a uterus transplant, an ovary transplant, a hematopoietic cell transplant, or a combination thereof. In another embodiment, the organ transplant is a combined kidney and hematopoietic cell transplant. In another embodiment, the organ transplant is an intestine transplant. In another embodiment, the organ transplant is a kidney transplant. In another embodiment, the organ transplant is a hematopoietic cell transplant.

An aspect of the invention provides a method of identifying donor reactive T-cells in a subject comprising: (a) isolating peripheral blood mononuclear cells (PBMCs) from an organ donor; (b) isolating a first T-cell population from a pre-transplant sample of the subject; (c) isolating a second T-cell population, wherein the second T-cell population comprises T-cells from the first T-cell population that proliferate when cultured with the PBMCs from the organ donor; and (d) comparing the first T-cell population with the second T-cell population, wherein the donor reactive T-cells comprise T-cells with a frequency of at least about 0.01% in the second T-cell population and about 5-fold higher frequency in the second T-cell population compared to the first T-cell population.

An aspect of the invention provides A method of identifying recipient reactive T-cells comprising: (a) isolating a first T-cell population from a sample from an organ donor; (b) isolating peripheral blood mononuclear cells (PBMCs) from a pre-transplant sample of a subject, wherein the subject is a recipient of an organ transplant from the organ donor; (c) isolating a second T-cell population, wherein the second T-cell population comprises T-cells from the first T-cell population that proliferate when cultured with the PBMCs from (b); and (d) comparing the first T-cell population with the second T-cell population, wherein the recipient reactive T-cells comprise T-cells with a frequency of at least about 0.01% in the second T-cell population and about 5-fold higher frequency in the second T-cell population compared to the first T-cell population.

In one embodiment, the organ is a heart, a kidney, a liver, a lung or lungs, a pancreas, an intestine, a stomach, a testis, a thymus, hematopoietic cells, or a combination thereof. In another embodiment, the pre-transplant sample is collected before the subject undergoes an organ transplant with an organ from the organ donor. In another embodiment, the method further comprising isolating DNA from the first T-cell population. In another embodiment, the method further comprises isolating DNA from the second T-cell population.

In one embodiment the method further comprises determining T-cell receptor beta gene sequences of the first T-cell population by sequencing the complementarity determining region 3 (CDR3) region of the T-cell receptor beta gene of the first T-cell population. In another embodiment, the method further comprises determining T-cell receptor beta gene sequences of the second T-cell population by sequencing the complementarity determining region 3 (CDR3) region of the T-cell receptor beta gene of the second T-cell population.

An aspect of the invention provides a method of identifying donor reactive T-cells in a subject comprising: (a) isolating peripheral blood mononuclear cells (PBMCs) from an organ donor; (b) isolating a first T-cell population from a pre-transplant sample of the subject; (c) isolating a second T-cell population, wherein the second T-cell population comprises T-cells from the first T-cell population that proliferate when cultured with the PBMCs from the organ donor; (d) isolating DNA from the first T-cell population; (e) determining T-cell receptor beta gene sequences of the first T-cell population by sequencing the complementarity determining region 3 (CDR3) region of the T-cell receptor beta gene of the isolated DNA from the first T-cell population; (f) isolating DNA from the second T-cell population; (g) determining T-cell receptor beta gene sequences of the second T-cell population by sequencing the CDR3 region of the T-cell receptor beta gene of the isolated DNA from the second T-cell population; and (h) comparing the T-cell receptor beta gene sequences of the first T-cell population with the T-cell receptor beta gene sequences of the second T-cell population, wherein the donor reactive T-cells comprise T cells with T-cell receptor beta gene sequences with a frequency of at least about 0.01% in the second T-cell population and about 5-fold higher frequency in the second T-cell population compared to the first T-cell population.

In one embodiment the sample is blood. In another embodiment, the first and second T cell populations further comprise CD4 T-cells. In another embodiment, the first and second T cell populations further comprise CD8 T-cells. In a further embodiment, the subject is a human.

An aspect of the invention provides a kit for determining donor reactive T-cell frequency in a post-transplant sample comprising: (a) a sample container; and (b) donor reactive T-cell receptor beta gene sequences, wherein each donor reactive T-cell receptor beta gene sequence is determined from T-cells from the recipient that proliferate in response to PBMCs from an organ donor.

An aspect of the invention provides a kit for determining recipient reactive T-cell frequency in a post-transplant sample comprising: (a) a sample container; and (b) recipient reactive T-cell receptor beta gene sequences, wherein each recipient reactive T-cell receptor beta gene sequence is determined from T-cells from the donor that proliferate in response to PBMCs from an organ transplant recipient.

In one embodiment, the kit further comprises an antibody to CD4. In one embodiment, the kit further comprises an antibody to CD8.

An aspect of the invention provides a kit comprising: (a) a urine sample container where DNA in the urine sample is preserved; and (b) donor reactive T-cell receptor beta gene sequences, wherein each donor reactive T-cell receptor beta gene sequence is determined from T-cells from the recipient that proliferate in response to PBMCs from an organ donor.

An aspect of the invention provides a kit comprising: (a) a urine sample container where DNA in the urine sample is preserved; (b) recipient reactive T-cell receptor beta gene sequences, wherein each recipient reactive T-cell receptor beta gene sequence is determined from T-cells from the donor that proliferate in response to PBMCs from an organ transplant recipient.

### BRIEF DESCRIPTION OF THE FIGURES

FIG. 1 is a schematic showing the regions recognized by primers for deep sequencing of TCRB CDR3 regions to identify and track alloreactive T cells. High-throughput sequencing of CDR3 regions permits estimation of relative frequency of each CDR3 sequence in the T-cell population. It can reliably detect clones at a level of 1 in 100,000 (0.001%).

FIG. 2 is a schematic of the experimental design for isolating donor-reactive T cells via Mixed-Lymphocyte Reaction (MLR).

FIGS. 3A-B show graphs of the reproducible clonal expansions in repeated MLRs on the same responder-stimulator pair. The most frequently detected 30 CDR3 region TCR 0 chain sequences for divided CD4 (FIG. 3A) and CD8 (FIG. 3B) T cells in mixed lymphocyte reaction (MLR) assays against the same stimulator from separate blood draws at T1 and T2 (MLR1 and MLR2, respectively). Most of the clones showing marked expansions in the first MLR (compared to unstimulated cells) were also expanded in the second MLR. Each line represents a single CDR3 sequence. The Y-axis respresents the frequency.

FIG. 4 shows representative CDR3 sequencing results (e.g., the frequencies of CD8 T cells) in ITN Patient 6. The figures also shows representative CDR3 sequencing results from a tolerant CKBMT patient: CD4+ T-cells (FIG. 4 continued on subsequent page).

FIG. 5 shows a validation study comparing the top clones between pre-transplant stimulated and unstimulated conditions. All clones with a frequency >10̂−4 in pre-transplant stimulated or unstimulated conditions were considered. A frequency of 10̂−5 was the cut-off if present after 1.5 year.

FIG. 6 is a schematic of the T cell receptor (variable (V) and constant (C) regions; top) and the CDR3region (bottom).

FIG. 7 shows the gating strategy for cell sorting of day 6 CFSE-MLR cultures. CD3+Violet− cells, representing the responder T cells, were selected and further separated into CD4+ and CD8+ subgroups. Within each subgroup, the CFSE-low cells were isolated for DNA extraction.

FIGS. 8A-C show the results of the validation study. FIG. 8A: Overlap of the nucleotide sequences of the top 100 alloreactive clones from two parallel MLRs (Timepoint 1 and Timepoint 2). 64% of the CD4+ clones and 22% of the CD8+ clones in each “top 100” subset had identical nucleotide sequences. FIG. 8B: Summed frequencies of the top 100 CD4 and CD8 alloreactive clones in MLR at Timepoint 1 and Timepoint 2. The top 100 clones comprise a much higher proportion of the stimulated (alloreactive) repertoire as compared to the unstimulated repertoire, consistent with allo-specific expansion. FIG. 8C: The alloreactive clones that are detected at Timepoine 1 and Timepoint 2 display a linear relationship when their frequencies are plotted against each other on a logarithmic scale (r=0.7 CD4, p<0.001; r=0.8 CD8, p<0.001), demonstrating a similar degree of expansion in each MLR. No such linear relationship is observed when the alloreactive clone frequencies in the stimulated population are plotted against the frequencies in the unstimulated population (r=0.3 CD4, p<0.001, r=0.2 CD8, p<0.001).

FIGS. 9A-C show Summary of clonal analysis results for all six patients. FIGS. 9A and 9B: Anti-donor clones defined as nucleotide clones with ≧10-4 frequency that are expanded at least 5-fold relative to their frequency in the pre-transplant unstimulated sample. “Presence” defined as a frequency of 10-5 in Subject 1, Subject 2, Subject 4, and IS#1, 5×10-5 in Subject 5 and IS#2 for all unstimulated populations. Fold change is the odds ratio of an increase or decrease in donor-reactive clones relative to pre-transplant (pre-transplant=1). Red point=statistically significant reduction or increase (P-value<0.05). FIG. 9C: Volcano plot summary of clonal analysis. “Tolerant” refers to ITN Subjects 1, 2, and 4. “Non-tolerant” refers to ITN Subject 5 and both traditional kidney transplant recipients (IS#1 and IS#2). The horizontal dotted line denotes a P-value of 0.05; dots falling above this line are statistically significant. The vertical dotted line represents an odds ratio of one; dots falling to the left of this line represent a reduction in donor-reactive clones relative to pre-transplant and dots to the right represent an expansion of donor-reactive clones relative to pre-transplant.

FIGS. 10A-D show the MLR for Subjects 1, 2, 4, and 5.

FIGS. 11A-D show the CML for Subjects 1 and 2 (FIGS. 11A-B) and Subject 4 and 5 (FIGS. 11C-D). Anti-donor and anti-3^{rd} party responses are shown.

FIGS. 12A-C show TCR repertoire turnover in kidney allograft recipients. FIG. 12A: Jensen-Shannon Divergence (JSD) of pre-transplantation and post-transplantation (last post-transplantation time point) TCR repertoires. Jensen-Shannon divergence (JSD) on top 1000 nucleotide clones (JSD=0, repertoires identical; JSD=1, complete repertoire divergence). HC=healthy controls: average of four healthy controls in whom TCR sequencing was performed over a 1-year interval; JSD on top 1000 nucleotide clones. FIG. 12B: Anti-donor clonal analysis relative to repertoire turnover. Detection of clones in post-transplant samples is compared to “background” detection in pre-transplant unstimulated sample; “presence” threshold=frequency>10-6. Fold change is the odds ratio of an increase or decrease in anti-donor clones relative to pre-transplant (pre-transplant=1). Red point=statistically significant reduction or increase (P-value>0.05). FIG. 12C: CD4 T cell repertoire clonality over time post-transplantation (clonality=0, all clones unique; clonality=1, all clones identical). “Tolerant” refers to Subjects 1, 2, and 4. “Non-tolerant” refers to ITN Subject 5 and both traditional kidney transplant recipients (IS#1 and IS#2).

FIGS. 13A-B shows validation study setup and cell counts. FIG. 13A: Two CFSE MLRs were set up in parallel, all of which contained responder PBMC from the same healthy control subject but obtained at two different timepoints (Timepoint 2 contained PBMC obtained from a blood draw performed two weeks after Timepoint 1; MLRs contained irradiated stimulator PBMC from the same healthy volunteer.) FIG. 13B: Clonal expansions of CD4 and CD8 T cells in response to alloantigens. The data are corrected for erroneous reads, for which the average error rate is about 0.25% in typical Illumina HiSeq runs.

FIG. 14 shows cell numbers, number of unique clones, and total number of reads for each patient sample HLA mismatches: Subject 1: 2/4 Class I, 2/4 Class II; Subject 2: 1/4 Class I, 0/4 Class II. Subject 4: 2/4 Class 1, 0/4 class II; Subject 5: 2/4 Class I; 0/4 Class II, IS#1: 2/4 Class I, 2/4 Class II; IS#2: 2/4 Class I, 4/4 Class II.

FIGS. 15A-D shows clonal analysis in CKBMT subjects. FIG. 15A: Subject 1: Donor-reactive clones are defined as clones with ≧10-4 frequency that are expanded at least 5-fold relative to their frequency in the pre-transplant unstimulated sample. “Presence” defined as a frequency of 10-5 in all unstimulated populations; *P<0.05 as compared to pre-transplant. FIG. 13B: Subject 2: Donor-reactive clones defined as clones with ≧10-4 frequency that are expanded at least 5-fold relative to their frequency in the pre-transplant unstimulated sample. “Presence” defined as a frequency of 10-5 in all unstimulated populations. *P<0.05 as compared to pre-transplant FIG. 13C: Subject 4: Donor-reactive clones defined as clones with ≧10-4 frequency that are expanded at least 5-fold relative to their frequency in the pre-transplant unstimulated sample. “Presence” defined as a frequency of 10-5 in all unstimulated populations. *P<0.05 as compared to pre-transplant. FIG. 13D: Subject 5: Donor-reactive clones defined as clones with ≧10-4 frequency that are expanded at least 5-fold relative to their frequency in the pre-transplant unstimulated sample. “Presence” defined as a frequency of 5×10-5 in all unstimulated populations. Changes relative to pre-transplant did not reach statistical significance at 6 months or at 1 year.

FIGS. 16A-F shows anti-donor clonal analysis at increasing fold-expansion criteria. Odds ratio of an increase or decrease in donor-reactive clones as compared to pre-transplant is plotted against increasing fold-expansion criteria as compared to an unstimulated pre-transplantation sample (3-fold, 5-fold, 7-fold, and 10-fold). Red points are statistically significant (p<0.05).

FIGS. 17A-D show limiting dilution analysis (CTLp (FIGS. 17A-B) and HTL (FIGS. 17C-D)) for Subject 1.

FIGS. 18A-D show limiting dilution analysis (CTLp (FIGS. 18A-B) and HTL (FIGS. 18C-D)) for Subject 2.

FIGS. 19A-D show limiting dilution analysis (CTLp (FIGS. 19A-B) and HTL (FIGS. 19C-D)) for Subject 4.

FIGS. 20A-B. FIG. 20A: Log scatter plots comparing the T cells that expand in an MLR in response to donor stimulation pre-transplantation and 1-year post-transplantation (log read counts, amino acid CDR3+V&J families). CD4: r=0.26; CD8: r=0.28. FIG. 20B: Log scatter plots comparing the T cells that expand in an MLR in response to donor pre-transplantation and a third party pre-transplantation (log read counts, amino acid CDR3+V&J families). CD4: r=0.24; CD8: r=0.12. Sequences in both pre-transplant and post-transplant MLR: CD4=178; CD8=114). Sequences in both pre-transplant anti-donor and anti-third party MLRs: CD4=418; CD8=271).

FIG. 21A-D show limiting dilution analysis (CTLp (FIGS. 21A-B) and HTL (FIGS. 21C-D)) for Subject 5.

FIGS. 22A-B show clonal analysis in patients is#1 (non-rejector) and is#2 (rejector). FIG. 22A: IS#1. Donor-reactive clones defined as clones with ≧10-4 frequency that are expanded at least 5-fold relative to their frequency in the pre-transplant unstimulated sample. “Presence” defined as a frequency of 10-5 in all unstimulated populations. *P<0.05 as compared to pre-transplant. FIG. 22B: IS#2. Donor-reactive clones defined as clones with ≧10-4 frequency that are expanded at least 5-fold relative to their frequency in the pre-transplant unstimulated sample. “Presence” defined as a frequency of 5×10-5 in unstimulated populations. *P<0.05 as compared to pre-transplant.

FIGS. 23A-E show results of anti-third-party clonal analysis. Anti-third party clones defined as clones (amino acid CDR3+V/J families) with ≧10-4 frequency that are expanded at least 5-fold relative to their frequency in the pre-transplant unstimulated sample. “Presence” defined as a frequency of 10-5 in ITN 1, ITN 2, and ITN 4 and 5×10-5 for ITN 5 for all unstimulated populations. *=p<0.05 (significant decrease); ⋄=p<0.05 (significant increase).

FIG. 24 shows anti-donor clonal analysis relative to repertoire turnover at increasing fold-expansion criteria. Odds ratio of an increase or decrease in donor-reactive clones relative to repertoire turnover is plotted against increasing fold-expansion criteria (fold, 5-fold, 7-fold, and 10-fold). Red points are statistically significant (p<0.05).

FIG. 25 shows alloreactice clones tracking in one ITx patient who experienced skin GVHD and subsequently intestinal transplant rejection.

FIGS. 26A-C shows identification and monitoring of GvH and HvG-specific T cell clones with high throughput sequencing. FIG. 26C shows the sorting purity.

FIGS. 27A-B shows presence of GvH clones in the gut, blood and skin at the time of GVHD.

FIGS. 28A-B shows post-Tx trajectories of pre-Tx GvH clones for CD8 and CD4.

FIG. 29 shows the GvH clones expanded significantly more than unstim clones at the time of GVHD.

FIGS. 30A-B shows Presence of HvG clones in the blood at the time of the rejection.

FIGS. 31A-C shows presence of HvG CD4+ clones among IEL at the time of the rejection. FIG. 31A: HvG and non-donor-reactive clones were tracked in IEL recipient-derived cell lines expanded from a biopsy performed on POD62 (rejection).

FIGS. 32A-B shows presence of HvG CD8+ clones in the blood at the time of the rejection.

FIGS. 33A-C shows Presence of HvG CD8+ clones among IEL at the time of the rejection. FIG. 33A: HvG and non-donor-reactive clones were tracked in IEL recipient-derived cell lines expanded from a biopsy performed on POD62 (rejection).

### DETAILED DESCRIPTION OF THE INVENTION

### Abbreviations and Definitions

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The invention is directed to the use of deep sequencing of the T-cell receptor beta CDR3 to identify, then track the recipient anti-donor alloreactive T cell repertoire in blood and/or end-organs of transplant recipients. In one embodiment, the fate of these donor-reactive T cells can serve as a biomarker of rejection and/or tolerance.

As would be apparent to one of ordinary skill in the art, any method or composition described herein can be implemented with respect to any other method or composition described herein.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The invention is directed to identification of biomarkers for transplant rejection and tolerance via use of high throughput CDR3 genomic sequencing of a CKBMT (combined kidney and bone marrow transplantation) recipient's donor-reactive T cells prior to transplant. Such high throughput CDR3 sequencing can allow for identification of the thousands of TCR clones that specifically recognize his/her organ donor's alloantigens. In one embodiment, the invention is directed to a method of tracking donor-reactive T cells in the post-transplant period.

Tolerance protocols are needed in transplantation. Despite progress in short-term graft and patient survival after kidney transplant, chronic allograft loss remains a persistent problem. Immunosuppressive medications are associated with significant morbidity.

Induction of donor chimerism is currently the most promising strategy to achieve renal allograft tolerance in humans. Chimerism is a state in which all lymphoid and hematopoietic elements are comprised of a mixture of host and donor types. A non-myeloablative conditioning regimen allows donor bone marrow to engraft in the kidney transplant recipient. The presence of donor-derived dendritic cells within the recipient thymus leads to negative selection of donor-reactive T cells. In a clinical trial, several CKBMT recipients have tolerated their allograft for several years in the absence of any immunosuppressive medication. Donor chimerism was present for less than 3 weeks in each of these patients.

Donor chimerism is transient. The mechanism of long term tolerance still needs to be determined. Mechanisms of peripheral tolerance include deletion, anergy (a state in which a T cell is refractory to activation by a specific antigen), and regulation/suppression by regulatory T cells. While some lines of evidence support a suppressive mechanism, in vitro studies also support a role for anergy or deletion of alloreactive T cells at later time points post-transplant. Assessing deletional tolerance has previously been impossible due to the unavailability of markers for the many thousands of T cell clones responding to HLA alloantigens.

The Challenge of Tracking Alloreactive T Cells

In-vitro assays showing unresponsiveness and not implicating suppression cannot distinguish between anergy and deletion. Allo MHC-reactive T-cell repertoire is extremely broad: 1-10% of T cells respond to a given donor. Minor HA-reactive T cells can be tracked with tetramers, but the likelihood that thousands of peptide/MHC specificities are recognized makes tetramers unlikely to be useful for tracking of alloreactivity in HLA-mismatched transplants.

In patients receiving combined kidney and bone marrow transplantation (CKBMT)), in vitro assays have shown a complete (in NKD03 and some ITN036ST patients) or partial (other ITN036ST patients) loss of anti-donor alloreactivity, with recovery of anti-third party responses, in several assays, including mixed lymphocyte reaction (MLR) proliferative and cell-mediated lympholysis (CML) cytotoxicity assays and limiting dilution assays (LDA). The pattern of LDA curves is not suggestive of a suppressive mechanism of tolerance and depletion of Tregs does not reveal anti-donor reactivity in the late (>1 year) post-transplant assays. Together, the data are consistent with a deletional mechanism of tolerance, but there has never been a way to definitively distinguish deletion from anergy.

Since responses to allogeneic MHC molecules are highly polyclonal, involving 1-7% of the T cell repertoire, a very large number of clones would be predicted to be involved in this response. High throughput CDR3 sequencing of T cells responding to a given allogeneic donor can reveal expansion of thousands of alloreactive TCR sequences and that identification of the expanded alloreactive TCRs in an MLR can provide a footprint of the response of a recipient to his/her organ donor, allowing tracking of these T cells in vivo. Thus, high throughput CDR3 sequencing of a CKBMT recipient's donor-reactive T cells prior to transplant would allow identification of thousands of TCR clones that recognize their organ donor's alloantigens. These donor-reactive T cells can then tracked in vivo in the post-transplant period.

In one embodiment, alloreactive T cells can be identified using new-generation high throughput sequencing of CDR3 regions. This technique involves TCRβ CDR3 amplification with primers specific for all 54 expressed Vβ and all 13 Jβ regions adapted for solid phase PCR on the Ilumina HiSeq, allowing high throughput sequencing of millions of T cell clones as well as detection of rare clones. Analysis of genomic DNA allows unbiased sequencing of αβ T cells. This technique has been used by the inventors to identify alloreactive CD4 and CD8 αβ T cell populations in MLRs. Reproducibility of the expansion of alloreactive clones has been demonstrated in an MLR between the same donors. In the first ITN036ST patient, marked reductions have been demonstrated in donor-reactive clones in PBMC at 6 months and 1.5 years post-transplant. Of the clones that were expanded, at least 10× in MLR, only 221164 (13.4%) CD4+ and 10/64 (15.6%) CD8+ clones were still detectable in blood at 1.5 years post-transplant. This reduction is highly specific for the donor-reactive clones (p<10⁻¹⁵).

Significance

For the first time, this strategy developed allows identification of donor-reactive T cells and physical assessment of their fate following transplantation. The invention involves the use of this approach to obtain a biomarker for rejection and tolerance in transplant patients. Although there are are “tolerance signatures” based on gene expression microarrays, these other technologies do not specifically look at the T cells recognizing donor antigents.

Methods of Identifying T-Cell Receptor Beta Gene Sequences

An aspect of the invention provides a method of identifying donor reactive T-cells in a subject comprising: (a) isolating peripheral blood mononuclear cells (PBMCs) from an organ donor; (b) isolating a first T-cell population from a pre-transplant sample of the subject; (c) isolating a second T-cell population, wherein the second T-cell population comprises T-cells from the first T-cell population that proliferate when cultured with the PBMCs from the organ donor; and (d) comparing the first T-cell population with the second T-cell population, wherein the donor reactive T-cells comprise T-cells with a frequency of at least about 0.01% in the second T-cell population and about 5-fold higher frequency in the second T-cell population compared to the first T-cell population.

An aspect of the invention provides a method of identifying recipient reactive T-cells comprising: (a) isolating a first T-cell population from a sample from an organ donor; (b) isolating peripheral blood mononuclear cells (PBMCs) from a pre-transplant sample of a subject, wherein the subject is a recipient of an organ transplant from the organ donor; (c) isolating a second T-cell population, wherein the second T-cell population comprises T-cells from the first T-cell population that proliferate when cultured with the PBMCs from (b); and (d) comparing the first T-cell population with the second T-cell population, wherein the recipient reactive T-cells comprise T-cells with a frequency of at least about 0.01% in the second T-cell population and about 5-fold higher frequency in the second T-cell population compared to the first T-cell population.

In one embodiment, the organ is a heart, a kidney, a liver, a lung or lungs, a pancreas, an intestine, a stomach, a testis, a thymus, hematopoietic cells, or a combination thereof. In another embodiment, the pre-transplant sample is collected before the subject undergoes an organ transplant with an organ from the organ donor. In another embodiment, the method further comprising isolating DNA from the first T-cell population. In another embodiment, the method further comprises isolating DNA from the second T-cell population.

In one embodiment the method further comprises determining T-cell receptor beta gene sequences of the first T-cell population by sequencing the complementarity determining region 3 (CDR3) region of the T-cell receptor beta gene of the first T-cell population. In another embodiment, the method further comprises determining T-cell receptor beta gene sequences of the second T-cell population by sequencing the complementarity determining region 3 (CDR3) region of the T-cell receptor beta gene of the second T-cell population.

An aspect of the invention provides a method of identifying donor reactive T-cells in a subject comprising: (a) isolating peripheral blood mononuclear cells (PBMCs) from an organ donor; (b) isolating a first T-cell population from a pre-transplant sample of the subject; (c) isolating a second T-cell population, wherein the second T-cell population comprises T-cells from the first T-cell population that proliferate when cultured with the PBMCs from the organ donor; (d) isolating DNA from the first T-cell population; (e) determining T-cell receptor beta gene sequences of the first T-cell population by sequencing the complementarity determining region 3 (CDR3) region of the T-cell receptor beta gene of the isolated DNA from the first T-cell population; (f) isolating DNA from the second T-cell population; (g) determining T-cell receptor beta gene sequences of the second T-cell population by sequencing the CDR3 region of the T-cell receptor beta gene of the isolated DNA from the second T-cell population; and (h) comparing the T-cell receptor beta gene sequences of the first T-cell population with the T-cell receptor beta gene sequences of the second T-cell population, wherein the donor reactive T-cells comprise T cells with T-cell receptor beta gene sequences with a frequency of at least about 0.01% in the second T-cell population and about 5-fold higher frequency in the second T-cell population compared to the first T-cell population.

In one embodiment the sample is blood. In another embodiment, the first and second T cell populations further comprise CD4 T-cells. In another embodiment, the first and second T cell populations further comprise CD8 T-cells. In a further embodiment, the subject is a human.

Cells of the Invention

Peripheral blood mononuclear cells (also referred to throughout as “PBMCs”) are a heterogeneous population of blood cells that includes, but is not limited to, lymphocytes, monocytes, macrophages and dendritic cells. A lymphocyte is a type of while blood cell that includes T cells, B cells and NK cells. PMBCs may or may not express surface markers. In one embodiment, PBMCs are isolated from an organ donor. In another embodiment, PBMCs are isolated from an organ transplant recipient. Characteristics of PBMCs will be known to one of skill in the art, for further information the reader is referred to Lindau D. et al. 2013 Immunology 138(2):105-115.

PBMCs can be isolated from a subject by methods known in the art. For example, peripheral whole blood can be drawn from a subject. The whole blood can be separated by density gradient centrifugation using Ficoll into more dense erythrocytes (red blood cells) and polymorphonuclear cells (PMNs) and less dense PBMCs. In another embodiment, PBMCs can be isolated from a tissue biopsy.

In one embodiment, T cells can express a surface marker. In another embodiment, T cells do not express a surface marker. In one embodiment, T cells express the surface marker CD3. In another embodiment, T cells do not express the surface marker CD3. In another embodiment, T cells express the surface marker CD4. In another embodiment, T cells do not express the surface marker CD4. In a further embodiment, T cells express the surface marker CD8. In another embodiment, T cells do not express the surface marker CD8.

Samples used in the methods of the present invention can include, for example, a bodily fluid from a subject, including, blood and blood plasma, lymph, mucus (including snot and phlegm), saliva, serum, urine, feces, internal body fluids, including cerebrospinal fluid surrounding the brain and the spinal cord. In one embodiment, the sample is a blood sample. The blood sample can be about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mL. In another embodiment, the sample is urine. The urine sample can be about 1, 2, 3, 4, 5, 10, 25, 50, 100, 200, or 500 mL. In one embodiment, the T-cells carrying the donor-specific non-tolerant T-cell receptor beta gene sequences can be identified from the blood followed by later monitoring the levels of non-tolerant T-cells from a different body fluid, for example, urine. Samples can be obtained from a subject by methods known in the art.

Samples used in the methods of the present invention can also be a biopsy. In one embodiment, the biopsy can be from, for example, brain, liver, lung, heart, colon, kidney, or bone marrow. In another embodiment, the biopsy can be from the organ that has been transplanted. Any biopsy technique used by those skilled in the art can be used for isolating a sample from a subject. For example, a biopsy can include, but is not limited to, an open biopsy, a closed biopsy, a core or incisional biopsy, an excisional biopsy, and a fine needle aspiration biopsy.

In one embodiment, the sample includes immune cells, PBMCs, and/or lymphocytes. In another embodiment, the sample includes T-cells and/or B-cells. T-cells (T lymphocytes) include, for example, cells that express T cell receptors. T-cells include Helper T cells (effector T cells or Th cells), cytotoxic T cells (CTLs), memory T cells, and regulatory T cells. In one embodiment, the sample includes nucleic acid, for example, DNA (e.g., genomic DNA or mitochondrial DNA) or RNA (e.g., messenger RNA or microRNA).

Nucleic Acids

As used herein, “TCRβ molecule” refers to a T-cell receptor beta nucleic acid (including, for example, genomic DNA, complementary DNA (cDNA), synthetic DNA, as well as any form of corresponding RNA) which encodes a polypeptide corresponding to a T cell receptor beta protein, or fragment thereof. A “TCRβ molecule” can also refer to a T cell receptor beta protein, or a fragment thereof.

The nucleic acid can be any type of nucleic acid, including genomic DNA, complementary DNA (cDNA), synthetic or semi-synthetic DNA, as well as any form of corresponding RNA. For example, a nucleic acid encoding a TCRβ protein can comprise a recombinant nucleic acid encoding such a protein. The nucleic acid can be a non-naturally occurring nucleic acid created artificially (such as by assembling, cutting, ligating or amplifying sequences). It can be double-stranded or single-stranded.

The invention further provides for nucleic acids that are complementary to a TCRβ molecule. Complementary nucleic acids can hybridize to the nucleic acid sequence described above under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

TCRβ nucleic acid sequences can vary due to recombination of V, D and J gene segments during T-cell maturation. Sequence variations arising from somatic hypermutation can give rise to further sequence complexity. Correspondingly, nucleic acid molecules encoding T cell receptor protein sequences are genomic or mRNA-sequences that encode antigen-specificity-conferring TCR peptide sequences. In one embodiment, the invention is directed to a T cell receptor beta nucleic acid. In another embodiment, the invention is directed to a T cell receptor alpha nucleic acid, a T cell receptor gamma nucleic acid, or a T cell receptor delta nucleic acid.

Complementarity determining regions (CDR), or hypervariable regions, are sequences in the variable domains of antigen receptors (e.g., T cell receptor and immunoglobulin) that can complement an antigen. The chain of each antigen receptor contains three CDRs (CDR1, CDR2, and CDR3). In one embodiment, the T-cell receptor beta gene sequence of a T-cell is determined. In another embodiment, the sequence of the CDR3 region of the T-cell receptor beta of a T-cell is determined. In a further embodiment, the sequence of the CDR2 region of the T-cell receptor beta of a T-cell is determined. In another embodiment, the sequence of the CDR1 region of the T-cell receptor beta of a T-cell is determined.

High Throughput Sequencing

One aspect of the present invention provides a method for determining the sequence of T-cell receptor beta gene sequences. One aspect of the present invention provides a method for determining donor reactive T-cell receptor beta gene sequences in the subject. In another embodiment, the invention provides a method for determining recipient reactive T-cell receptor beta gene sequences in the subject.

In one embodiment, the T-cell receptor beta gene sequences are determined by high-throughout sequencing. In another embodiment, the T-cell receptor beta gene sequences are determined by high-throughout sequencing using the ImmunoSEQ technique from Adaptive™. High-throughout sequencing can include, but is not limited to, one or more rounds of nucleic acid amplification, spatially isolating individual nucleic acids, and sequencing nucleic acids. The nucleic acids can be DNA or RNA.

Methods for isolation of nucleic acids from a pool are known to those skilled in the art and include, but are not limited to subcloning nucleic acid into DNA vectors and transforming bacteria (bacterial cloning), spatial separation of the molecules in two dimensions on a solid substrate, spatial separation of the molecules in three dimensions in a solution within micelles (e.g. oil emulsions with or without immobilizing the molecules on a solid surface such as beads), or using microreaction chambers in, for example, microfluidic or nano-fluidic chips.

Methods for amplification of nucleic acids are known to one of skill in the art and include, but is not limited to, PCR amplification, bacterial growth of isolated colonies transformed with nucleic acid, amplification on a slide, and amplification on a bead.

Any technique for sequencing nucleic acid known to those skilled in the art can be used in the methods of the provided invention. High-throughput or “deep” sequencing methods can provide hundreds of millions bases of sequence information. High throughput sequencing uses massively parallelled sequencing reactions, reading out large numbers of relatively short sequences which are aligned by computing. Commercially available platforms are available for high-throughput sequencing and are known to one of skill in the art.

Sequences can be determined that originate from a single molecule or that originate from amplifications from a single molecule. Millions of independent amplifications of single molecules can be performed in parallel either on a solid surface or in tiny compartments in water/oil emulsion. It is possible to generate many millions of reads in one run. Various methods known in the art can be used during amplification to ensure that the frequency of the DNA sequences measured matches the frequency of the DNA sequence in the original sample. An algorithm can be used to combine reads together to more accurately determine the frequency of a DNA sequence in the starting material.

DNA sequencing techniques include, but are not limited to classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, SOLiD sequencing, true single molecule sequencing (tSMS), SOLEXA sequencing, single molecule real time (SMRT™) sequencing, nanopore sequencing, chemical-sensitive field effect transistor (chemFET) array sequencing, and ImmunoSEQ (Adaptive™).

In one embodiment, the T-cell receptor beta gene sequences are determined by high-throughout sequencing using the ImmunoSEQ technique from Adaptive™. CDR3 regions of the T-cell receptor beta gene can be amplified with solid-phase PCR primers specific for the variable and joining regions of the T-cell receptor beta gene, followed by high throughput sequencing. These sequences can provide the repertoire of T-cell receptor beta sequences present in the sample. In some embodiments, the full sequence of the variable regions can be sequenced to identify and quantify a T-cell receptor sequence.

In one embodiment, the invention provides a method for determining the frequency of one or more T-cells carrying a particular T-cell receptor beta gene. In another embodiment the T-cells are isolated from blood. In a more preferred embodiment, the lymphocytes are peripheral blood mononuclear cells (PBMC), especially T lymphocytes, or lymphocytes obtained by tissue biopsy. In a further embodiment, the isolated lymphocytes are regulatory T cells. In another embodiment, the isolated lymphocytes are effector T cells. In another embodiment, isolated lymphocytes are a combination of both effector and regulatory T cells.

In one embodiments, nucleic acids are analyzed from a population or subset of cells. In one embodiment, a method to separate cells, for example by using a cell surface marker, can be used. For example, cells can be isolated by cell sorting flow-cytometry, flow-sorting, fluorescent activated cell sorting (FACS), bead based separation such as magnetic cell sorting (MACS; e.g., using antibody coated magnetic particles), size-based separation (e.g., a sieve, an array of obstacles, or a filter), sorting in a microfluidics device, antibody-based separation, sedimentation, affinity adsorption, affinity extraction, or density gradient centrifugation. Cells can be purified by laser capture microdissection. Sorting can be based on cell size, morphology, or intracellular or extracellular markers. Methods for isolating or sorting T-cells are described in U.S. Pat. No. 4,777,145, U.S. Pat. No. 8,004,661, U.S. Pat. No. 5,367,474, U.S. Pat. No. 4,347,935, each of which is herein incorporated by reference in their entireties.

In one embodiment, the population or subset of cells can be a population or subset of T-cells. In one embodiment, the method can comprise detecting the presence of a marker gene (such as, CD4, or CD8) polypeptide expression. Polypeptide expression includes the presence of a marker gene polypeptide sequence, or the presence of an elevated quantity of marker gene polypeptide. In one embodiment, the subset or population of T-cells can be CD4 positive cells. In a further embodiment, the subset or population of T-cells can be CD8 positive cells.

The present invention provides methods for separating, enriching, isolating or purifying T-cells from a tissue or mixed population of cells. The methods comprise obtaining a mixed population of cells, contacting the population of cells with an agent that binds to CD4 or CD8, and separating the subpopulation of cells that are bound by the agent from the subpopulation of cells that are not bound by the agent, wherein the subpopulation of cells that are bound by the agent is enriched for CD4-positive T-cells or CD8-positive T-cells.

The methods also comprise obtaining a mixed population of cells, contacting the population of cells with a dye that labels the cells, and separating the subpopulation of cells with a lower amount of dye from the subpopulation of cells with a higher amount of dye, wherein the subpopulation of cells that have a lower amount of dye is enriched for cells that have proliferated after the labeling of the cells with the dye.

The methods for separating, enriching, isolating or purifying T-cells from a mixed population of cells according to the invention may be combined with other methods for separating, enriching, isolating or purifying T-cells or immune cells that are known in the art. For example, the methods described herein may be performed in conjunction with techniques that use other T-cell markers. For example, an additional selection step may be performed either before, after, or simultaneously with the CD-4 or CD-8 selection step, in which a second agent, such as an antibody, that binds to a second T-cell marker, or a dye that labels the cells, is used. The second cell marker or dye may be any cell marker or dye known in the art. In one embodiment, the second T-cell marker is CD-3. In another embodiment, the dye is Horizon Violet Proliferation Dye 450. In another embodiment, the dye is carboxyfluorescein succinimidyl ester (CFSE). The mixed population of cells can be any source of cells from which to obtain CD-4 or CD-8-positive T-cells, including, but not limited to a blood or urine from a subject.

The agent used can be any agent that binds to CD-4 or CD-8, as described above. The term “Agent” includes, but is not limited to, small molecule drugs, peptides, proteins, peptidomimetic molecules, and antibodies. It also includes any CD-4, or CD-8 binding molecule that is labeled with a detectable moiety, such as a histological stain, an enzyme substrate, a fluorescent moiety, a magnetic moiety or a radio-labeled moiety. Such “labeled” agents are particularly useful for embodiments involving isolation or purification of CD-4 or CD-8 positive cells, or detection of CD-4 or CD-8 positive cells. In some embodiments, the agent is an antibody that binds to CD-4 or CD-8.

There are many cell separation techniques known in the art, and any such technique may be used. For example magnetic cell separation techniques can be used if the agent is labeled with an iron-containing moiety. Cells may also be passed over a solid support that has been conjugated to an agent that binds to CD-4 or CD-8, such that the CD-4 or CD-8 positive cells will be selectively retained on the solid support. Cells may also be separated by density gradient methods, particularly if the agent selected significantly increases the density of the CD-4 or CD-8 positive cells to which it binds. For example, the agent can be a fluorescently labeled antibody against CD-4 or CD-8, and the CD-4 or CD-8 positive T-cells cells are separated from the other cells using fluorescence activated cell sorting (FACs).

In one embodiment, T-cell receptor beta gene sequences are determined from donor reactive T-cells. In one embodiment, donor reactive T-cells can be identified by a mixed lymphocyte reaction (MLR). In a MLR, PBMCs from the organ donor (the stimulator) and a first T-cell population from the recipient (the responder) are mixed in a culture dish and incubated. PBMCs from the organ donor can be treated with irradiation, for example, to render them non-proliferative. PBMCs from the organ donor and the T-cell population from the recipient can be labeled with different dyes. For example, PBMCs from the organ donor can be labeled with CFSE and the T-cell population from the recipient can be labeled with Horizon Violet Proliferation Dye 450, or vice versa. The labeled PBMCs from the donor and T-cells from the recipient can be separated by cell separation techniques, such as Flourescence Activated Cell Sorting (FACS). A second T-cell population can be isolated from the first T-cell population of the recipient. In one embodiment, the second T-cell population comprises cells from the first T-cell population that proliferate in response to the PBMCs from the donor. The second T-cell population can be isolated due to the dilution of the labeling dye in these cells following proliferation. In some embodiments, the MLR can be performed with lymphocytes. In one embodiment, the second T-cell population can be sorted into a subset or population of T-cells that are be CD4 positive cells. In a further embodiment, the second T-cell population can be sorted into a subset or population of T-cells that are be CD8 positive cells. In a further embodiment, the second T-cell population can be sorted into a subset or population of T-cells that are be CD3 positive cells. In one embodiment, T-cell receptor beta gene sequences are determined from the first T-cell population (unstimulated) from the recipient. In another embodiment, T-cell receptor beta gene sequences are determined from the second T-cell population from the recipient (stimulated).

In one embodiment, donor reactive T-cells are identified by comparing the first T-cell population with the second T-cell population, wherein the donor reactive T-cells comprise T-cells with a frequency of about 0.01% in the second T-cell population and about 5-fold higher frequency in the second T-cell population compared to the first T-cell population. In another embodiment donor reactive T-cells comprise T-cells with a frequency of at least about 0.001%, of at least about 0.002%, of at least about 0.003%, of at least about 0.004%, of at least about 0.005%, of at least about 0.006%, of at least about 0.007%, of at least about 0.008%, of at least about 0.009%, of at least about 0.01%, of at least about 0.02%, of at least about 0.03%, of at least about 0.04%, of at least about 0.05%, of at least about 0.06%, of at least about 0.07%, of at least about 0.08%, of at least about 0.09%, of at least about 0.1%, in the second T-cell population.

In one embodiment donor reactive T-cells comprise T-cells with a frequency of at least 0.001%, of at least 0.002%, of at least 0.003%, of at least 0.004%, of at least 0.005%, of at least 0.006%, of at least 0.007%, of at least 0.008%, of at least 0.009%, of at least 0.01%, of at least 0.02%, of at least 0.03%, of at least 0.04%, of at least 0.05%, of at least 0.06%, of at least 0.07%, of at least 0.08%, of at least 0.09%, of at least 0.1%, in the second T-cell population.

In one embodiment, donor reactive T-cells comprise T-cells with about 1.5-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold, higher frequency in the second T-cell population compared to the first T-cell population.

In one embodiment, donor reactive T-cells comprise T-cells with at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold, higher frequency in the second T-cell population compared to the first T-cell population.

In one embodiment, T-cell receptor beta gene sequences are determined from recipient reactive T-cells. Recipient reactive T-cells can be identified by a mixed lymphocyte reaction (MLR). In a MLR, PBMCs from the recipient (the stimulator) and a first T-cell population from the organ donor (the responder) are mixed in a culture dish and incubated. PBMCs from the recipient can be treated with irradiation, for example, to render them non-proliferative. PBMCs from the recipient and the T-cell population from the organ donor can be labeled with different dyes. For example, PBMCs from the recipient can be labeled with CFSE and the T-cell population from the organ donor can be labeled with Horizon Violet Proliferation Dye 450, or vice versa. The labeled PBMCs from the recipient and T-cells from the organ donor can be separated by cell separation techniques, such as Flourescence Activated Cell Sorting (FACS). A second T-cell population can be isolated from the first T-cell population of the organ donor. In one embodiment, the second T-cell population comprises cells from the first T-cell population that proliferate in response to the PBMCs from the recipient. The second T-cell population can be isolated due to the dilution of the labeling dye in these cells following proliferation. In some embodiments, the MLR can be performed with lymphocytes. In one embodiment, the second T-cell population can be sorted into a subset or population of T-cells that are be CD4 positive cells. In a further embodiment, the second T-cell population can be sorted into a subset or population of T-cells that are be CD8 positive cells. In one embodiment, T-cell receptor beta gene sequences are determined from the first T-cell population from the recipient (unstimulated). In another embodiment, T-cell receptor beta gene sequences are determined from the second T-cell population from the recipient (stimulated).

In one embodiment, recipient reactive T-cells are identified by comparing the first T-cell population with the second T-cell population, wherein the recipient reactive T-cells comprise T-cells with a frequency of about 0.01% in the second T-cell population and about 5-fold higher frequency in the second T-cell population compared to the first T-cell population. In another embodiment donor reactive T-cells comprise T-cells with a frequency of at least about 0.001%, of at least about 0.002%, of at least about 0.003%, of at least about 0.004%, of at least about 0.005%, of at least about 0.006%, of at least about 0.007%, of at least about 0.008%, of at least about 0.009%, of at least about 0.01%, of at least about 0.02%, of at least about 0.03%, of at least about 0.04%, of at least about 0.05%, of at least about 0.06%, of at least about 0.07%, of at least about 0.08%, of at least about 0.09%, of at least about 0.1%, in the second T-cell population.

In one embodiment recipient reactive T-cells comprise T-cells with a frequency of at least 0.001%, of at least 0.002%, of at least 0.003%, of at least 0.004%, of at least 0.005%, of at least 0.006%, of at least 0.007%, of at least 0.008%, of at least 0.009%, of at least 0.01%, of at least 0.02%, of at least 0.03%, of at least 0.04%, of at least 0.05%, of at least 0.06%, of at least 0.07%, of at least 0.08%, of at least 0.09%, of at least 0.1%, in the second T-cell population.

In one embodiment, recipient reactive T-cells comprise T-cells with about 1.5-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold, higher frequency in the second T-cell population compared to the first T-cell population.

In one embodiment, recipient reactive T-cells comprise T-cells with at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold, higher frequency in the second T-cell population compared to the first T-cell population.

T-cell receptor beta gene sequences may be in the form of nucleic acid sequences or amino acid sequences. The sequencing reaction will render nucleic acid sequences, which can then be translated to amino acid sequences using the known triplet code. Comparison is can be performed on the level of amino acid sequences or on the level of nucleic acid sequences. Each T-cell receptor beta gene sequences can be aligned base by base or amino acid by amino acid. Methods of alignment are known in the art; the FASTA and BLAST programme packages available publicly are examples.

Methods of Using T Cell Receptor Beta Sequences

The identification or determination of donor or recipient specific T-cell receptor sequences allows cells carrying these sequences to be tracked or monitored in the recipient of an organ transplant. In one embodiment, the clonal composition of donor or recipient specific T cells in the clinical course of patients receiving an organ transplant is traced. The method described herein provides qualitative and quantitative accuracy regarding the T cell receptor repertoire of the immune response within a specific individual.

In one embodiment, non-tolerance of an organ transplant in a recipient is determined. In another embodiment, rejection of an organ transplant in a recipient is determined. In another embodiment, rejection of an organ transplant in a recipient is diagnosed. In another embodiment, rejection of an organ transplant in a recipient is predicted. In one embodiment, an immunosuppressive therapy is administered to a subject when the frequency of donor reactive T-cells in the post transplant sample is equal to or higher than the frequency of donor reactive T-cells in a pre-transplant sample.

In one embodiment, the presence of donor reactive T-cells in a post transplant sample indicates non-tolerance or rejection of an organ transplant. In one embodiment, the post-transplant sample is urine. In another embodiment, the post-transplant sample is a tissue biopsy of the transplanted organ.

In one embodiment, the post-transplant sample is unstimulated. In another embodiment, the pre-transplant sample is unstimulated. In one embodiment, a statistical test can be used to determine if the frequency of donor reactive T-cells in the post transplant sample is equal to or higher than the frequency of donor reactive T-cells in a pre-transplant sample. For example, the frequency of donor reactive T-cells in the post-transplant and pre-transplant samples can be compared using a two-sided Fisher's exact test. In further embodiments, other statistical tests can be used to determine if the frequency of donor reactive T-cells in the post transplant sample is equal to or higher than the frequency of donor reactive T-cells in a pre-transplant sample. In one embodiment, the odds ratio of an increase or decrease in donor-reactive T-cells in the post transplant sample as compared a to pre-transplant sample is calculated.

In one embodiment, an immunosuppressive therapy is administered when the odds ratio of donor-reactive T-cells relative to pre-transplant is at least 1. In another embodiment, an immunosuppressive therapy is administered when the odds ratio of donor-reactive T-cells relative to pre-transplant is at least 1.5, at least 2, at least 2.5, at least 3, at least 4, at least 5, at least 6, at least 7, at least 8, at least 9, or at least 10. In another embodiment, an immunosuppressive therapy is administered when the odds ratio of donor-reactive T-cells relative to pre-transplant is about 1.5, about 2, about 2.5, about 3, about 4, about 5, about 6, about 7, about 8, about 9, or about 10.

In one embodiment, non-tolerance of an organ transplant in a recipient is determined when the odds ratio of donor-reactive T-cells relative to pre-transplant is at least 1. In another embodiment, non-tolerance of an organ transplant in a recipient is determined when the odds ratio of donor-reactive T-cells relative to pre-transplant is at least 1.5, at least 2, at least 2.5, at least 3, at least 4, at least 5, at least 6, at least 7, at least 8, at least 9, or at least 10. In another embodiment, non-tolerance of an organ transplant in a recipient is determined when the odds ratio of donor-reactive T-cells relative to pre-transplant is about 1.5, about 2, about 2.5, about 3, about 4, about 5, about 6, about 7, about 8, about 9, or about 10.

In one embodiment, rejection of an organ transplant in a recipient is determined in a recipient is determined when the odds ratio of donor-reactive T-cells relative to pre-transplant is at least 1. In another embodiment, rejection of an organ transplant in a recipient is determined when the odds ratio of donor-reactive T-cells relative to pre-transplant is at least 1.5, at least 2, at least 2.5, at least 3, at least 4, at least 5, at least 6, at least 7, at least 8, at least 9, or at least 10. In another embodiment, rejection of an organ transplant in a recipient is determined when the odds ratio of donor-reactive T-cells relative to pre-transplant is about 1.5, about 2, about 2.5, about 3, about 4, about 5, about 6, about 7, about 8, about 9, or about 10.

In one embodiment, rejection of an organ transplant in a recipient is diagnosed when the odds ratio of donor-reactive T-cells relative to pre-transplant is at least 1. In another embodiment, rejection of an organ transplant in a recipient is diagnosed when the odds ratio of donor-reactive T-cells relative to pre-transplant is at least 1.5, at least 2, at least 2.5, at least 3, at least 4, at least 5, at least 6, at least 7, at least 8, at least 9, or at least 10. In another embodiment, rejection of an organ transplant in a recipient is diagnosed when the odds ratio of donor-reactive T-cells relative to pre-transplant is about 1.5, about 2, about 2.5, about 3, about 4, about 5, about 6, about 7, about 8, about 9, or about 10.

In one embodiment, rejection of an organ transplant in a recipient is predicted when the odds ratio of donor-reactive T-cells relative to pre-transplant is at least 1. In another embodiment, rejection of an organ transplant in a recipient is predicted when the odds ratio of donor-reactive T-cells relative to pre-transplant is at least 1.5, at least 2, at least 2.5, at least 3, at least 4, at least 5, at least 6, at least 7, at least 8, at least 9, or at least 10. In another embodiment, rejection of an organ transplant in a recipient is predicted when the odds ratio of donor-reactive T-cells relative to pre-transplant is about 1.5, about 2, about 2.5, about 3, about 4, about 5, about 6, about 7, about 8, about 9, or about 10.

In one embodiment, organ transplant rejection is diagnosed by the presence of donor reactive T-cells in a post transplant sample. In one embodiment, the post-transplant sample is urine. In another embodiment, the post-transplant sample is a tissue biopsy of the transplanted organ. The detection of donor reactive T-cells in a post transplant sample can be performed in a variety of methods, including, but not limited to high throughput sequencing of the post-transplant sample. In one embodiment, organ transplant rejection is diagnosed by tracking the presence of donor reactive T-cells in a post transplant samples over time. In one embodiment, organ transplant rejection can be diagnosed when the presence of donor reactive T-cells in a post transplant samples increases over time.

In one embodiment, non-tolerance of an organ transplant in a recipient is determined by the presence of donor reactive T-cells in a post transplant sample. In another embodiment, rejection of an organ transplant in a recipient is determined by the presence of donor reactive T-cells in a post transplant sample. In another embodiment, rejection of an organ transplant in a recipient is diagnosed by the presence of donor reactive T-cells in a post transplant sample. In another embodiment, rejection of an organ transplant in a recipient is predicted by the presence of donor reactive T-cells in a post transplant sample. In one embodiment, an immunosuppressive therapy is administered to a subject when the presence of donor reactive T-cells is detected in a post transplant sample. In one embodiment, the post-transplant sample is urine. In another embodiment, the post-transplant sample is a tissue biopsy of the transplanted organ. The detection of donor reactive T-cells in a post transplant sample can be performed in a variety of methods, including, but not limited to high throughput sequencing of the post-transplant sample. In one embodiment, non-tolerance of an organ transplant in a recipient is determined by tracking the presence of donor reactive T-cells in a post transplant samples over time. In one embodiment, non-tolerance of an organ transplant in a recipient is determined when the presence of donor reactive T-cells in a post transplant samples increases over time. In one embodiment, rejection of an organ transplant in a recipient is determined by tracking the presence of donor reactive T-cells in a post transplant samples over time. In one embodiment, rejection of an organ transplant in a recipient is determined when the presence of donor reactive T-cells in a post transplant samples increases over time. In one embodiment, rejection of an organ transplant in a recipient is diagnosed by tracking the presence of donor reactive T-cells in a post transplant samples over time. In one embodiment, rejection of an organ transplant in a recipient is diagnosed when the presence of donor reactive T-cells in a post transplant samples increases over time. In one embodiment, rejection of an organ transplant in a recipient is predicted by tracking the presence of donor reactive T-cells in a post transplant samples over time. In one embodiment, rejection of an organ transplant in a recipient is predicted when the presence of donor reactive T-cells in a post transplant samples increases over time.

In one embodiment, organ transplant tolerance is predicted. In one embodiment, organ transplant tolerance is identified. In one embodiment, administration of an immunosuppressive therapy is withdrawn or reduced when the frequency of donor reactive T-cells in the post transplant sample is equal to or less than the frequency of donor reactive T-cells in a pre-transplant sample. In one embodiment, the post-transplant sample is unstimulated. In another embodiment, the pre-transplant sample is unstimulated. In one embodiment, a statistical test can be used to determine if the frequency of donor reactive T-cells in the post transplant sample is equal to or lower than the frequency of donor reactive T-cells in a pre-transplant sample. For example, the frequency of donor reactive T-cells in the post-transplant and pre-transplant samples can be compared using a two-sided Fisher's exact test. In further embodiments, other statistical tests can be used to determine if the frequency of donor reactive T-cells in the post transplant sample is equal to or lower than the frequency of donor reactive T-cells in a pre-transplant sample. In one embodiment, the odds ratio of an increase or decrease in donor-reactive T-cells in the post transplant sample as compared to pre-transplant is calculated.

In one embodiment, administration of an immunosuppressive therapy is withdrawn or reduced when the odds ratio of donor-reactive T-cells relative to pre-transplant is less than 1. In another embodiment, administration of an immunosuppressive therapy is withdrawn or reduced when the odds ratio of donor-reactive T-cells relative to pre-transplant is less than 1, less than 0.5, less than 0.4, less than 0.3, less than 0.2, or less than 0.1. In another embodiment, administration of an immunosuppressive therapy is withdrawn or reduced when the odds ratio of donor-reactive T-cells relative to pre-transplant is about 0.5, about 0.4, about 0.3, about 0.2, or about 0.1.

In one embodiment, organ transplant tolerance is predicted when the odds ratio of donor-reactive T-cells relative to pre-transplant is less than 1. In another embodiment, organ transplant tolerance is predicted when the odds ratio of donor-reactive T-cells relative to pre-transplant is less than 1, less than 0.5, less than 0.4, less than 0.3, less than 0.2, or less than 0.1. In another embodiment, organ transplant tolerance is predicted when the odds ratio of donor-reactive T-cells relative to pre-transplant is about 0.5, about 0.4, about 0.3, about 0.2, or about 0.1.

In one embodiment, organ transplant tolerance is identified when the odds ratio of donor-reactive T-cells relative to pre-transplant is less than 1. In another embodiment, organ transplant tolerance is identified when the odds ratio of donor-reactive T-cells relative to pre-transplant is less than 1, less than 0.5, less than 0.4, less than 0.3, less than 0.2, or less than 0.1. In another embodiment, organ transplant tolerance is identified when the odds ratio of donor-reactive T-cells relative to pre-transplant is about 0.5, about 0.4, about 0.3, about 0.2, or about 0.1.

In one embodiment, the frequency of donor reactive T-cells can be compared between two post-transplant samples. In another embodiment, the post-transplant sample is unstimulated. In another embodiment, the post-transplant sample is blood. In a further embodiment, the post-transplant sample is urine. In one embodiment, a statistical test can be used to determine if the frequency of donor reactive T-cells in a second post transplant sample is compared to the frequency of donor reactive T-cells in a first post transplant sample. For example, the frequency of donor reactive T-cells in the a second post transplant and first post transplant samples can be compared using a two-sided Fisher's exact test. In further embodiments, other statistical tests can be used to determine if the frequency of donor reactive T-cells in the second post transplant sample is lower than, equal to or higher than the frequency of donor reactive T-cells in a first post transplant sample. In one embodiment, the odds ratio of an increase or decrease in donor-reactive T-cells in the second post transplant sample as compared to the first post transplant is calculated. Post-transplant samples can be obtained from a subject 1 week, 2 weeks, 3 weeks, 4 weeks, 5 weeks, 6 weeks, 2 months, 3 months, 4 months, 5 months, 6 months, 7 months, 8 months, 9 months, 10 months, 11 months, 12 months, 18 months, 24 months, 3 years, 4 years, 5 years, 10 years, 20 years, 30 years, 40 years, or 50 years after the subject receives an organ transplant from a donor.

In one embodiment, graft versus host disease is treated. In one embodiment, graft versus host disease is predicted. In one embodiment, graft versus host disease is diagnosed. In some embodiments, graft versus host disease is caused by recipient reactive T-cells from the donor organ. In one embodiment, the donor organ is a hematopoietic cell transplant. In another embodiment the donor organ is a heart, a kidney, a liver, a lung or lungs, a pancreas, an intestine, a stomach, a testis, a thymus. In some embodiments, recepeint reactive T-cells can migrate out of the donor organ. In one embodiment, an immunosuppressive therapy is administered to a subject when recipent reactive donor T-cells are detected in a post transplant sample. In one embodiment, the post-transplant sample is unstimulated.

In one embodiment, absence of graft versus host disease is predicted. In one embodiment, administration of an immunosuppressive therapy is withdrawn or reduced when when recipent reactive donor T-cells are not detected in a post transplant sample. In one embodiment, the post-transplant sample is unstimulated.

In one embodiment, recipient tolerance is predicted. In one embodiment, administration of an immunosuppressive therapy is withdrawn or reduced when when recipent reactive donor T-cells are not detected in a post transplant sample. In one embodiment, the post-transplant sample is unstimulated. In another embodiment, the post-transplant sample is blood. In a further embodiment, the post-transplant sample is urine. In another embodiment, the post-transplant sample is a tissue biopsy. In another embodiment, the post-transplant sample is a tissue biopsy of the transplanted organ.

In one embodiment, recipient non-tolerance is predicted. In one embodiment, administration of an immunosuppressive therapy is administered when when recipent reactive donor T-cells are detected in a post transplant sample. In one embodiment, the post-transplant sample is unstimulated. In another embodiment, the post-transplant sample is blood. In a further embodiment, the post-transplant sample is urine. In another embodiment, the post-transplant sample is a tissue biopsy. In another embodiment, the post-transplant sample is a tissue biopsy of the transplanted organ.

In one embodiment, the frequency of recipient reactive T-cells can be compared between two post-transplant samples. In another embodiment, the post-transplant sample is unstimulated. In another embodiment, the post-transplant sample is blood. In a further embodiment, the post-transplant sample is urine. In another embodiment, the post-transplant sample is a tissue biopsy. In another embodiment, the post-transplant sample is a tissue biopsy of the transplanted organ. In one embodiment, a statistical test can be used to compare the frequency of recipient reactive T-cells in a second post transplant sample to the frequency of recipient reactive T-cells in a first post transplant sample. For example, the frequency of recipient reactive T-cells in the a second post transplant and first post transplant samples can be compared using a two-sided Fisher's exact test. In further embodiments, other statistical tests can be used to determine if the frequency of recipient reactive T-cells in the second post transplant sample is lower than, equal to or higher than the frequency of recipient reactive T-cells in a first post transplant sample. In one embodiment, the odds ratio of an increase or decrease in recipient reactive T-cells in the second post transplant sample as compared to the first post transplant is calculated. Post-transplant samples can be obtained from a subject 1 week, 2 weeks, 3 weeks, 4 weeks, 5 weeks, 6 weeks, 2 months, 3 months, 4 months, 5 months, 6 months, 7 months, 8 months, 9 months, 10 months, 11 months, 12 months, 18 months, 24 months, 3 years, 4 years, 5 years, 10 years, 20 years, 30 years, 40 years, or 50 years after the subject receives an organ transplant from a donor.

An aspect of the invention provides for a method of treating organ transplant rejection in a subject in need thereof comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) administering an immunosuppressive therapy to the subject when the frequency of donor reactive T-cells in the post-transplant sample is equal to or higher than the frequency of T-cells determined in (b).

An aspect of the invention provides for a method of treating organ transplant rejection in a subject in need thereof comprising: (a) determining donor reactive T-cell receptor beta gene sequences in the subject; (b) determining the frequency of the donor reactive T-cell receptor beta gene sequences of (a) in a pre-transplant sample of the subject; (c) determining the frequency of donor reactive T-cell receptor beta gene sequences in a post-transplant sample from the subject; and (d) administering an immunosuppressive therapy to the subject when the frequency of donor reactive T-cell receptor beta gene sequences in the post-transplant sample is equal to or higher than the frequency of donor reactive T-cell receptor beta gene sequences in the pre-transplant sample.

An aspect of the invention provides for a method of diagnosing organ transplant rejection in a subject comprising: (a) determining donor reactive T-cell receptor beta gene sequences in the subject; (b) determining the frequency of donor reactive T-cells in a post-transplant sample of the subject carrying the gene sequences in (a); and (c) diagnosing organ transplant rejection in a subject when donor reactive T-cells are detected in the post-transplant sample.

An aspect of the invention provides for a method of reducing transplant rejection in a subject, wherein the subject has undergone an organ transplant, comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) administering an immunosuppressive therapy to the subject when the frequency of donor reactive T-cells in the post-transplant sample is equal to or higher than the frequency of T-cells determined in (b).

An aspect of the invention provides for a method of determining non-tolerance to an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) determining non-tolerance of the organ transplant when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of determining rejection of an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) determining rejection of the organ transplant when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of diagnosing organ transplant rejection in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) diagnosing organ transplant rejection in a subject when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of predicting organ transplant rejection in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) predicting the presence of organ transplant rejection in a subject when the frequency of donor reactive T-cells in the post-transplant sample is equal to or higher than the frequency of T-cells determined in (b). In one embodiment, the method further comprising administering an immunosuppressive therapy to the subject.

An aspect of the invention provides for a method of predicting organ transplant tolerance in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) predicting tolerance to an organ transplant in a subject when the frequency of donor reactive T-cells in the post-transplant sample is equal to or lower than the frequency of T-cells determined in (b). In one embodiment, the method further comprises reducing or withdrawing the administration of an immunosuppressive therapy to the subject.

An aspect of the invention provides for a method of identifying tolerance to an organ transplant in a subject comprising: (a) determining donor reactive T-cell receptor beta gene sequences in the subject; (b) determining the frequency of T-cells in a pre-transplant sample of the subject carrying the gene sequences in (a); (c) determining the frequency of donor reactive T-cells in a post-transplant sample from the subject; and (d) identifying tolerance to an organ transplant when the frequency of donor reactive T-cells in the post-transplant sample is equal to or lower than the frequency of T-cells determined in (b). In one embodiment, the method further comprises reducing or withdrawing the administration of an immunosuppressive therapy to the subject.

An aspect of the invention provides for a method of predicting organ transplant tolerance in a subject comprising: (a) determining donor reactive T-cell receptor beta gene sequences in the subject; (b) determining the frequency of T-cells carrying the gene sequences in (a) in a first post-transplant sample from the subject; (c) determining the frequency of T-cells carrying the gene sequences in (a) in a second post-transplant sample from the subject; and (d) predicting tolerance to an organ transplant in a subject when the frequency of T-cells determined in (c) is equal to or lower than the frequency of T-cells determined in (b). In one embodiment, the method further comprises reducing or withdrawing the administration of an immunosuppressive therapy to the subject.

An aspect of the invention provides for a method of treating graft versus host disease in an organ transplant recipient in need thereof comprising: (a) determining recipient reactive T-cell receptor beta gene sequences in a pre-transplant sample of an organ donor; (b) determining the frequency of donor T-cells in a post-transplant sample of the organ transplant recipient carrying the gene sequences in (a); and (c) administering an immunosuppressive therapy to the organ transplant recipient when recipient reactive donor T-cells are detected in the post-transplant sample.

An aspect of the invention provides for a method of predicting recipient tolerance in a subject comprising: (a) determining recipient reactive T-cell receptor beta gene sequences in a pre-transplant sample of an organ donor; (b) determining the frequency of donor T-cells in a post-transplant sample of the subject carrying the gene sequences in (a); and (c) predicting recipient tolerance in a subject when recipient reactive donor T-cells are not detected in the post-transplant sample.

An aspect of the invention provides for a method of predicting recipient non-tolerance in a subject comprising: (a) determining recipient reactive T-cell receptor beta gene sequences in a pre-transplant sample of an organ donor; (b) determining the frequency of donor T-cells in a post-transplant sample of the subject carrying the gene sequences in (a); and (c) predicting recipient non-tolerance in a subject when recipient reactive donor T-cells are detected in the post-transplant sample.

In one embodiment, the method further comprises determining the frequency of donor reactive T-cells in a subsequent post-transplant sample from the subject. In another embodiment, the method further comprises comparing the frequency of donor reactive T-cells in the post-transplant sample to the frequency of donor reactive T-cells in the subsequent post-transplant sample. In another embodiment, the pre-transplant sample is collected before the subject undergoes an organ transplant with an organ from the organ donor. In one embodiment, the post-transplant sample is collected after the subject undergoes an organ transplant with an organ from an organ donor. In another embodiment, the post-transplant sample is blood. In another embodiment, the pre-transplant sample is blood. In a further embodiment, the post-transplant sample is urine. In another embodiment, the post-transplant sample is a tissue sample of the transplanted organ. In another embodiment, the T cell population further comprises CD4 T-cells. In another embodiment, the T cell population further comprises CD8 T-cells. In another embodiment the subject is a human. In another embodiment, the T-cell receptor beta gene sequences comprise T-cell receptor beta CDR3 gene sequences.

In one embodiment, the immunosuppressive therapy is a glucocorticoid, a cytostatic agent, an antibody, an immunophilin modulator, an interferon, plasmapheresis, or a combination thereof. In another embodiment, the glucocorticoid is methylprednisolone, corticosteroid, prednisone, prednisolone, dexamethasone, or betametasone. In a further embodiment, the cytostatic is methotrexate, azathioprine, mercaptopurine, dactinomycin, anthracyclines, mitomycin C, bleomycin, mithramycin, mycophenolate mofetil. In another embodiment, the antibody is a chimeric antibody, a humanized antibody, or a fully human antibody. In another embodiment, the antibody is thymoglobulin, Atgam, Muromonab-CD3, basiliximab, daclizumab, rituximab, or intravenous immunoglobulin. In another embodiment, the immunophilin modulator is cyclosporine, sirolimus, tacrolimus. In a further embodiment, the interferon is interferon alpha 2a, interferon alpha 2b, interferon beta 1a, interferon beta 1b, interferon gamma 1b. In another embodiment, the immunosuppressive therapy is methylprednisolone, corticosteroid, thymoglobulin, basiliximab, rituximab, intravenous immunoglobulin, tacrolimus, mycophenolate, plasmapheresis, or a combination thereof.

In one embodiment the organ transplant is a heart transplant, a kidney transplant, a liver transplant, a lung transplant, a pancreas transplant, an intestine transplant, a stomach transplant, a testis transplant, a thymus transplant, a hematopoietic cell transplant, or combination thereof. In another embodiment, the organ transplant is a hematopoietic celltransplant, a tendon transplant, a cornea transplant, a skin transplant, a heart valve transplant, a nerve transplant, a vein transplant, a bone transplant, an Islets of Langerhans transplant, or a combination thereof. In another embodiment, the organ transplant is a face transplant, a hand transplant, a leg transplant, a penis transplant, a uterus transplant, an ovary transplant, a hematopoietic cell transplant, or a combination thereof. In another embodiment, the organ transplant is a combined kidney and hematopoietic cell transplant. In another embodiment, the organ transplant is an intestine transplant. In another embodiment, the organ transplant is a kidney transplant. In another embodiment, the organ transplant is a hematopoietic cell transplant.

Sequencing individual T-cell receptor beta gene sequences can distinguish different sequences and can have the sensitivity to detect quantitative changes in clonal expansion or deletion of T-cells carrying a particular T-cell receptor beta gene sequence. In one embodiment, the invention provides a method for determining the frequency of one or more T-cells carrying a particular T-cell receptor beta gene sequence in a sample of a subject. In another embodiment, the frequency of one or more T-cells carrying a particular T-cell receptor beta gene sequence in a sample of a subject can be determined at different time points. In one embodiment, the frequency of one or more T-cells carrying a particular T-cell receptor beta gene sequence can be determined before the subject receives an organ transplant from a donor (e.g., the frequency can be determined in a pre-transplant sample). In another embodiment, the frequency of one or more T-cells carrying a particular T-cell receptor beta gene sequence can be determined after the subject receives an organ transplant from a donor (for example, the frequency can be determined in a post-transplant sample). In another embodiment, the frequency of one or more T-cells carrying a particular T-cell receptor beta gene sequence can be determined 1 week, 2 weeks, 3 weeks, 4 weeks, 5 weeks, 6 weeks, 2 months, 3 months, 4 months, 5 months, 6 months, 7 months, 8 months, 9 months, 10 months, 11 months, 12 months, 18 months, 24 months, 3 years, 4 years, 5 years, 10 years, 20 years, 30 years, 40 years, or 50 years after the subject receives an organ transplant from a donor. In another embodiment the frequency of one or more T-cells carrying a particular T-cell receptor beta gene sequence can be tracked over time. For example, the frequency of one or more T-cells carrying a particular T-cell receptor beta gene sequence can be determined in a various post-transplant samples taken at various time points after the subject receives an organ transplant from a donor. In one embodiment, the T-cells are donor specific. In another embodiment, the T-cells are recipient specific.

In one embodiment, the invention provides a method for determining the frequency of a particular T-cell receptor beta gene sequence in a sample of a subject. In one embodiment, the frequency of a particular T-cell receptor beta gene sequence can be determined before the subject receives an organ transplant from a donor (e.g., the frequency can be determined in a pre-transplant sample). In another embodiment, the frequency of one or more T-cell receptor beta gene sequences can be determined after the subject receives an organ transplant from a donor (for example, the frequency can be determined in a post-transplant sample). In another embodiment, the frequency of one or more T-cell receptor beta gene sequences can be determined 1 week, 2 weeks, 3 weeks, 4 weeks, 5 weeks, 6 weeks, 2 months, 3 months, 4 months, 5 months, 6 months, 7 months, 8 months, 9 months, 10 months, 11 months, 12 months, 18 months, 24 months, 3 years, 4 years, 5 years, 10 years, 20 years, 30 years, 40 years, or 50 years after the subject receives an organ transplant from a donor. In another embodiment the frequency of one or more T-cell receptor beta gene sequences can be tracked over time. For example, the frequency of one or more T-cell receptor beta gene sequences can be determined in a various post-transplant samples taken at various time points after the subject receives an organ transplant from a donor. In one embodiment, the T-cell receptor beta gene sequences are donor specific. In another embodiment, the T-cell receptor beta gene sequences are recipient specific.

In one embodiment, the present invention provides a method for analysis of the T cell receptor repertoire of lymphocytes obtained from a subject. In another embodiment, the T cell receptor repertoire of lymphocytes obtained from a subject can be determined at different time points. In one embodiment, the T cell receptor repertoire of lymphocytes obtained from a subject can be determined before the subject receives an organ transplant from a donor (e.g., the frequency can be determined in a pre-transplant sample). In another embodiment, the T cell receptor repertoire of lymphocytes obtained from a subject can be determined after the subject receives an organ transplant from a donor (for example, the frequency can be determined in a post-transplant sample). In another embodiment, the T cell receptor repertoire of lymphocytes obtained from a subject can be determined 1 week, 2 weeks, 3 weeks, 4 weeks, 5 weeks, 6 weeks, 2 months, 3 months, 4 months, 5 months, 6 months, 7 months, 8 months, 9 months, 10 months, 11 months, 12 months, 18 months, 24 months, 3 years, 4 years, 5 years, 10 years, 20 years, 30 years, 40 years, or 50 years after the subject receives an organ transplant from a donor. In another embodiment the T cell receptor repertoire of lymphocytes obtained from a subject can be tracked over time. For example, the T cell receptor repertoire of lymphocytes obtained from a subject can be determined in a various post-transplant samples taken at various time points after the subject receives an organ transplant from a donor. In one embodiment, the T cell receptor repertoire is donor specific. In another embodiment, the T cell receptor repertoire is recipient specific.

Methods of Treatment

Pharmaceutical Compositions and Methods of Administration

In some embodiments, an immunosuppressive therapy can be supplied in the form of a pharmaceutical composition, comprising an isotonic excipient prepared under sufficiently sterile conditions for human administration. Choice of the excipient and any accompanying elements of the composition comprising an immunosuppressive therapy will be adapted in accordance with the route and device used for administration. In some embodiments, a composition comprising an immunosuppressive therapy can also comprise, or be accompanied with, one or more other ingredients that facilitate the delivery or functional mobilization of the immunosuppressive therapy.

These methods described herein are by no means all-inclusive, and further methods to suit the specific application is understood by the ordinary skilled artisan. Moreover, the effective amount of the compositions can be further approximated through analogy to compounds known to exert the desired effect.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A an immunosuppressive therapy can be administered to the subject one time (e.g., as a single injection or deposition). Alternatively, an immunosuppressive therapy can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12, 13, 14, 15, 16, 17, 18, 19, or 20 years, or a combination thereof. Furthermore, an immunosuppressive therapy can be co-administrated with another therapeutic.

An immunosuppressive therapy may also be used in combination with surgical or other interventional treatment regimens used for the treatment of transplant rejection.

The compositions of this invention can be formulated and administered to reduce the symptoms associated with an organ transplant rejection by any means that produce contact of the active ingredient with the agent's site of action in the body of a human or nonhuman subject. For example, the compositions of this invention can be formulated and administered to reduce the symptoms associated with organ transplant rejection, or non-tolerance of an organ transplant. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the an immunosuppressive therapy in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art

A composition of the invention can be administered to a subject in need thereof. Subjects in need thereof can include but are not limited to, for example, a mammal such as a dog, a cat, a cow, a horse, a rabbit, a monkey, a pig, a sheep, a goat, or a human.

A composition of the invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the invention (e.g., that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the invention. Single unit dosage forms suitable for oral administration, such as, but not limited to, tablets, capsules, gel-caps, caplets, or powders, that are adapted for sustained release are encompassed by the invention.

The dosage administered can be a therapeutically effective amount of the composition sufficient to result in treatment of organ transplant rejection or non-tolerance, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

Administration of an immunosuppressive therapy is not restricted to a single route, but may encompass administration by multiple routes. Multiple administrations may be sequential or concurrent. Other modes of application by multiple routes will be apparent to one of skill in the art.

### Kits of the Invention

An aspect of the invention provides a kit for determining donor reactive T-cell frequency in a post-transplant sample comprising: (a) a sample container; and (b) donor reactive T-cell receptor beta gene sequences, wherein each donor reactive T-cell receptor beta gene sequence is determined from T-cells from the recipient that proliferate in response to PBMCs from an organ donor.

An aspect of the invention provides a kit for determining recipient reactive T-cell frequency in a post-transplant sample comprising: (a) a sample container; and (b) recipient reactive T-cell receptor beta gene sequences, wherein each recipient reactive T-cell receptor beta gene sequence is determined from T-cells from the donor that proliferate in response to PBMCs from an organ transplant recipient.

In one embodiment, the kit further comprises an antibody to CD4. In one embodiment, the kit further comprises an antibody to CD8.

An aspect of the invention provides a kit comprising: (a) a urine sample container where DNA in the urine sample is preserved; and (b) donor reactive T-cell receptor beta gene sequences, wherein each donor reactive T-cell receptor beta gene sequence is determined from T-cells from the recipient that proliferate in response to PBMCs from an organ donor.

An aspect of the invention provides a kit comprising: (a) a urine sample container where DNA in the urine sample is preserved; (b) recipient reactive T-cell receptor beta gene sequences, wherein each recipient reactive T-cell receptor beta gene sequence is determined from T-cells from the donor that proliferate in response to PBMCs from an organ transplant recipient.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention, and are covered by the following claims.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

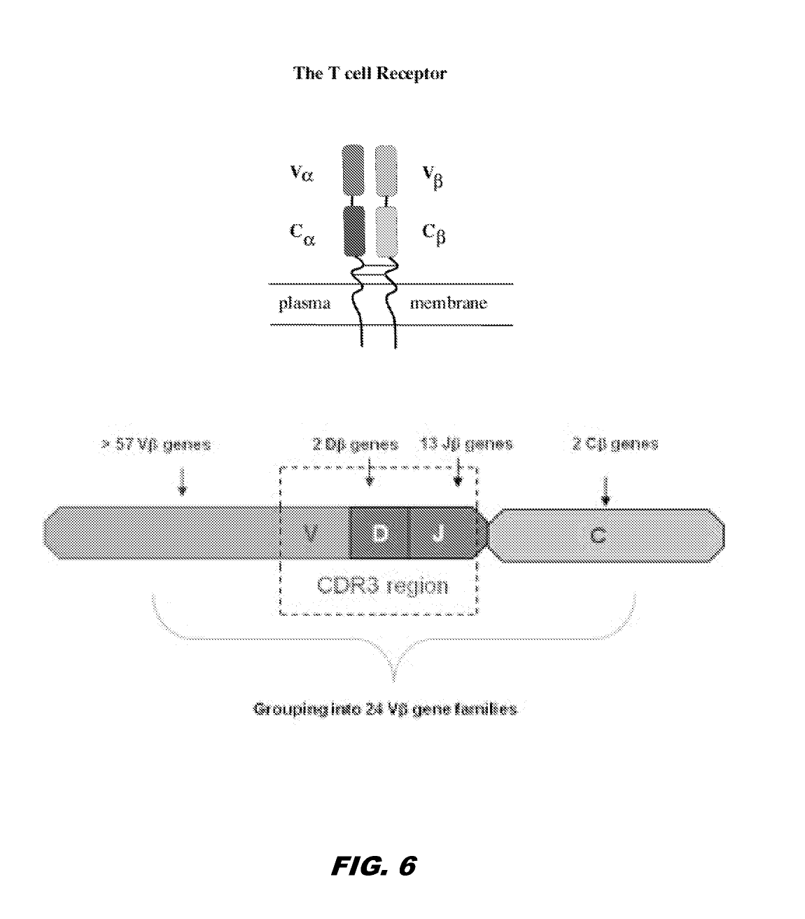

The CDR3 region of the t cell receptor (TCR) serves as a unique “fingerprint” for a given t-cell clone. The peptide specificity of alpha-beta T cells is primarily determined in the third complementarity determining region (CDR3) loops of the alpha and beta chain variable domains. The CDR3 regions are formed by recombination between noncontiguous variable (Vβ), diversity (Dβ) and joining (Jβ) gene segments in the Beta-chain locus. CDR3 sequence diversity is further increased by template-independent addition and deletion of nucleotides at the V/D and D/J junctions (FIG. 6).

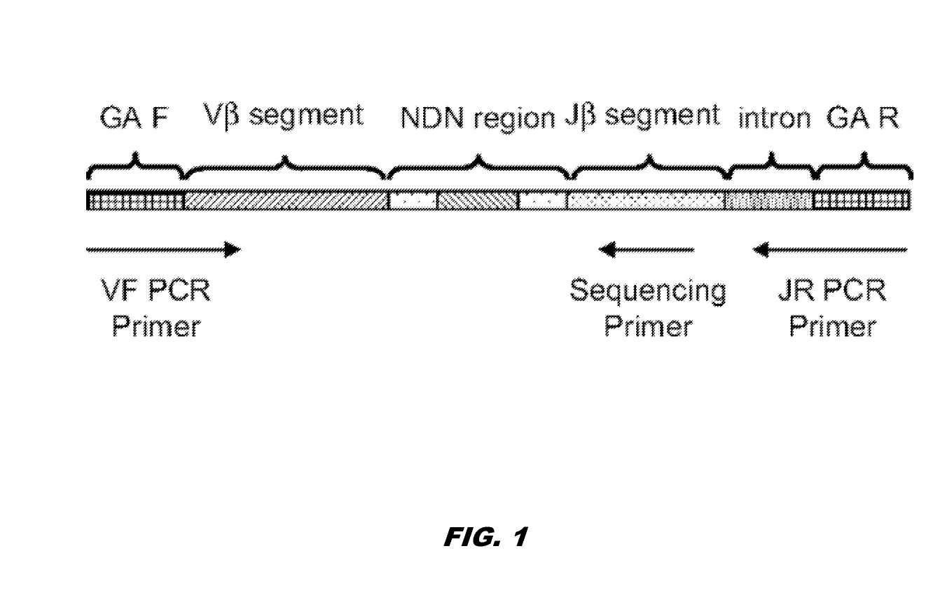

A TCR deep sequencing approach was implemented to identify and track the alloreactive T cell repertoire between a given donor-recipient pair. In a commercially-available technique (ImmunoSEQ; Adaptive™), CDR3 regions of the genomic DNA from T cells were amplified with primers specific for all 45 known expressed Vβ and all 13 Jβ genomic regions of a TCR sequence adapted for solid phase PCR, allowing high throughput sequencing of these regions from millions of T cell clones as well as detection of rare clones (FIG. 1; see Robins H S et al. Blood 2009; 114:4099-4107; and Robins H et al. Journal of Immunological Methods 2012; 375:14-19). High throughput CDR3 sequencing of a CKBMT recipient's donor-responsive T cells in a one-way mixed lymphocyte reaction (MLR) prior to transplant reveals a marked enrichment for donor-reactive TCR sequences, allowing identification of the thousands of TCRs that specifically recognize their organ donor's alloantigens and providing a method of tracking donor-reactive T cells in vivo in the post-transplant period.

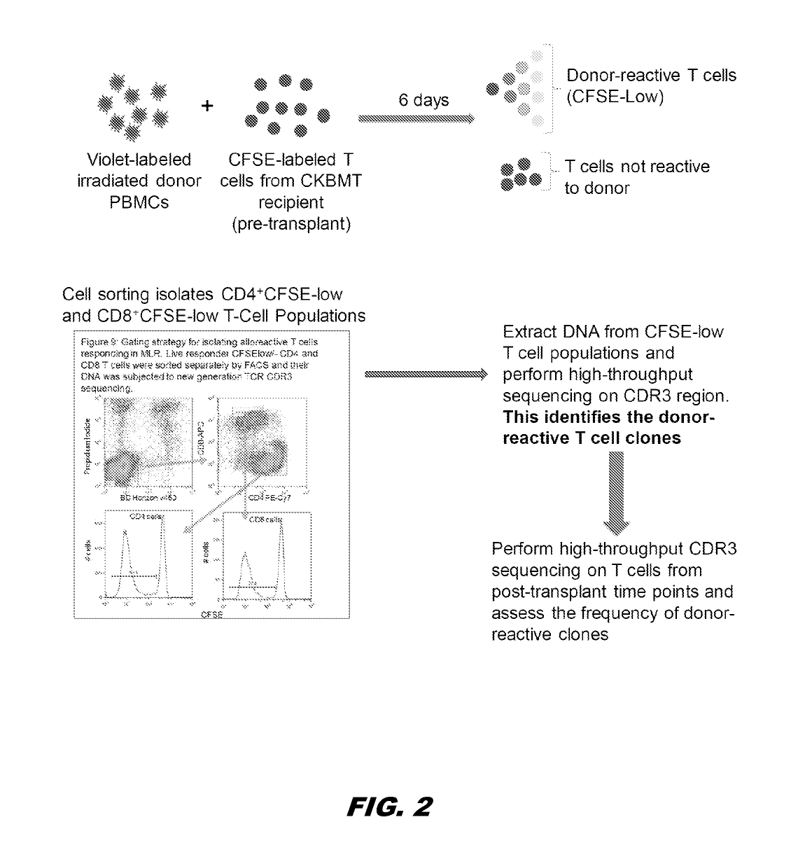

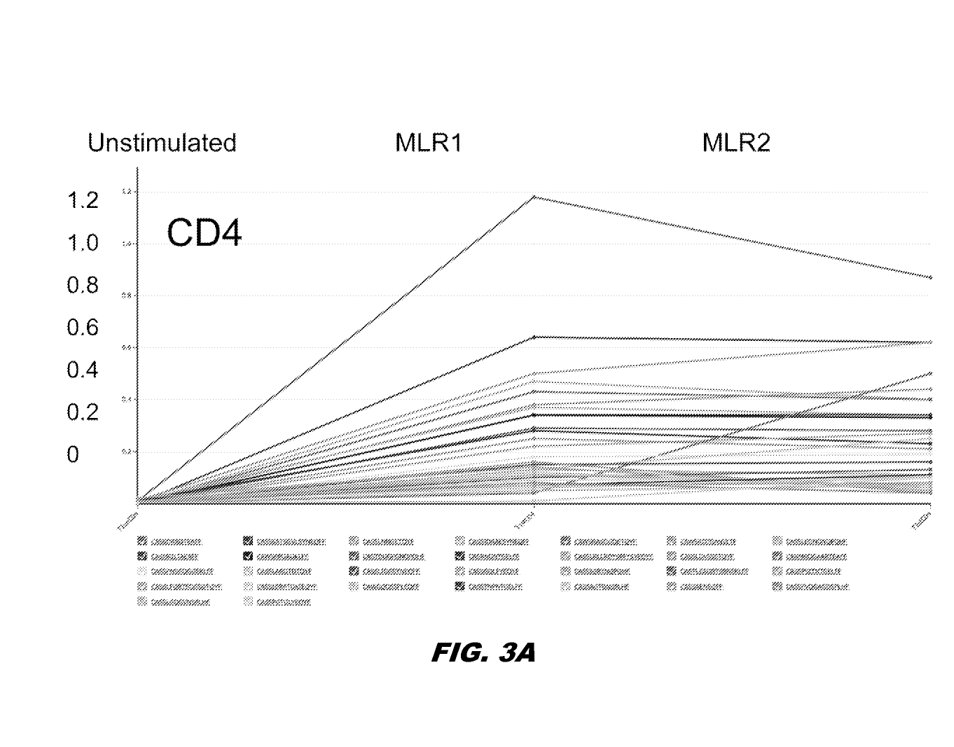

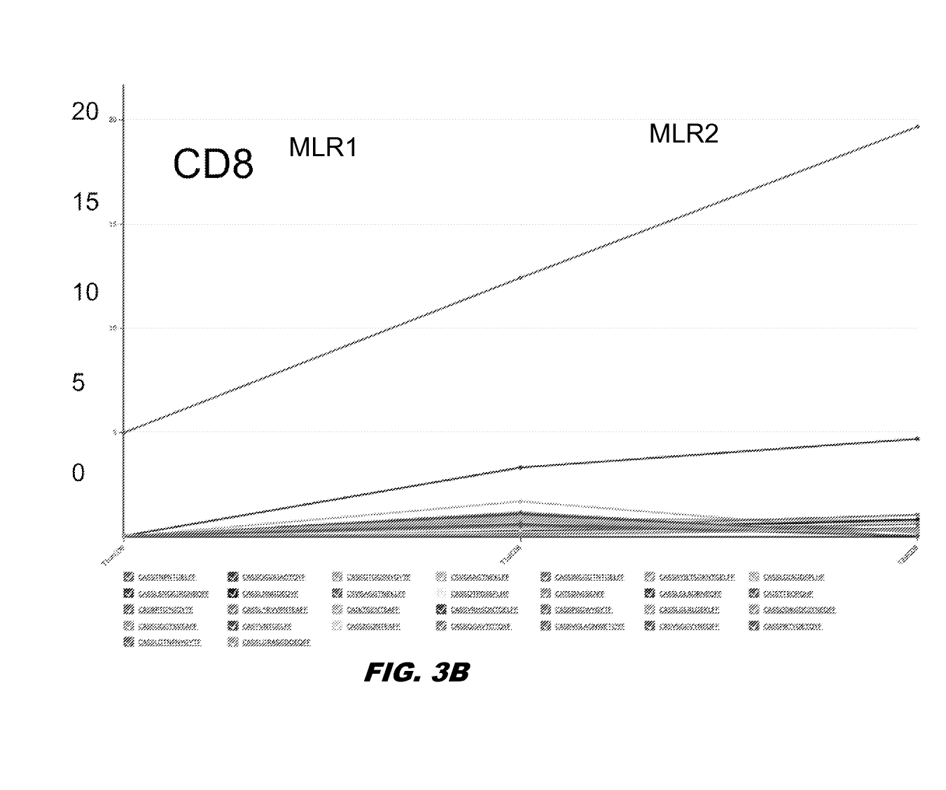

Briefly, donor-reactive T cells are identified by mixed lymphocyte reaction (MLR) between patient T cells and donor antigen-presenting cells, then the TCRs of sorted T cells that have proliferated are sequenced, as indicated by CFSE dye dilution. These donor-reactive T cell sequences are then tracked in transplant patients and their disappearance from blood are correlated with the achievement of transplant tolerance (FIG. 2).

Methods

Pre-transplant patient PBMCs are labeled with CFSE and subjected to 6-day MLR with the donor as stimulator. Stimulator cells are labeled with Horizon Violet Proliferation Dye 450 (VPD450). CD4+ and CD8+CFSE^{low} VPD450-negative cells (i.e., divided T cells) are sorted separately from each MLR (FIG. 2).

DNA is extracted from sorted CFSE^{low} MLR CD4+ and CD8+ T cells (1.0-6.3×10⁶ each), and from fresh, unstimulated CD4+ and CD8+ T cells from the same responder, and CDR3 deep sequencing is performed via Immunoseq at Adaptive™ (FIG. 2).

Expanded donor-reactive TCR sequences can then be tracked in blood and the transplant itself.

Results

In Patient 6 (ITN036 #1; Tolerant patient), sorted CD4 and CD8 cells were compared in the following states:

1) Pre-transplant pt anti-donor MLR (proliferated cells);

2) Pre-transplant unstimulated;

3) 6 months post-transplant unstimulated; and

4) 18 months post-transplant unstimulated.

Without being bound by theory, the frequencies of state 1 would be much greater than the frequencies of state 2, which would be greater than the frequencies of state 3, which would be slightly greater than the frequencies of state 4 (e.g., Frequencies of 1>>>2>>3>4).

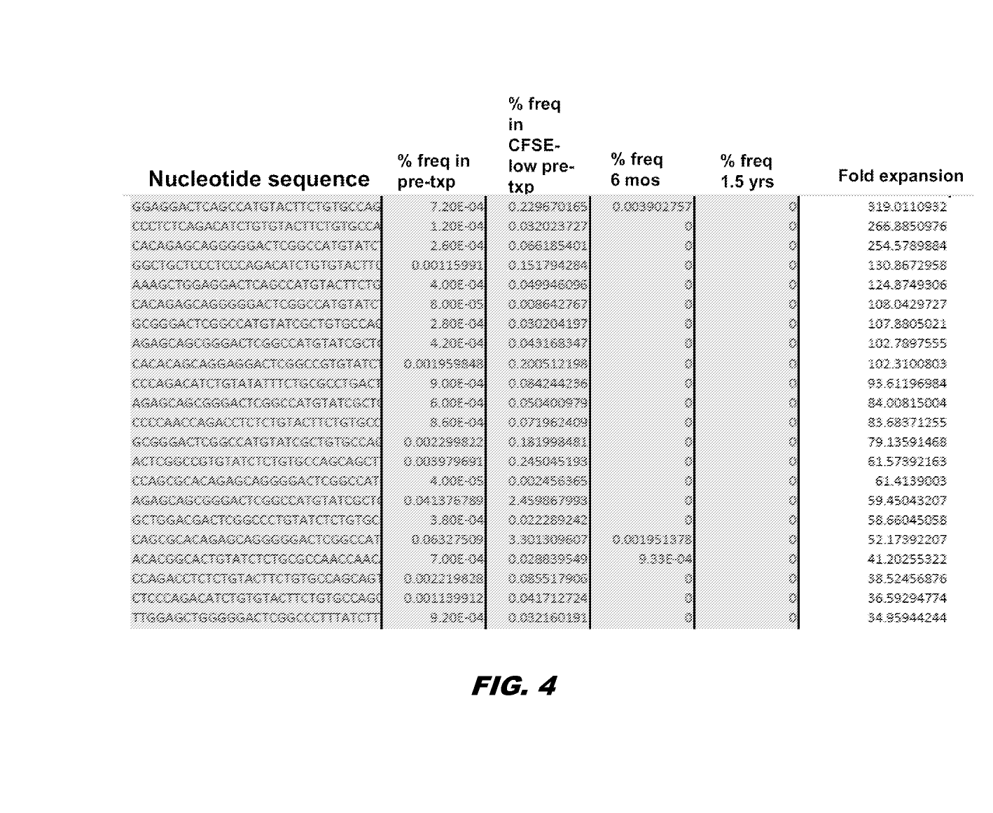

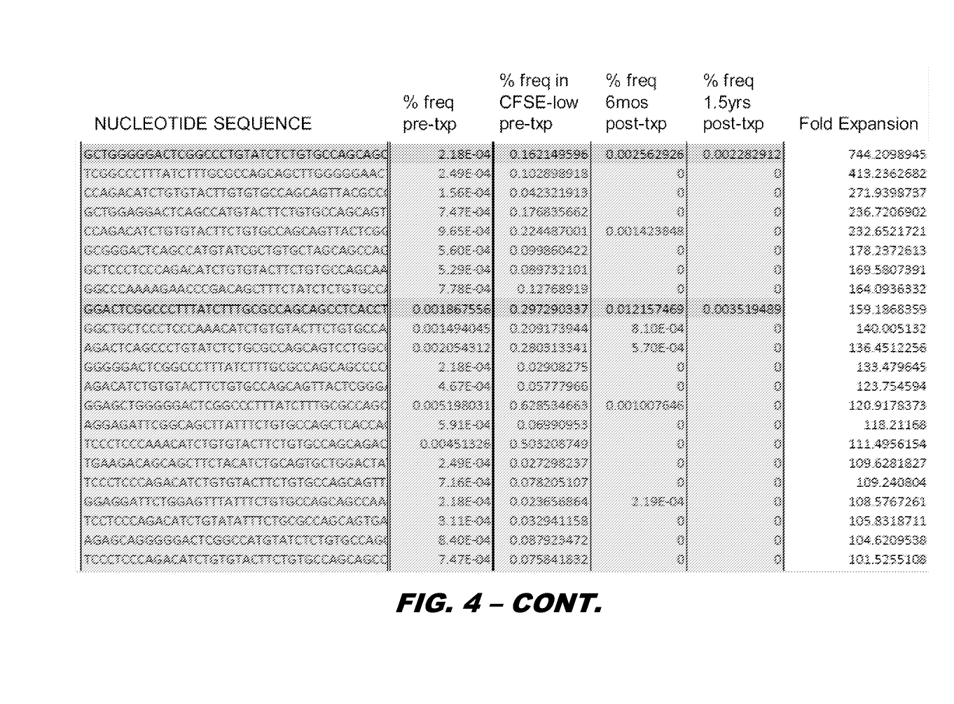

In a study of one tolerant CKBMT patient, thousands of pre-transplant CD4+ and CD8+ T cell clones were identified that were significantly enriched upon exposure to irradiated donor PBMC. Of the clones that were expanded at least ten-fold and were detectable pre-transplant, only 22/164 (13.4%) CD4+ and 10/64 (15.6%) CD8+ clones were still detectable at 1.5 years post-transplant (FIG. 4).

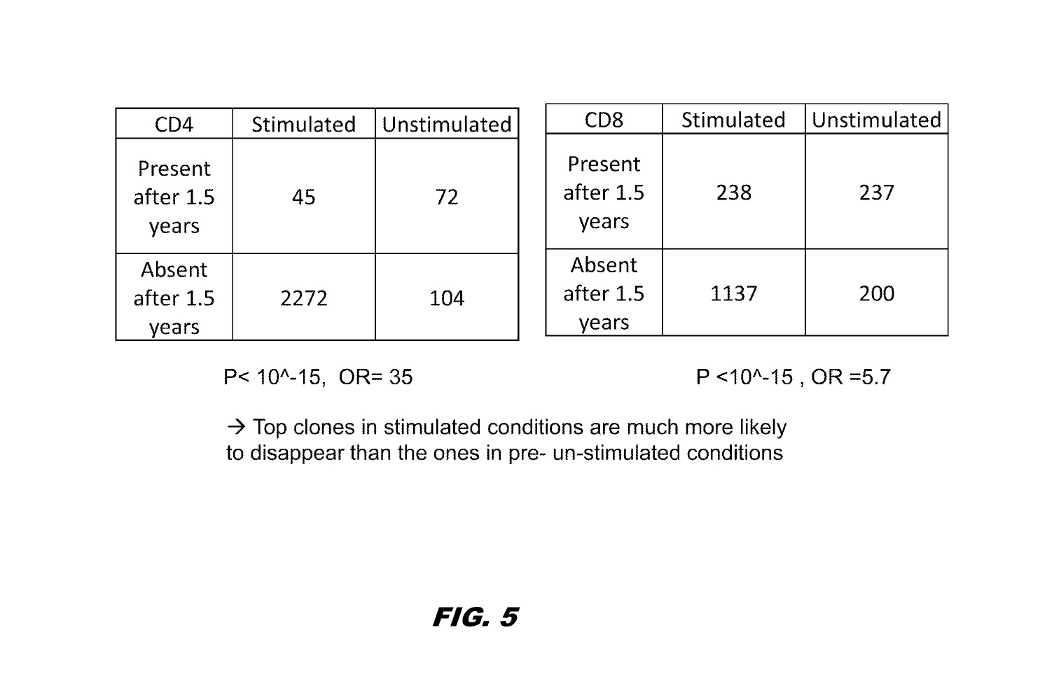

To ensure that the observed disappearance of donor-reactive clones was not due to an inadequate sensitivity to detect low-frequency clones, the rate of donor-reactive clone disappearance was compared with the rate of non-donor reactive clone disappearance. Donor-reactive clones were much more likely to disappear after CKMB transplant than non-donor-reactive clones (OR=35 and 5.7 for CD4+ and CD8+ clones, respectively; p<10⁻¹⁵ for each) (FIG. 5).

Conclusion

High-throughput sequencing of the CDR3 regions of donor-reactive T cells is a new approach that permits physical identification and tracking of the entire alloresponse. A one-way CFSE MLR can be utilized to isolate a CKBMT recipient's donor-reactive T cells prior to transplant. High throughput CDR3 sequencing of a CKBMT recipient's donor-reactive T cells prior to transplant allows identification of TCR clones that specifically recognize his/her organ donor's alloantigens. These clones can be “tracked” in the post-transplant period. In one ITN study patient, deletion of donor-reactive T cells was associated with tolerance following CKBMT. Deletion of donor-reactive T cells likely plays a significant role in the maintenance of tolerance following CKBMT. Thus, tolerance following CKBMT was associated with deletion of donor-reactive T cells in this patient. Further studies are in progress in additional tolerant patients. This experimental setup can be applied to all types of transplant patients, providing an individualized “footprint” of the alloresponse over time.

### Example 2

### Page 1

*Used MLR media with 5% hu-serum

| | |
|---|---|
| 1. | Harvested HM PBMCs and Tcell depleted |
| | HM Tcell yield: 39 million (T2) |
| 2. | Thaw stims: 60 million PBMC = 22/10/21/14 = 30 million |
| | 4 mL PBS, 1:40 dil |
| | Thaw second set responders: 20 million HM Tcells (time 1) |
| | 50/37/40/44 7/26/12 |
| | 5 mL MLR, 1:10 dil = 21 million Tcells - |
| | Label stims with violet dye. |
| 3. | Count stims (after label with violet dye): 12.3 million cells |
| | 5 mL, 1:10 → thawed another 2 vials: 1 mL, .25 |
| | Combined stim count: 25 million → 12.5 mL |
| 4. | Tcells T1: 32/24/20/20 = 12 million → 6 mL |
| | T2: 20/25/20/27 = 12.5 million → 6.25 mL |

### Page 2

CFSE MCR Take down C6

Tcell thaw: 9.75 million → resusp 900 μl facs.

| | |
|---|---|
| (pre wash) | add 90 μl α CD4 |
| | 90 μl α CD8 |

```
          1:40 0.65 mL
T₁ + stim = 98 × 0.65 × 40 × 10⁴ = 25.5 million → resusp in 2.5 mL each
T₂ + stim = dssume the same amount
            add 250 μl α CD4
            250 μl × CD8
```

| | |
|---|---|
| For sort: resusp # 7 & 8 in 1.3 mL | → filtered thru strainer prior |
| | to running |

#9: in 0.5 mL

### Page 3

Pt. 1 CFSE MLR Set Up

```
Pre-txp PBMC: 30.8 million (expected yield = 90 million) → 9 vials
10 mill each thawed
Resusp 124 μl MACS buffer
add 31 μl biotin ab cocktail
10 min
add 93 μl MACS buffer
add 62 μl α biotin microbeads
15 min
Count T cells: 15.5 million → resusp 15.5 mL PBS
(2 mL, 1:10) add 62 μl CFSE mixture
Count donor stims (violet): 22.5 million → resusp 11.25 mL
      5 mL, 1:30
Count CFSE T cells: 10.3 million → resusp 5.15 mL
      3 mL, 1:10
10 wells + 1 well CFSE Ts alone → (300,000)
24 well plate
1 million stims + 1 mill resp./well
Total vol = 1 mL/well
```

### Page 4

### Pt. 1 CFSE MLR Take Down

| PBMC Pre-txp: (expected | 64/67/82/66 | = 55.8 million | |
|---|---|---|---|
| 70 mill) | | | |
| yield 6 mos: (expected 40 mill) | 33/34/32/35 | = 26.8 million | |
| 1.5 yr = (expected 40 mill) | | = 16.2 million | |
| Resusp: | | | |
| Pre txp: 240 μl | 6 mos: 120 μl | 1.5 yr.: | 80 μl |
| add: 60 μl ab | 30 μl ab | | 20 μl ab |
| 10 min | 10 min | | 10 min |
| Add 180 μl buffer | 90 μl buffer | | 60 μl buffer |
| 120 μl ab | 60 μl ab | | 40 μl ab |
| 15 min | 15 min | | 15 min |

| | |
|---|---|
| Plate Harvest: 14 million → | resuspend 1.4 mL (1400 μl) |
| 2 mL, 1:20 | add 210 μl each CD4 and CD8 |
| Tcell yield: | |
| pre-txp: 4 mL, 1:10 = 19.5 million → | resusp 2 mL add 300 μl each CD4 |
| | and CD8 |
| 6 mos 2 mL, 1:10 = 2.9 million → | resusp 300 μl, add 45 μL |
| | cells looked very unhealthy |
| | clumps when resuspending |
| 1.5 yr = 2 mL, 1:10 = 1.9 million → | resusp 200 μl, add 30 μl each |
| For Sort: resusp → | |
| Pre txp stims: 700 μl | |
| Pre txp unstim: 1000 μl | |
| 6 mos. 150 μl | |
| 1.5 yr. 100 μl | |

### Example 3

### A Novel Method for Tracking Alloreactive T Cells: Evidence for Deletion of Donor-Reactive T Cells in Tolerant Patients Following Combined Kidney and Bone Marrow Transplantation

Abstract

Alloreactive T cells recognize myriads of MHC/peptide specificities, precluding their identification in HLA-mismatched transplantation. Described herein is high-throughput deep sequencing of TCR 0 chain CDR3 regions expanded in a pre-transplant mixed lymphocyte reaction that identified and allowed tracking of donor-reactive T cells post-transplantation. This approach was tested in four combined kidney and bone marrow transplantation patients in a trial aimed at achieving allograft tolerance, and in two “conventional” kidney transplant recipients. Post-transplant reductions in donor-reactive T cell clones was observed only in three CKBMT patients who achieved allograft tolerance. Loss of donor-reactive TCR identified tolerance more specifically than functional assays. In some instances, clonal reduction was specific for donor-reactive clones; in others, the reduction reflected repertoire turnover due to lymphocyte depletion during conditioning. Thus, described here in is the development of a method of tracking donor-reactive T cells and obtained evidence for clonal deletion as a mechanism of allograft tolerance in humans.

Introduction

Induction of donor-specific immune tolerance in organ transplantation can avoid the morbidity associated with chronic immunosuppressive therapy and can prevent chronic rejection, which has been a persistent limitation to the current practice. In the first successful trial of intentional tolerance induction to HLA-mismatched organ donors, ten patients (5 subjects in the Immune Tolerance Network [ITN] NKD03 study; 5 subjects in the ITN 036ST study) received combined kidney and bone marrow transplantation (CKBMT) with a non-myeloablative conditioning regimen. Seven of 10 patients have tolerated their allograft for several years in the absence of any immunosuppressive medication, with the furthest patient now off immunosuppression for 12 years (1, 2). In mice receiving T cell-depleting, non-myeloablative conditioning regimens upon which this protocol is based, durable mixed chimerism is achieved and tolerance is largely mediated by intrathymic deletion of newly-developing T cells in the thymus (3-5). In contrast, patients receiving the CKBMT regimens have shown only transient donor chimerism persisting for less than three weeks (1, 6), suggesting that additional mechanisms besides intrathymic deletion of donor-reactive cells must be involved in maintaining long-term tolerance. In the NKD03 cohort, in vitro proliferative and cytotoxic assays gradually became completely, specifically and persistently unresponsive to the donors (7). Evidence from studies of the NKD03 patients suggested a role for regulation/suppression of donor-reactive T-cells early after transplant as tolerant CKBMT patients had significantly higher intragraft Foxp3 mRNA levels compared with stable kidney transplant recipients on standard immunosuppressive regimens (1), CD4⁺CD25⁺CD127⁻FoxP3⁺ regulatory T cells (Tregs) were enriched in the peripheral blood during the early months, and some in-vitro assays revealed evidence of active suppression of anti-donor reactivity at 6-12 months post-transplant (7). Importantly, though, these studies did not reveal anti-donor reactivity when Tregs were depleted in any patient later than one year post-transplant, raising the possibility that deletional tolerance ensued. This possibility has been supported by the results of limiting dilution analyses in long-term tolerant patients, which showed a pattern consistent with deletion and not with suppression (7). Described herein, is a new assay with which to specifically track donor-reactive T cells. Without being bound by theory, deletion of donor-reactive T cells can play a role in the maintenance of long-term tolerance after CKBMT.

Efforts to assess deletion or expansion of donor-reactive clones in transplant patients have been hampered by two main factors: 1) In-vitro donor-specific T-cell unresponsiveness could represent either anergy or deletion, and there is no known functional assay that reliably distinguishes between the two; 2) The proportion of T cells directly recognizing MHC alloantigens is extremely broad, orders of magnitude larger than that of naïve T cells recognizing particular peptide antigens and comprising up to 10% of peripheral T cell (8, 9). Markers to track the many thousands of T cells recognizing donor HLA antigens in a given patient are not available. To circumvent these barriers, described herein is a deep sequencing approach that was devised to identify and track the alloreactive T cell repertoire between a given donor-recipient pair. In one embodiment, a commercially-available technique (ImmunoSEQ; Adaptive™) was used where TCRβ (TRB) CDR3 regions are amplified with primers specific for all 54 known expressed Vβ and all 13 Jβ regions adapted for solid phase PCR, allowing high throughput sequencing of millions of T cell clones as well as detection of rare clones (10, 11). High throughput CDR3 sequencing of a CKBMT recipient's donor-responsive T cells, as identified by expansion in a one-way mixed lymphocyte reaction (MLR) prior to transplant, can identify donor-specific TCR nucleotide sequences that can then be tracked in the post-transplant period. Described herein are the results of this analysis performed in six subjects: four CKBMT patients (Subjects 1, 2, 4, and 5 in the ITN036ST study) and two kidney transplant recipients on standard immunosuppressive regimens. For the CKBMT patients, the clonal analysis data was compared to the results of infunctional in vitro studies assessing donor-specific responsiveness. A significant reduction of circulating donor-reactive T cell clones occurs after transplantation in tolerant CKBMT patients. Furthermore, clonal reduction appears to be a more specific marker for tolerance than donor-specific unresponsiveness in functional assays and a more sensitive tool for detecting the persistence of donor-reactive T cells.

Results

Reproducible Identification of TCR Recognizing the Same Allogeneic Donor in Peripheral Blood Samples at Obtained at Different Time Points

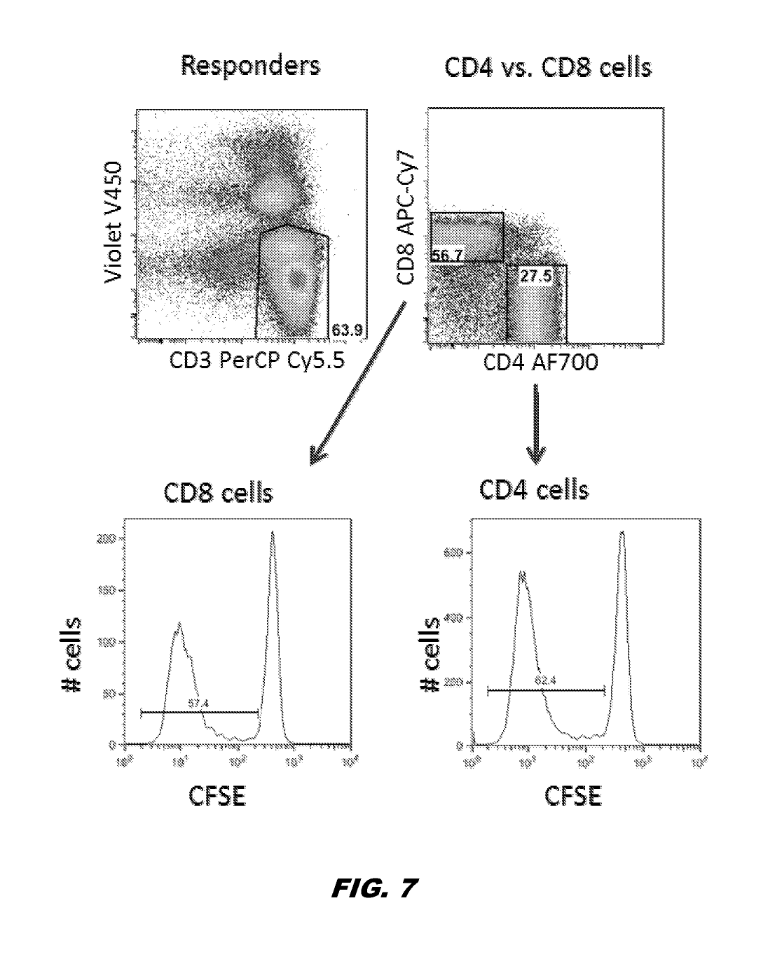

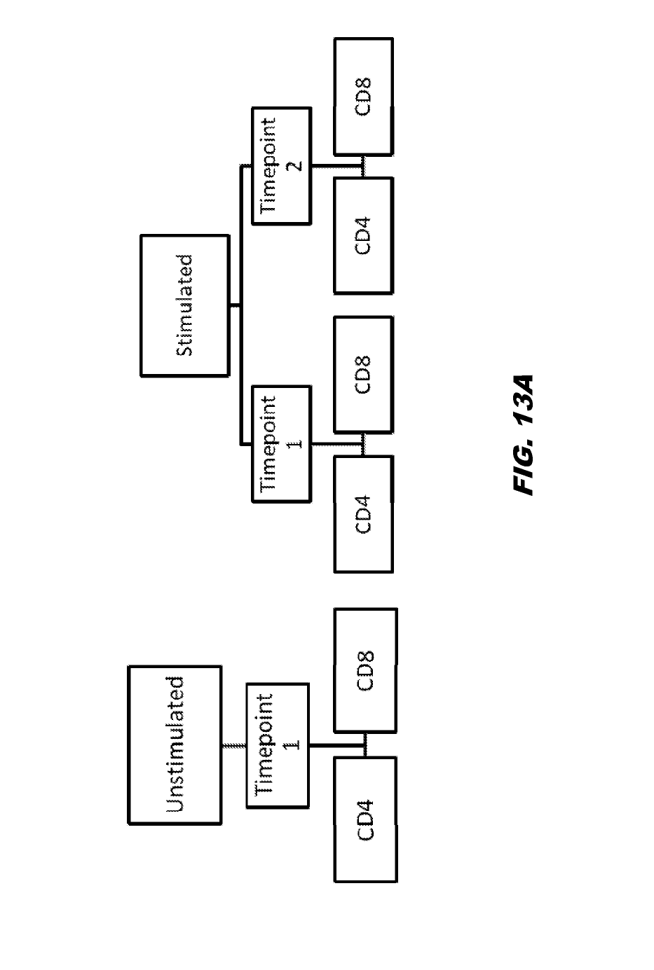

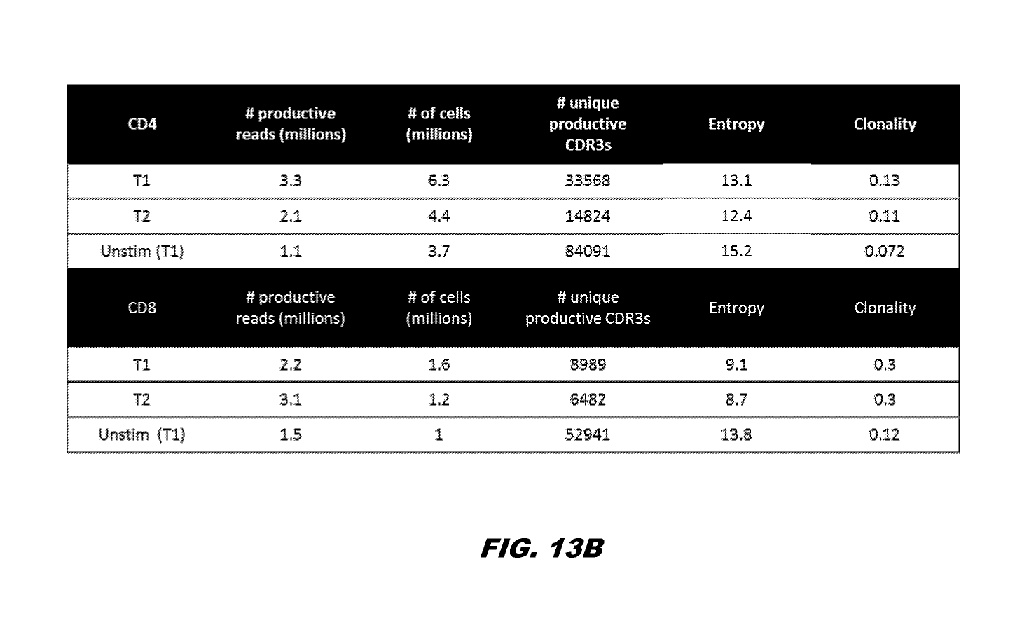

Without being bound by theory, a TCR “footprint” recognizing a given set of alloantigens can be identified by high throughput sequencing of TRB CDR3 regions among T cells dividing in response to these alloantigens in a mixed lymphocyte response (MLR). The same T cell clones responding to a set of alloantigens are detectable in the peripheral blood at multiple time points. Deep TRB CDR3 sequencing was performed on responding T cells from MLRs against the same HLA-mismatched allogeneic donor using PBMCs from repeated blood draws from the same responder at timepoints two weeks apart (FIG. 13A). For each MLR, the responders were labeled with CFSE and the irradiated stimulator PBMCs were labeled with BD violet proliferation dye. After a six-day incubation period, wells were harvested and stained with antibodies against CD3, CD4, and CD8. FIG. 7 illustrates the gating strategy used for cell sorting. Cells were sorted to select for violet negative, CD3-positive, and CFSE-low cells, thereby isolating the recipient T-cells that proliferated in response to alloantigens. These cells were further sorted into CD4-positive and CD8-positive subsets, from which DNA was isolated and subjected to high-throughput CDR3 sequencing. The number of cells from which DNA was extracted along with the number of reads and unique productive sequences obtained for each sample in this study is detailed in FIG. 13B. In all stimulated cell populations, the number of unique sequences in the allostimulated cell populations was reduced, even with the input of greater T cell numbers, compared to unstimulated CD4 and CD8 cells, resulting in a decrease in entropy in the stimulated samples. The entropy of responding CD8 T cells was lower and the clonality higher than that of responding CD4 cells in the same MLR, indicating a more diverse CD4 than CD8 alloresponse.

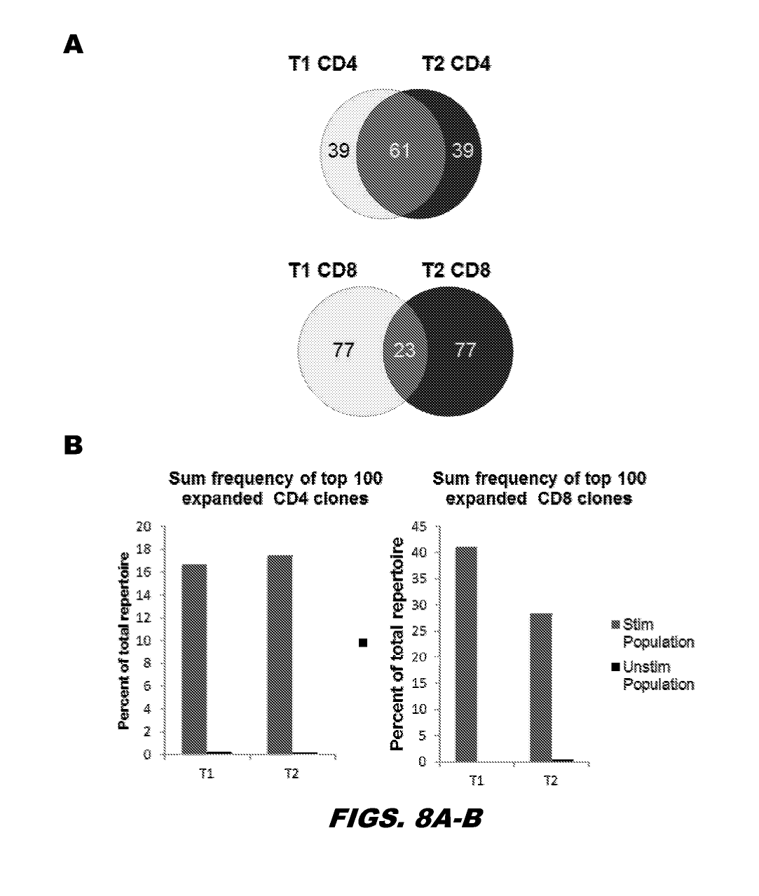

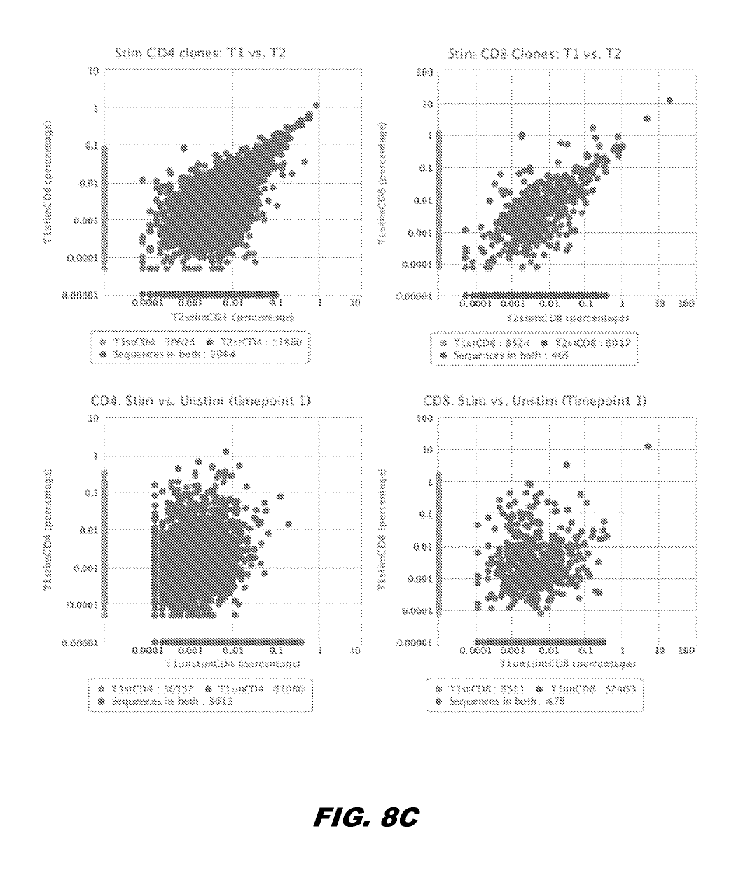

To compare the actual repertoire of the responding T cells in the two different assays, the nucleotide sequences of the 100 alloreactive clones with the highest frequencies in each MLR, excluding those clones which did not expand at least five-fold compared to their frequencies in the unstimulated population were studied (FIG. 8A). Sixty-four percent of the CD4+ clones in the “top 100” of both MLRs subset had identical nucleotide sequences, whereas there was 22% overlap in the CD8+ subset. The top 100 alloreactive CD4 clones comprised roughly 16% of the total stimulated repertoire in each MLR. By comparison, these same clones' summed frequencies comprised only 0.16% of the unstimulated CD4 T cell repertoire. The top 100 alloreactive CD8 clones comprised a relatively higher percentage of the total stimulated repertoire (26.3% in MLR 1, 40.8% in MLR 2), while comprising just 0.23% (T1) and 0.22% (T2) of the unstimulated CD8 repertoire. The lesser degree of clonal overlap in the CD8+ subset can be explained by the greater degree of expansion of a smaller number of individual clones in response to allostimulation, consistent with the higher clonality of the CD8 response as noted above. When the clonal frequencies from each MLR were plotted against each other, a strong linear correlation was observed (FIG. 8C). In comparison, a poor correlation was observed when stimulated clones were plotted against the unstimulated repertoire (FIG. 8B). These results demonstrate that many T cell clones recognizing a given set of alloantigens are reproducibly detectable in human peripheral blood sampled at different timepoints.

Donor-Reactive T Cell Clones are Reduced in the Peripheral Blood of Three Tolerant CKBMT Patients

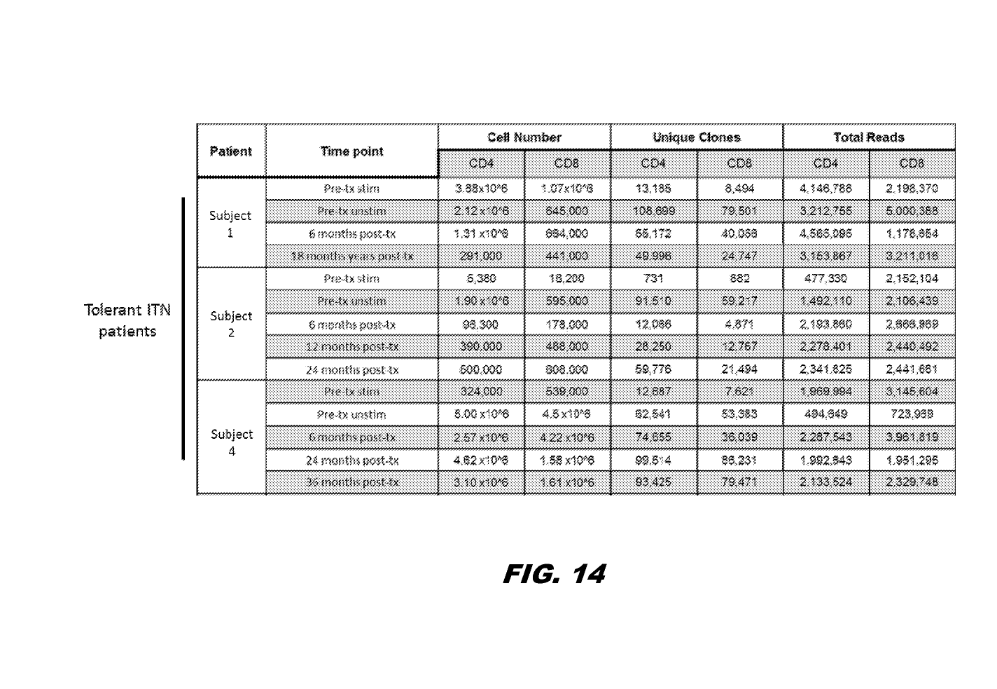

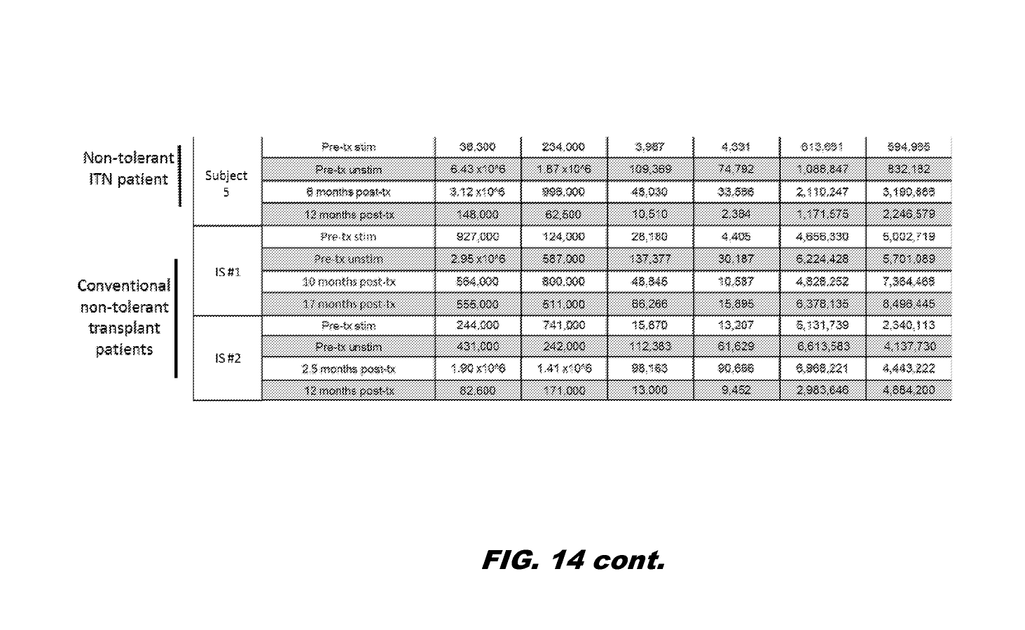

The results presented above suggested that donor-specific clones could be identified prior to transplant in recipient anti-donor MLRs and that they then could be tracked by deep sequencing of TRB CDR3 regions on recipient blood sampled at various times following the transplant. One-way MLRs were set-up using pre-transplant purified T-cells or PBMCs as responders and irradiated donor PBMCs as stimulators in a total of six subjects: four CKBMT recipients (Subjects 1, 2, 4, and 5 from the ITN036ST trial) and two “conventional” kidney transplant recipients on standard immunosuppressive regimens (IS#1 and IS#2). Each anti-donor pre-transplant MLR generated thousands of expanded CDR3 sequences comprising the donor-reactive CD4 and CD8 T cell repertoires. Clones were again defined as donor-reactive if they expanded at least 5-fold in this MLR compared to their frequency in the unstimulated pre-transplant CD4 or CD8 population and showed a frequency ≧10⁻⁴ (0.01%) in the relevant MLR population. These donor-reactive clones were then searched for in unstimulated post-transplant blood samples. Detailed information regarding cell numbers used for sequencing, number of unique sequences, and total reads for each sample is displayed in FIG. 14.

To determine the limit of detection of T cell clones in each of the samples, a power calculation was performed that took into account the number of cells available at each time point. This calculation was essential to prevent a false impression of clonal deletion when cell numbers were limited and the power to detect low-frequency clones therefore reduced.

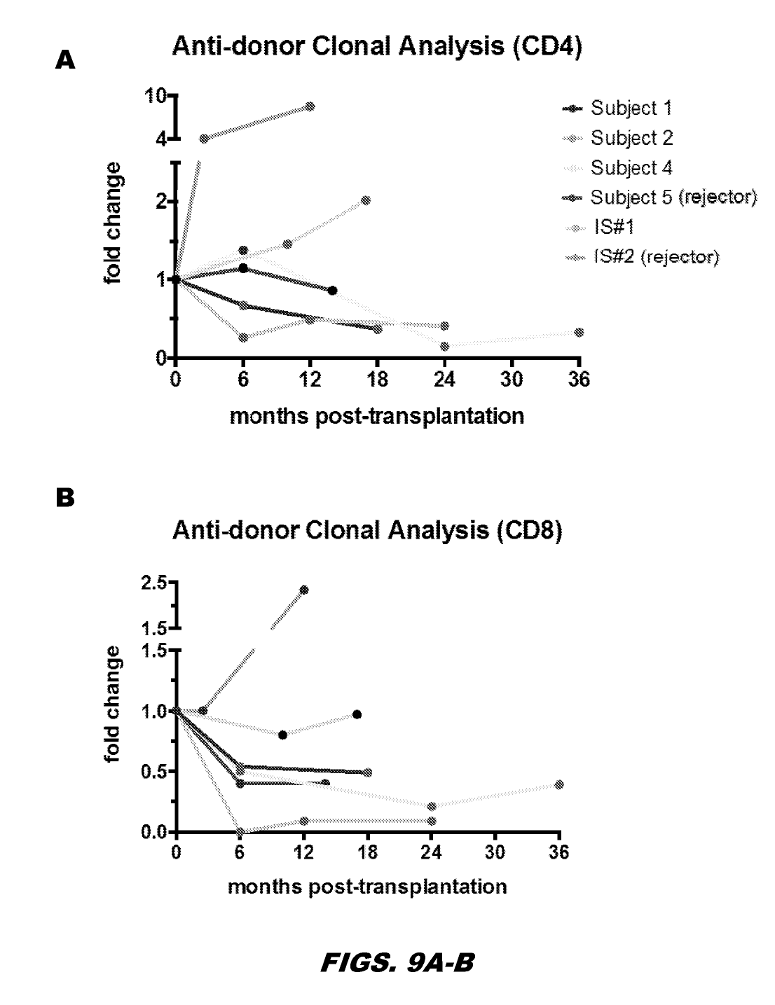

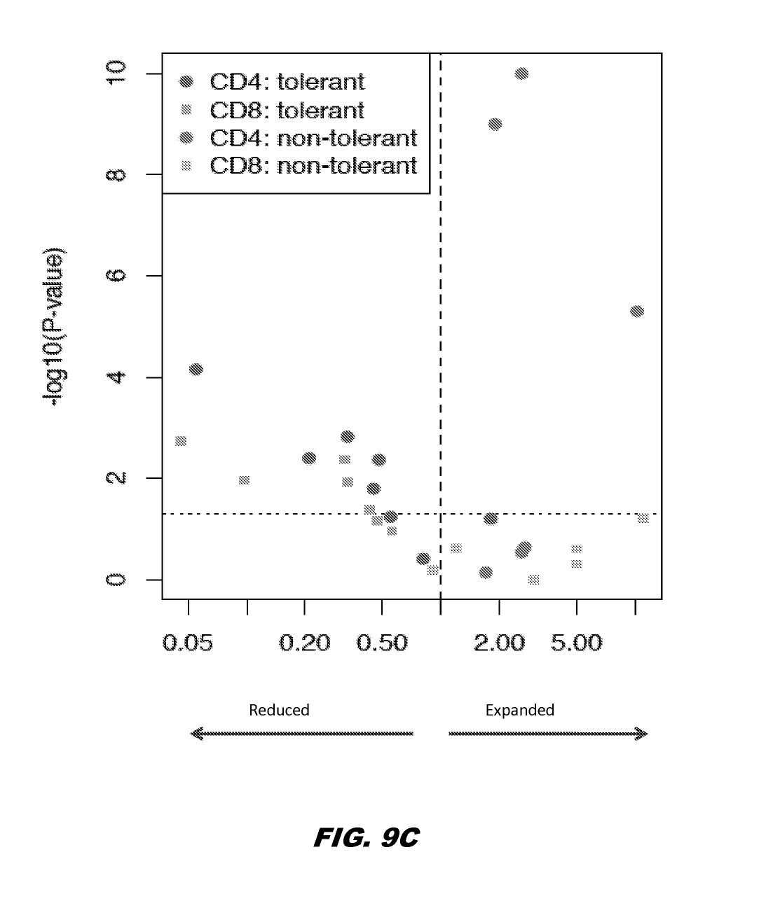

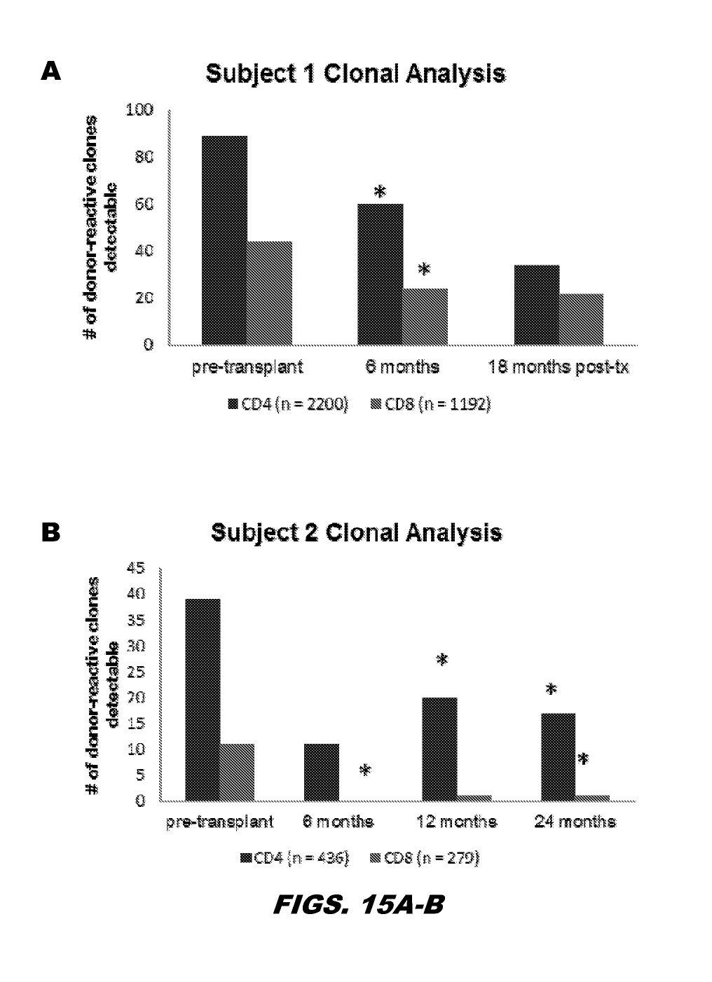

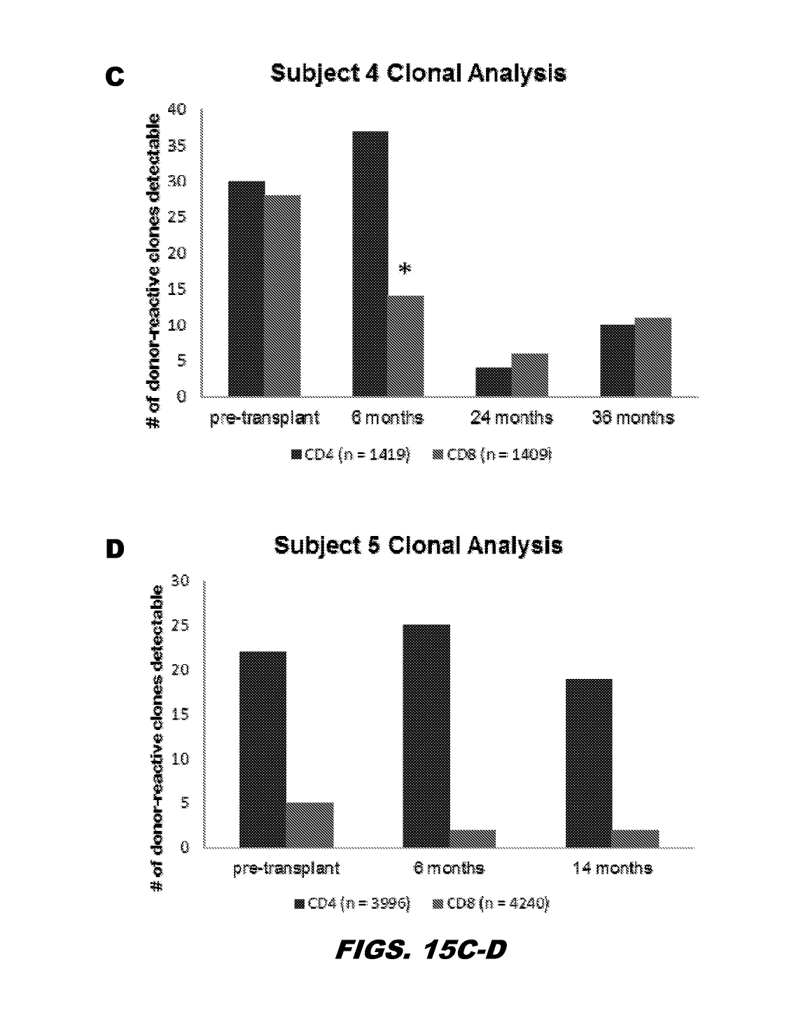

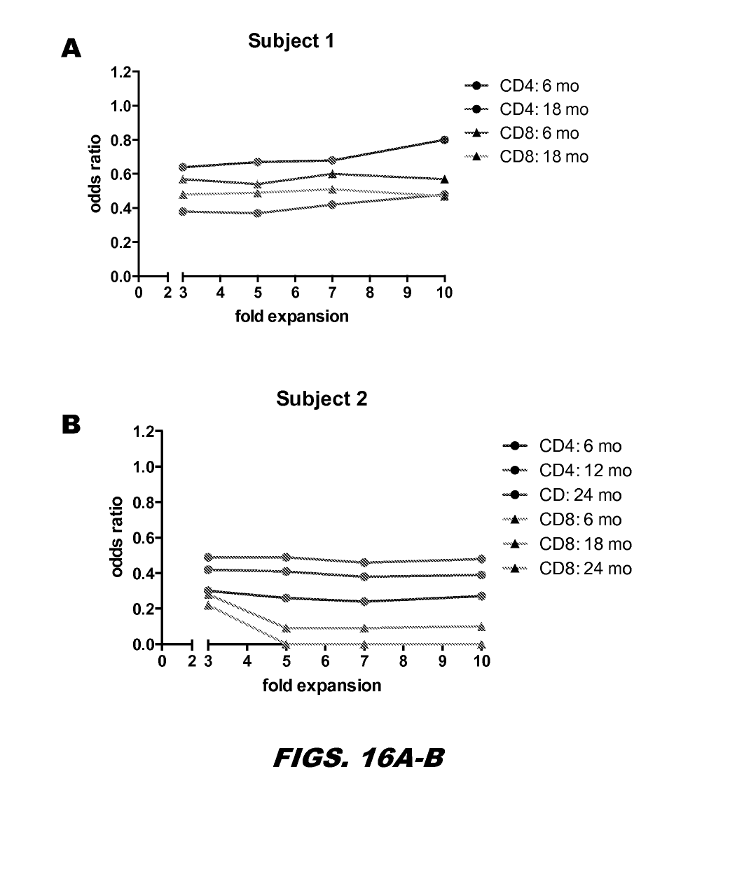

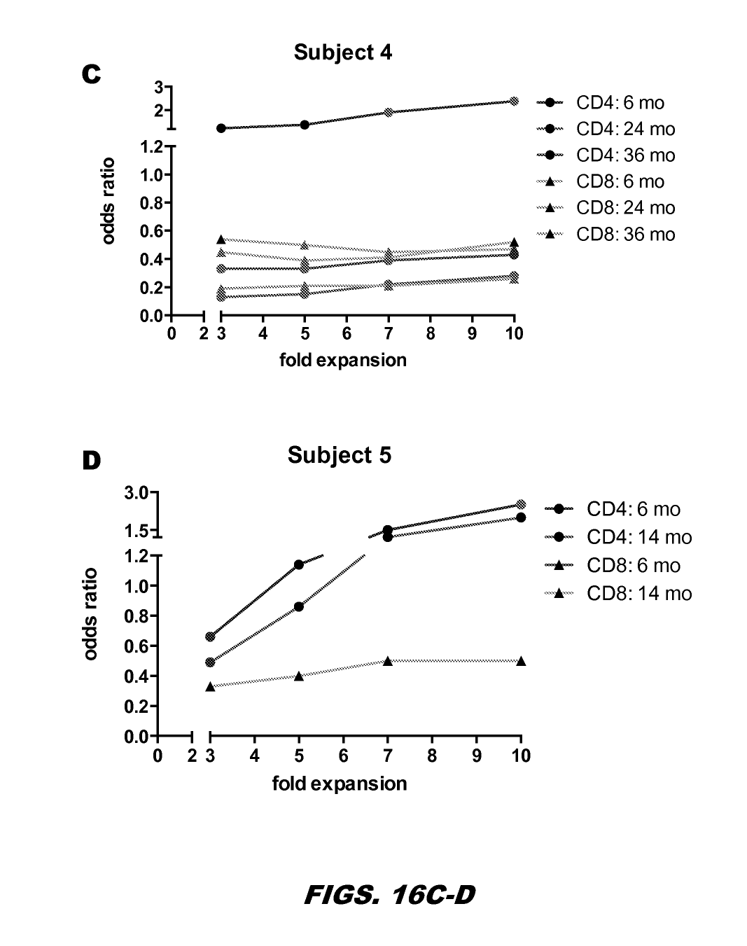

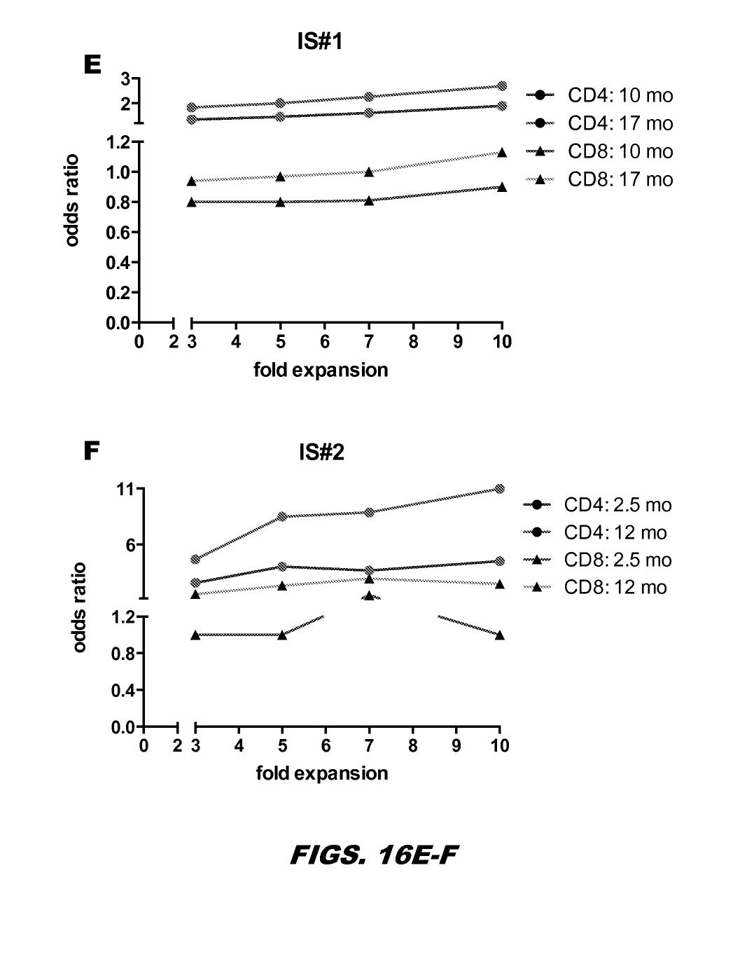

For ITN036 Subject 1, a CKBMT patient whose immunosuppression was discontinued at 8 months post-transplant and who has had stable allograft function for >5 years without any rejection episodes, there was sufficient power to detect clones with a ≧10⁻⁵ frequency (0.001%) at each time point. All CD4 and CD8 clones defined as donor-reactive in the pre-transplant MLR were then examined. 2240 such CD4⁺ clones and 1281 CD8⁺ clones were identified and compared to the number that were detectable in unstimulated cell populations pre- and post-transplant. A significant reduction in the rate of detection of donor-reactive CD4+ and CD8+ clones was observed in the blood at both 6 and 18 months post-transplant (summarized in FIGS. 9A-B; details in FIGS. 15A-D). This analysis was consistent with the possibility that deletion of donor-reactive T cells can contribute to tolerance in Subject 1. Pre-transplant MLR achieved donor-reactive clones at 5-fold expansion as well as using different definitions, ranging from 3-fold to 10-fold expansion, as shown for all subjects in FIGS. 16A-F.

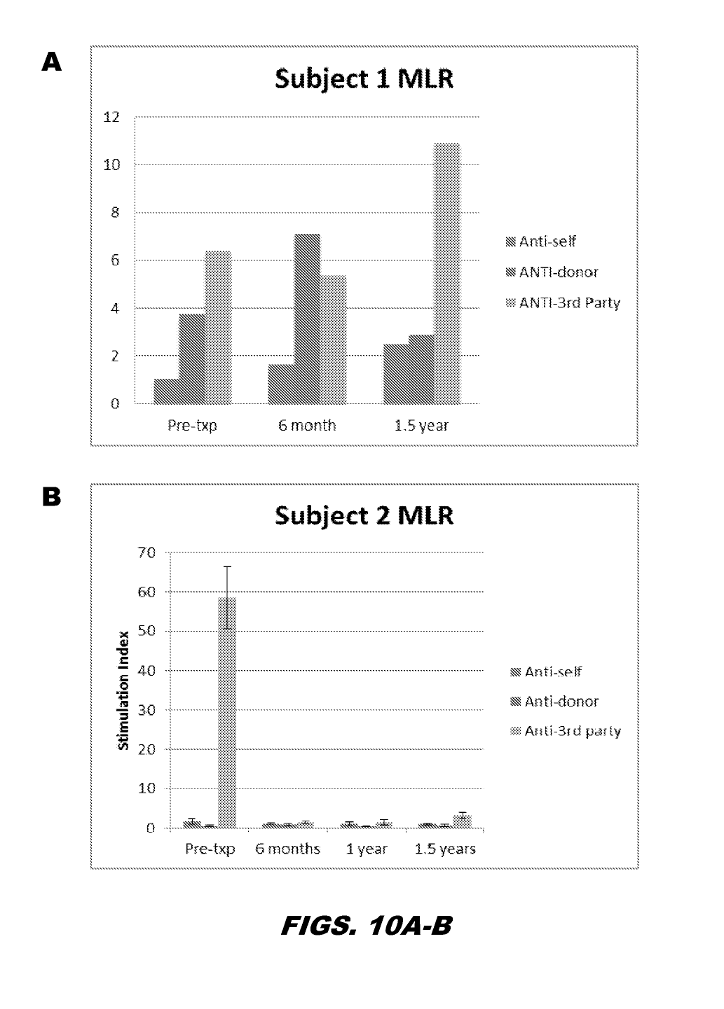

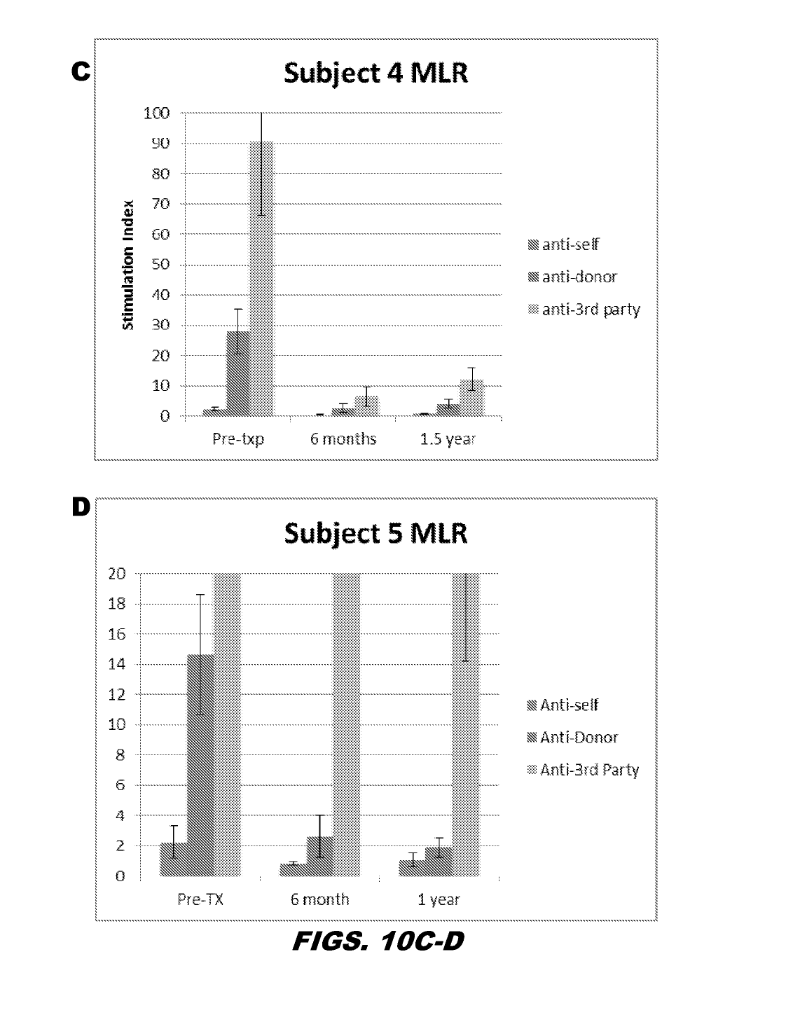

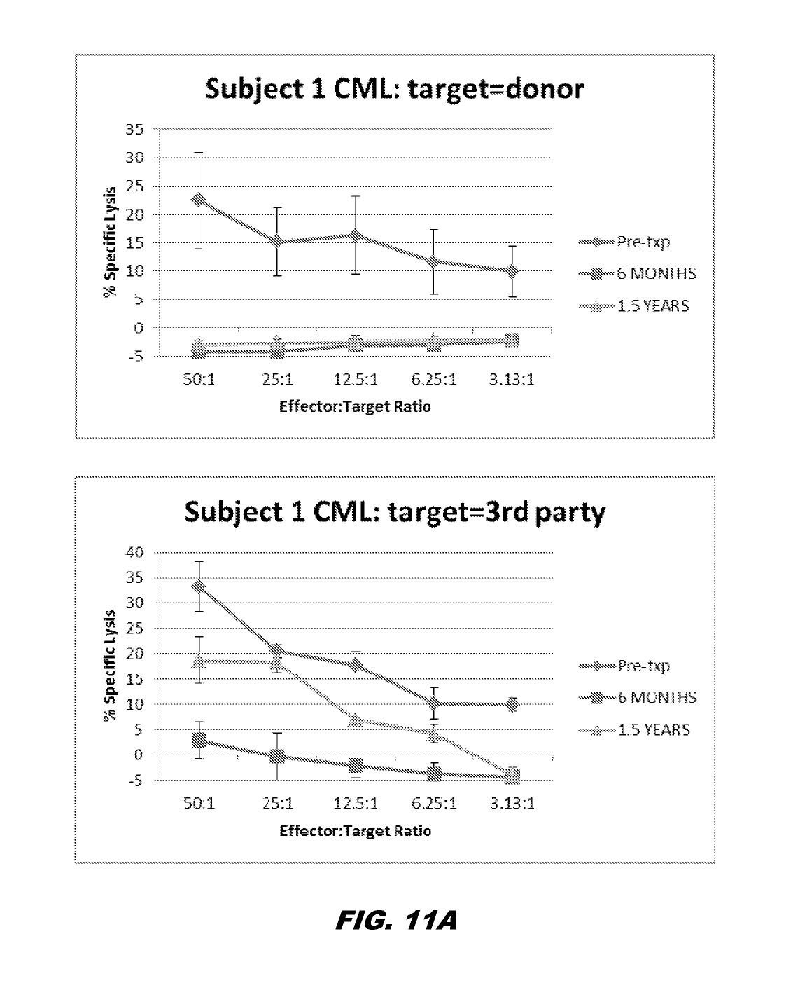

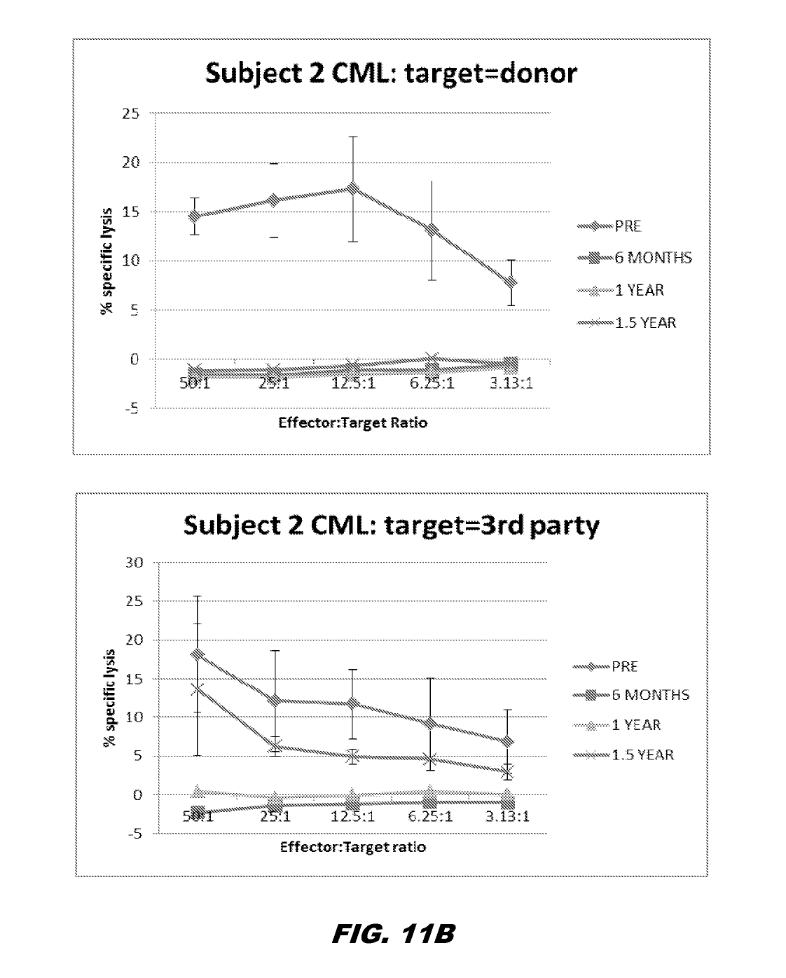

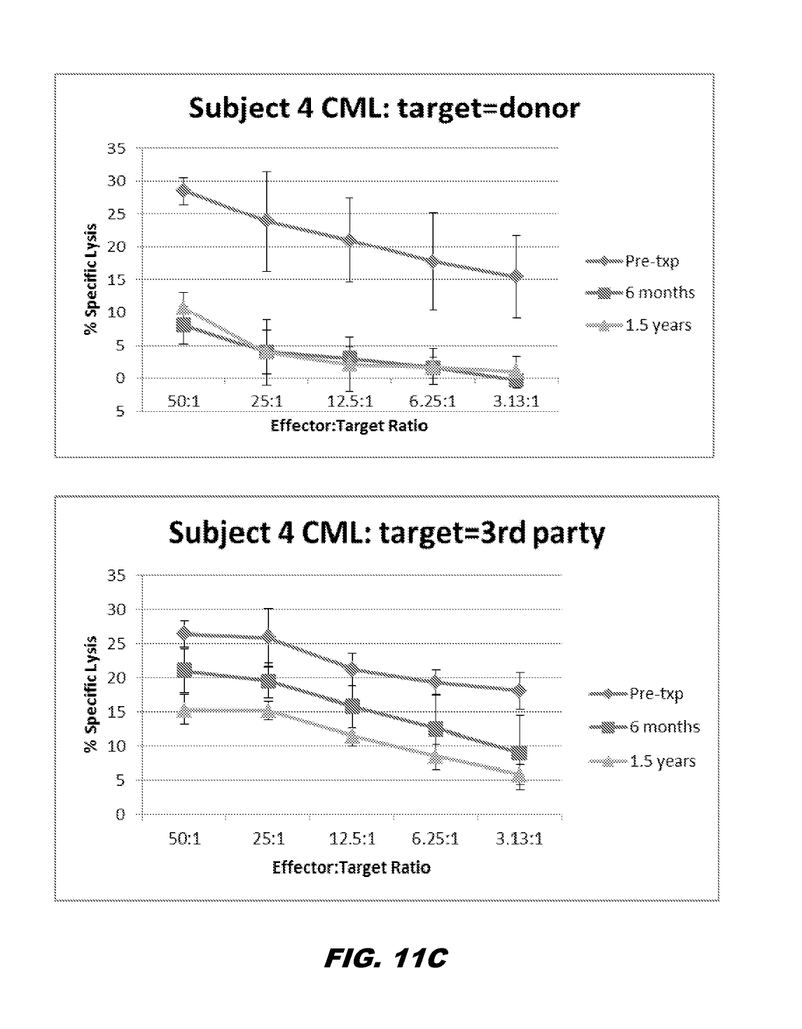

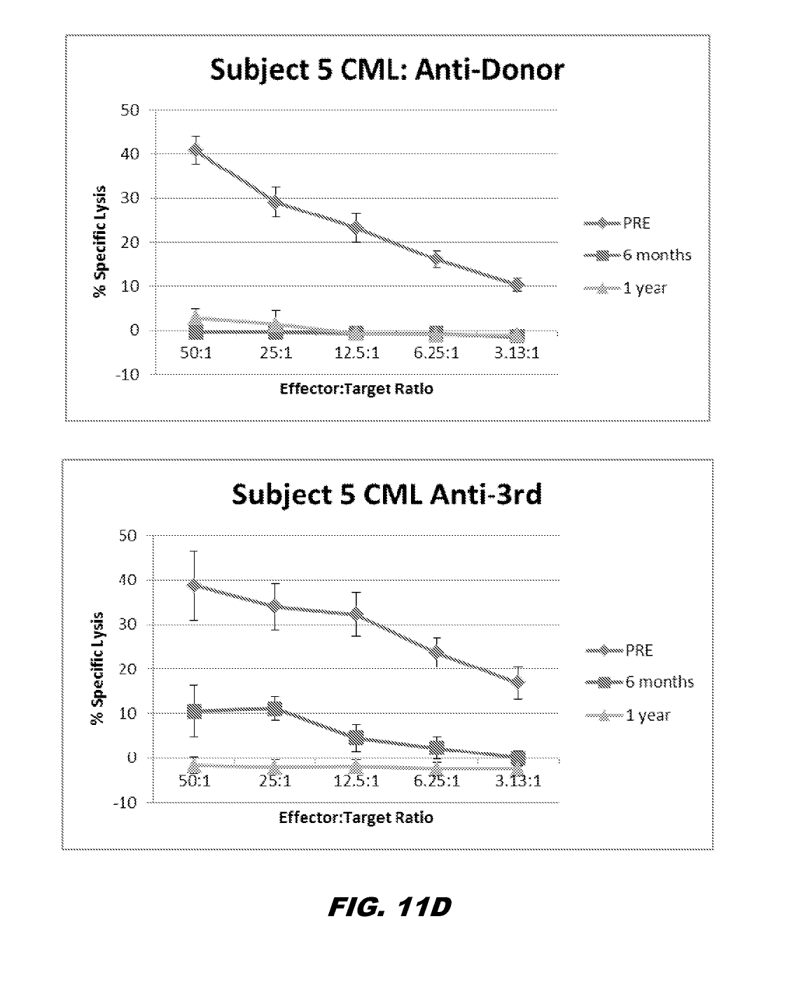

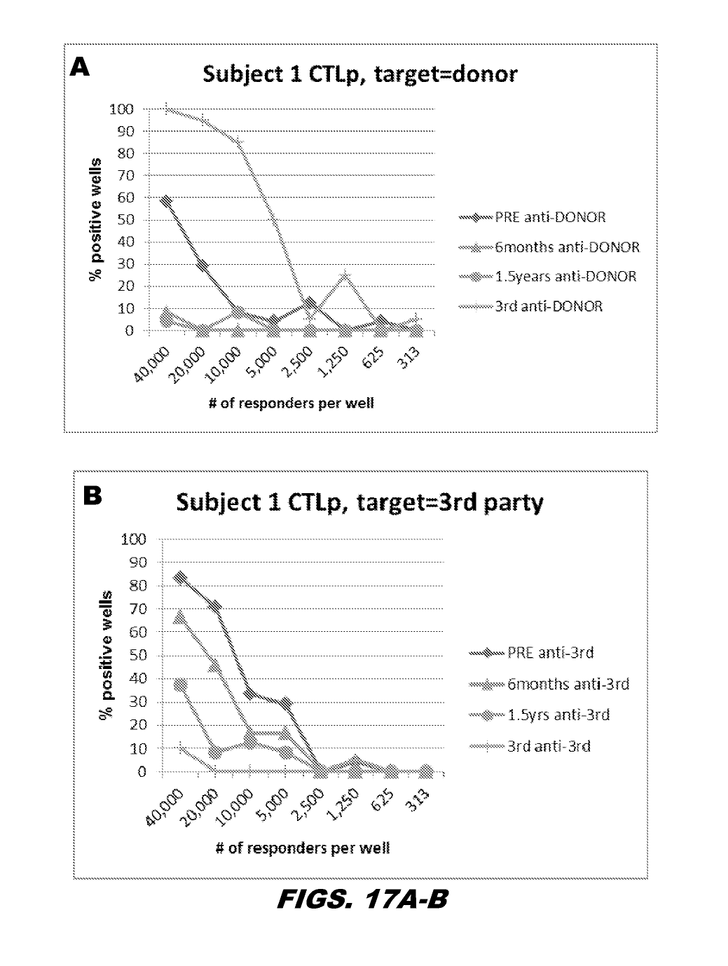

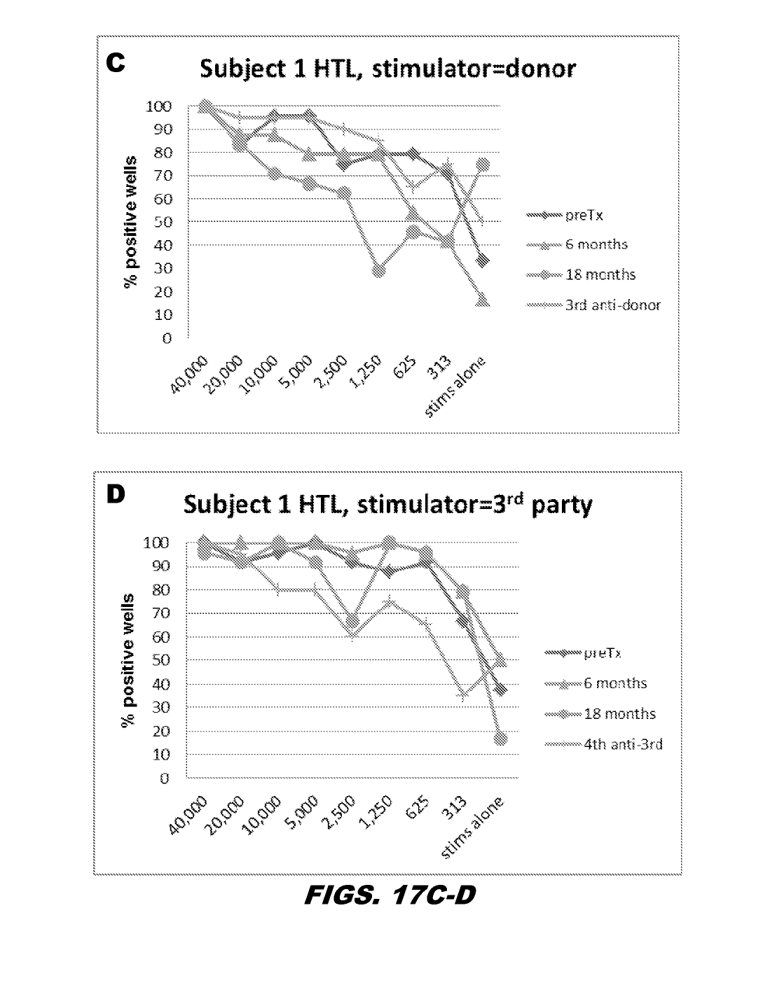

These results were compared to functional assays of anti-donor reactivity at the same time points. The corresponding MLR assay in this subject (FIG. 10A) showed persistent anti-donor reactivity (greater than anti-self) at 6 months that disappeared at 1.5 years. Cell-mediated lympholysis (CML) assays (FIG. 11A) revealed disappearance of anti-donor reactivity at both 6 months and 1.5 years. Clonal analysis therefore appears to be more sensitive than MLR and CML in detecting the presence of residual donor-reactive clones and provides specific information on the level of deletion. Limiting dilution analyses was also performed to quantify cytotoxic T lymphocyte precursors (CTLp) and IL-2-producing helper T lymphocytes (HTL) in functional assays. The CTLp assay (FIGS. 17A-D) showed loss of donor reactivity by 6 months, suggesting that anergy of cytotoxic T-lymphocytes may develop early after transplant before significant deletion occurs. In contrast, the HTL assay (FIGS. 17A-D) revealed measurable anti-donor responses at all post-transplant time points, indicating that, in this subject, the HTL was the most sensitive functional assay for detection of persistent anti-donor reactivity.

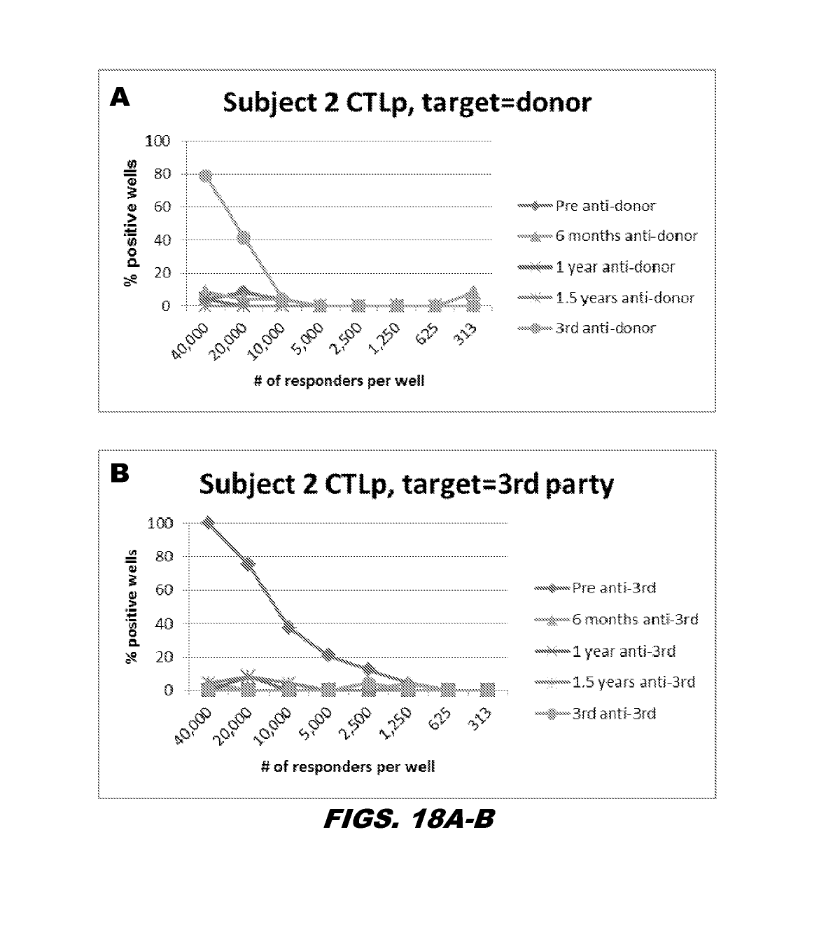

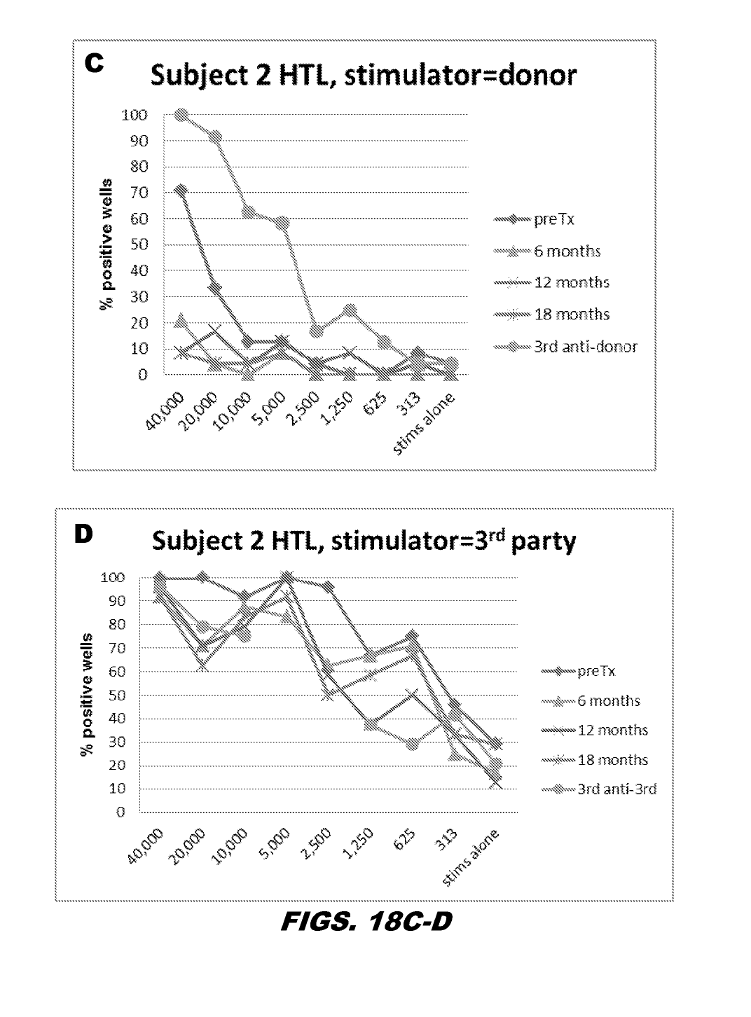

Similar analyses in three additional CKBMT patients were performed, two of whom achieved tolerance. The results are summarized in FIGS. 9A and 9B and details are provided in FIGS. 15A-D, 16A-F, and 18A-D. Subject 2 is currently >5 years post-CKBMT; her immunosuppressive therapy was discontinued at 8 months and she has had normal kidney function with no rejection episodes. Her pre-transplant CFSE-MLR yielded a very weak response, perhaps due to the high degree of HLA matching with her related donor (1/2 mismatch at HLA-A locus; 0/2 mismatch at B, DR, and DQ loci). Both CD4+ and CD8+ donor-reactive clones were significantly less likely to be detectable at all times (6, 12, 24 months) after CKBMT than before (FIGS. 9A-B and FIGS. 15A-D, 16A-F) consistent with deletional tolerance. The corresponding post-transplant MLR studies (FIG. 10B) yielded little if any information about the status of donor-specific tolerance in this patient due to the weak anti-donor reactivity at all time points, including pre-transplant and poor anti-third party control responses post-transplant. CML (FIG. 11B) showed recovery of the anti-3^{rd} party response with an absent anti-donor response at 1.5 years, but could only demonstrate global unresponsiveness at 6 months post-CKBMT. CTLp against donor and 3^{rd} party were undetectable at all time points, and HTL revealed very weak anti-donor reactivity only at the highest responder concentrations (FIGS. 18A-D). Therefore, in Subject 2, TCR analysis was more sensitive for detecting residual donor-reactive CD4 clones than MLR. Overall, the TCR clonal analysis provided evidence for partial but incomplete deletion of donor-reactive T cells that were identified pre-transplant.

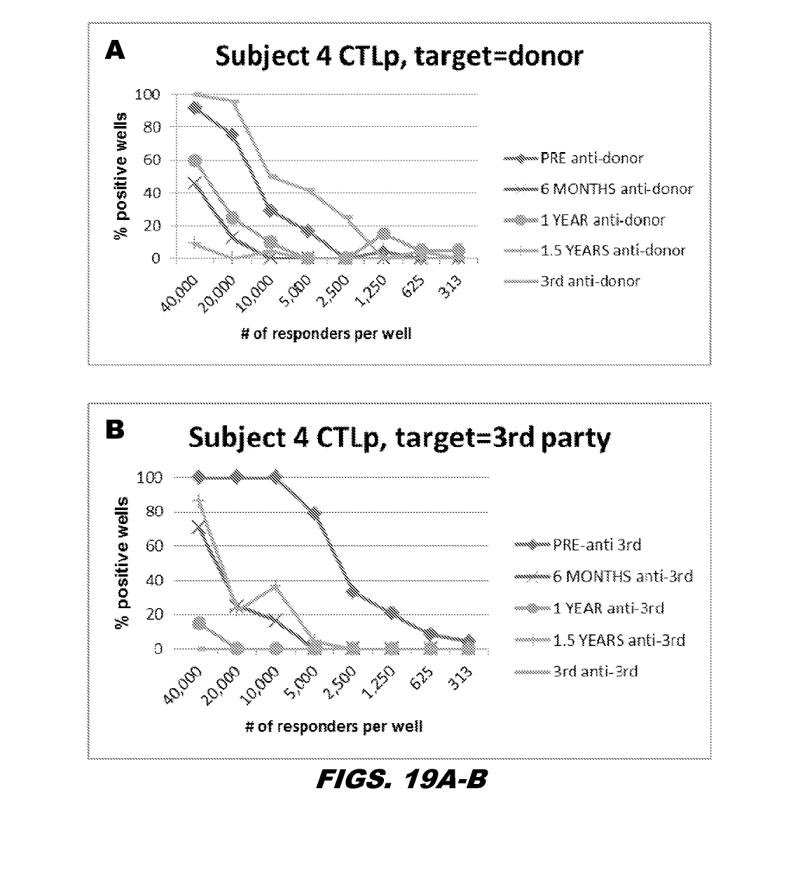

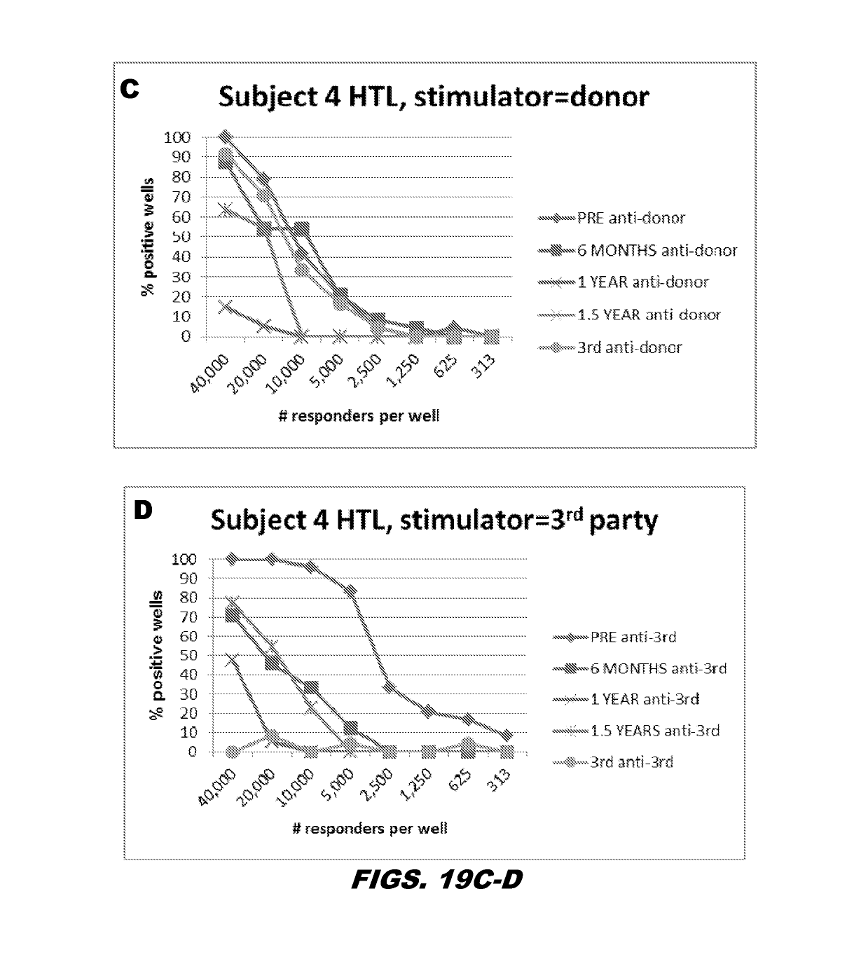

Subject 4 is also showing allograft tolerance more than 4.5 years after CKBMT, with immunosuppressive therapy having been discontinued at 8 months. FIG. 9B and FIGS. 15A-D demonstrate a significant reduction in CD8+ donor-reactive clones at 6 months, with further progression to deletion at 2 and 3 years post-transplant. The donor-reactive CD4 clones showed a slight but non-significant increase in frequency at 6 months post-transplant followed by significant deletion at 2 and 3 years (FIG. 9A and FIGS. 15A-D, 16A-F). In this subject, the MLR assay (FIG. 10C) revealed donor-specific hyporesponsiveness, but not unresponsiveness by 6 months and later. The CML (FIG. 11C) showed small but detectable anti-donor responses at 6 and 18 months. In the CTLp analysis (FIG. 19A-B), the anti-donor response declined over time but remained detectable at the highest responder concentrations. The HTL assay (FIG. 19C-D) revealed anti-donor responses at all post-transplant time points except for one year. Therefore, in Subject 4, all functional assays reveal some degree of persistent anti-donor response, which is in keeping with the clonal analysis showing persistent, gradually reducing, donor-reactive clones.

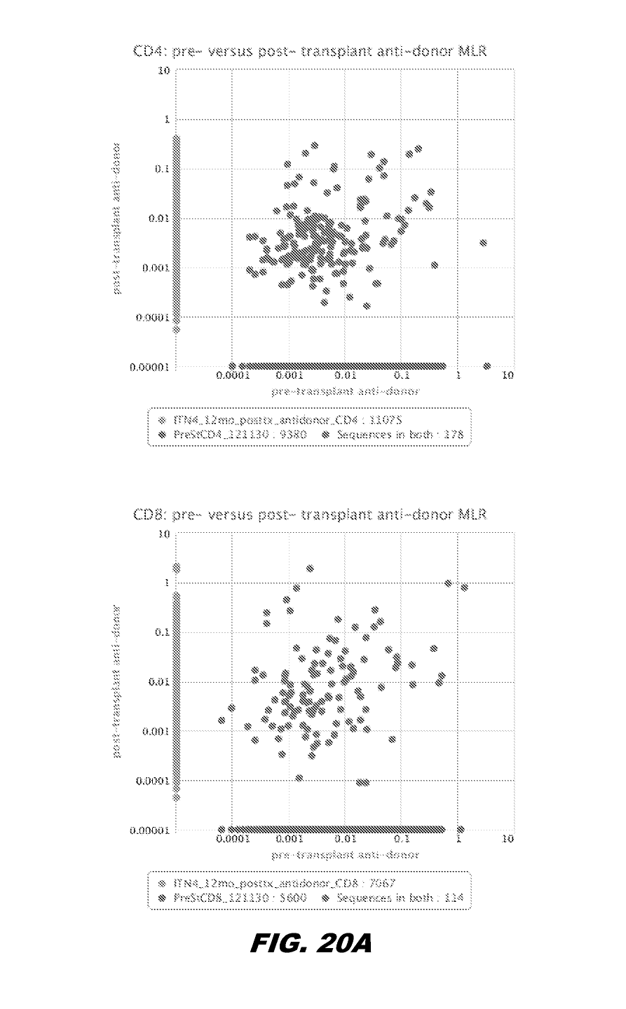

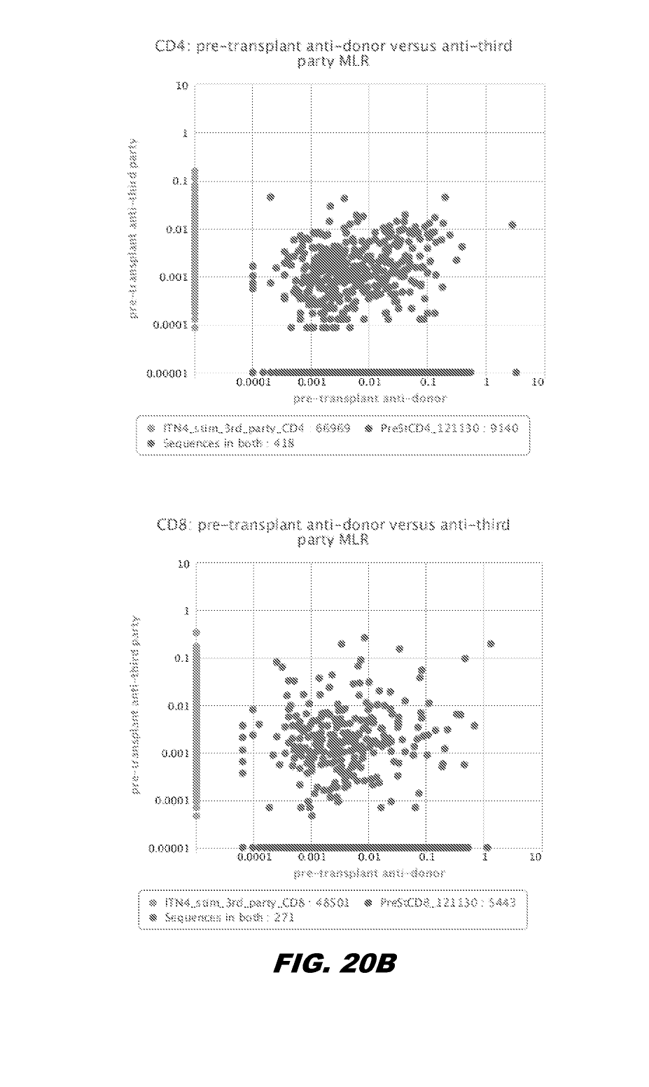

Subject 4 differed from Subjects 1 and 2 and from NKD03 patients (7) in showing persistent anti-donor reactivity in MLR and CML assays, despite marked deletion of pre-existing donor-reactive clones over time. These responses can be mediated by T cells that developed de novo post-transplant rather than the same ones mediating the MLR prior to transplant. To test this possibility, the MLR was repeated with a 1-year sample, sorted the CD4 and CD8 cells that divided in response to the donor, and performed clonal analysis. Only 17 of the CD4 and 14 of the CD8 clones that were among the top 1000 clones responding in the pre-transplant MLR were among the top 1000 in each subset responding post-transplant and the overall poor correlation between the expanded clones in both MLRs is shown in FIGS. 20A-B. This minimal level of overlap was not greater than that among the top 1000 CD4 (n=11) and CD8 (n=18) clones in post-transplant anti-donor and pre-transplant anti-3rd party responses. These data suggested that the post-transplant MLR detected in Patient 4 may largely reflect responses of T cells that developed de novo following the transplant.

Lack of Evidence for Deletion of Donor-Reactive Clones in a CKBMT Recipient Who Failed Tolerance Induction

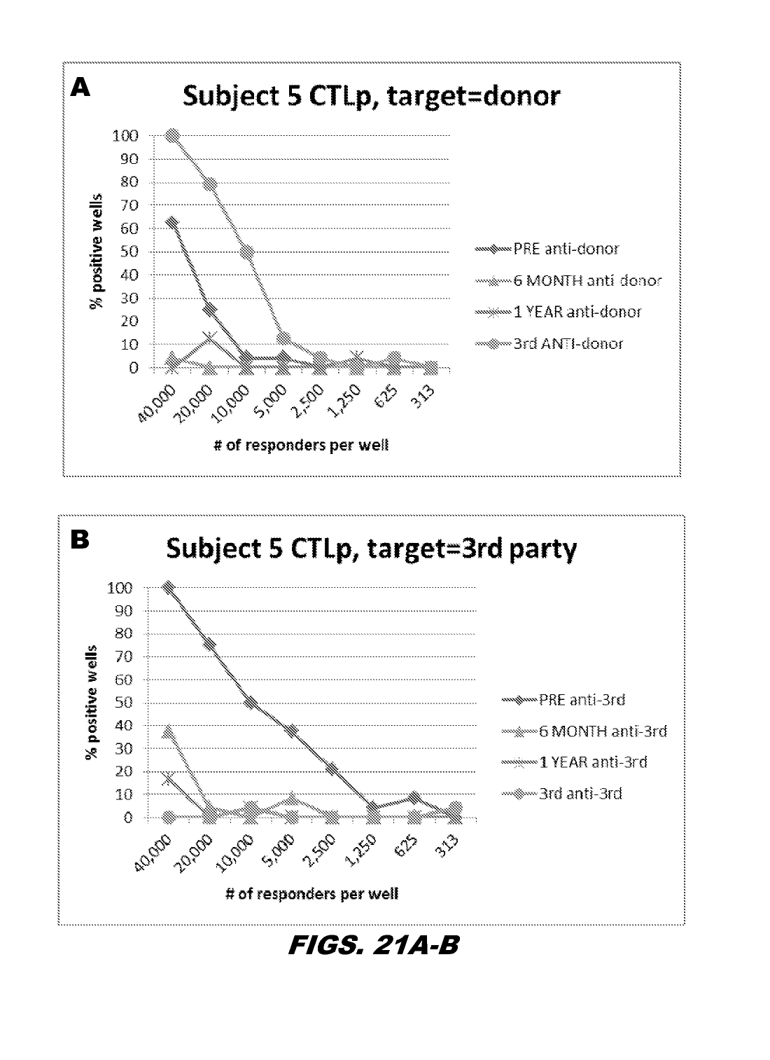

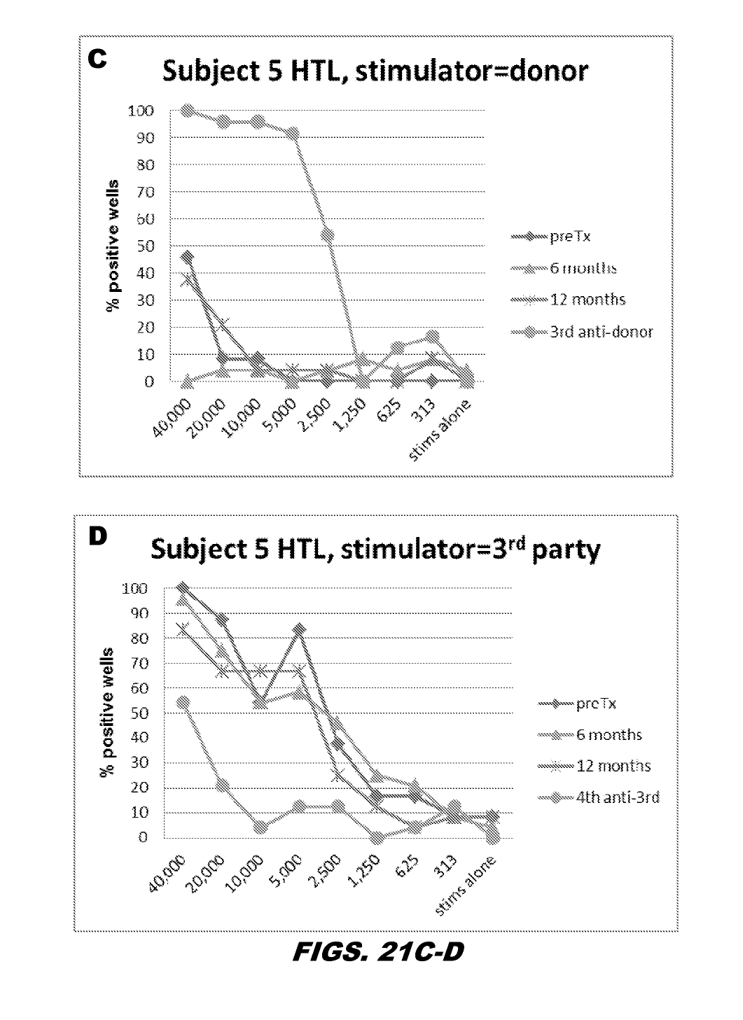

One month after discontinuation of immunosuppression 8 months post-CKBMT, Subject 5 developed acute graft rejection that culminated in graft loss despite anti-rejection therapy. Of the four CKBMT recipients studied, Subject 5 was the only one in which donor-reactive CD4 and CD8 clones showed no significant reduction after transplantation (FIGS. 9A-B and FIGS. 16A-F). Remarkably, the corresponding MLR and CML in this subject displayed a profile consistent with donor-specific tolerance, with complete loss of the anti-donor response (no greater than the anti-self, negative control response) at six months and one year, while a robust 3^{rd}-party MLR response (and weak 3^{rd} party CML response at 6 mohts) had recovered (FIGS. 10D and 11D). CTLp revealed early and persistent disappearance of measurable anti-donor CTL reactivity, whereas HTLs showed persistent anti-donor reactivity at the highest responder concentrations (FIGS. 21A-D).

In this non-tolerant CKBMT patient, the MLR, CML, and CTLp assays failed to reflect the presence of donor-reactive clones detected by clonal analysis and once again, the HTL was the most sensitive functional assay for anti-donor responsiveness. The functional assays did not distinguish the lack of tolerance that was observed clinically, whereas the clonal analysis showed a pattern (lack of deletion of donor-reactive clones) that was distinct from the three tolerant CKBMT patients.

Lack of Evidence for Deletion of Donor-Reactive Clones in Conventional Kidney Transplant Recipients

As a control to determine whether the deletion of donor-reactive TCR observed in CKBMT patients was related to their tolerant state, similar analyses were performed on two conventional kidney transplant recipients treated with standard immunosuppressive regimens. Subject IS#1 had end-stage renal disease (ESRD) secondary to focal segmental glomerulosclerosis (FSGS) and received a renal transplant from a living unrelated donor. She received thymoglobulin and methylprednisolone for induction therapy and was subsequently maintained on tacrolimus and mycophenolate. Allograft biopsies performed at 10 and 17 months after transplantation to evaluate acute rises in serum creatinine showed no evidence of cellular or antibody-mediated rejection, and were consistent with calcineurin inhibitor toxicity. CFSE MLR cultures were set-up as described above to isolate the pre-transplant donor-reactive repertoire and then searched for the presence of these clones in unstimulated PBMC samples obtained 10 and 17 months post-transplant. In contrast to the tolerant patients, the CD4+ donor-reactive clones were significantly enriched in the peripheral blood at both post-transplant timepointsrelative to pre-transplant and the donor-reactive CD8+ clone frequencies were not significantly changed (FIGS. 9A-B).

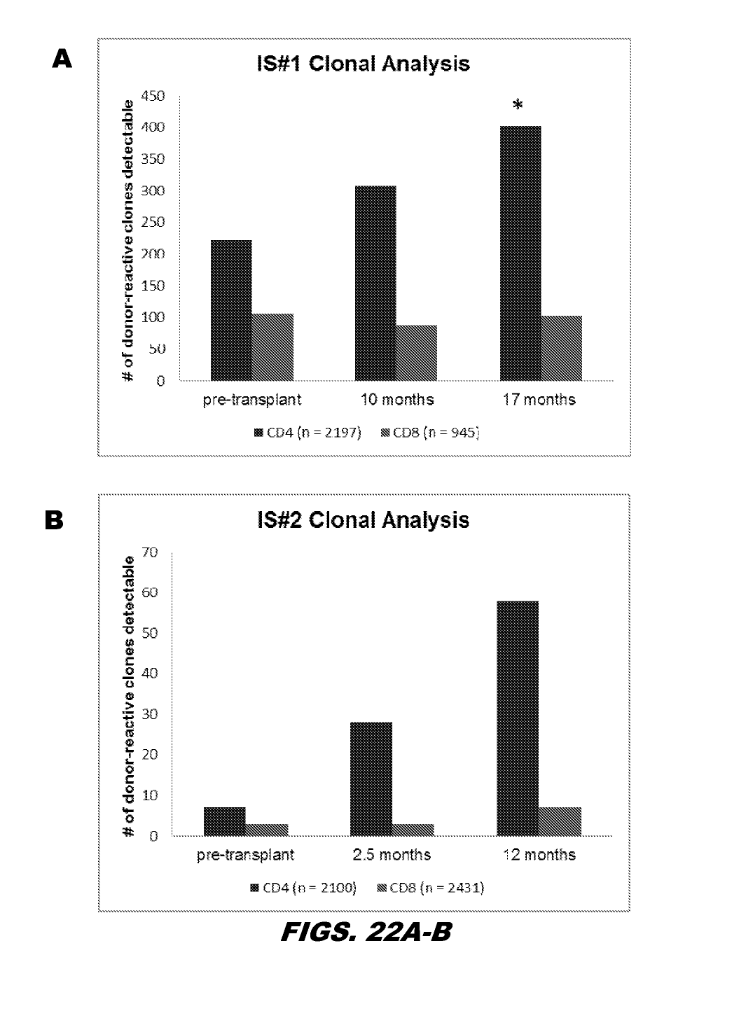

Subject IS#2 also had ESRD due to FSGS, and received a renal transplant from a living unrelated donor several years after a prior living-related transplant had failed. Because the patient was highly sensitized, he received plasmapheresis and IVIg preoperatively and received rituximab, basiliximab, and methylprednisolone as induction therapy. A three-month protocol biopsy revealed Banff grade 1B acute cellular rejection (ACR), which was treated with thymoglobulin and corticosteroids. Subsequent protocol biopsies at 6 months and 1 year were suspicious for ongoing rejection. PBMC from IS#2 at 10 weeks and one year post-transplant were examined for the donor-reactive clones identified pre-transplant. The numbers of detectable donor-reactive CD4+ clones were significantly increased at both time points. Donor-reactive CD8 clones did change significantly (FIGS. 9A-B, FIGS. 16A-F, FIGS. 22A-B). Thus, in both conventional kidney transplant patients, significant enrichment of donor-reactive CD4 clones, but not CD8 clones, was observed after transplantation.

The P values resulting from comparison of post-transplant to pre-transplant numbers of detectable donor-reactive clones in all six subjects are summarized in FIG. 9C. Subjects 1, 2, and 4 are referred to as “tolerant” because they have all achieved excellent allograft function for years in the absence of immunosuppression. Subject 5, who rejected his allograft at 9 months post-CKBMT, and both kidney transplant recipients on standard immunosuppressive regimens (IS#1 and IS#2) are classified as non-tolerant. Almost all samples from the tolerant subjects exhibit reductions in CD4+ and CD8+ donor-reactive clones after transplant, and these reductions frequently achieved statistical significance (dots falling above horizontal dotted line). The samples from the non-tolerant patients, on the other hand, almost all exhibit increases in donor-reactive clones after transplant, with expansions in CD4+ clones reaching statistical significance. Thus, in these six subjects, tracking of donor-reactive clones distinguishes between tolerant and non-tolerant states.

FIGS. 16 A-F shows that the degree of expansion of donor-reactive CD4 cells increased as the criterion defining donor reactivity was increased to require greater degrees of expansion in pre-transplant MLR for all 3 non-tolerant patients, especially at later time points, suggesting that the clones responding most strongly to the donor in pre-transplant MLR were those most likely to persist and expand post-transplant in a non-tolerant patient.

T Cell Repertoire Turnover

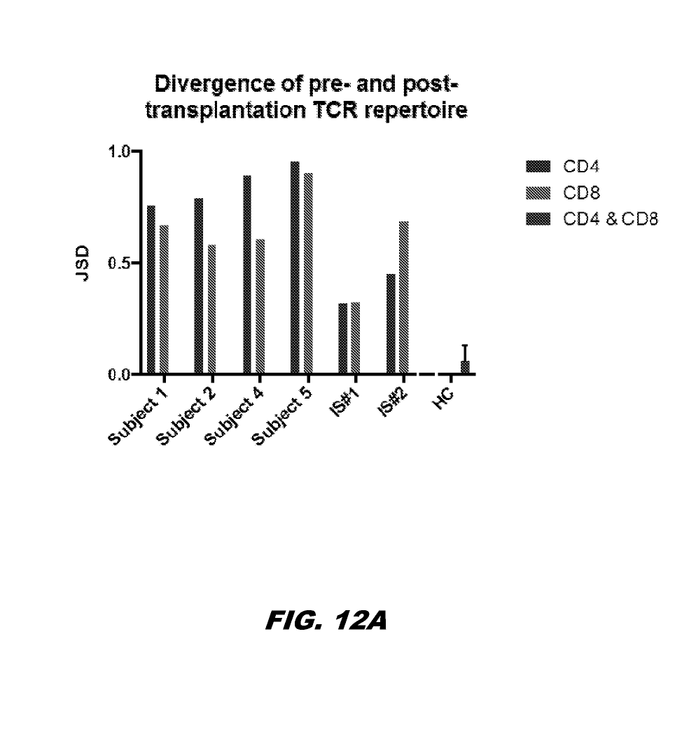

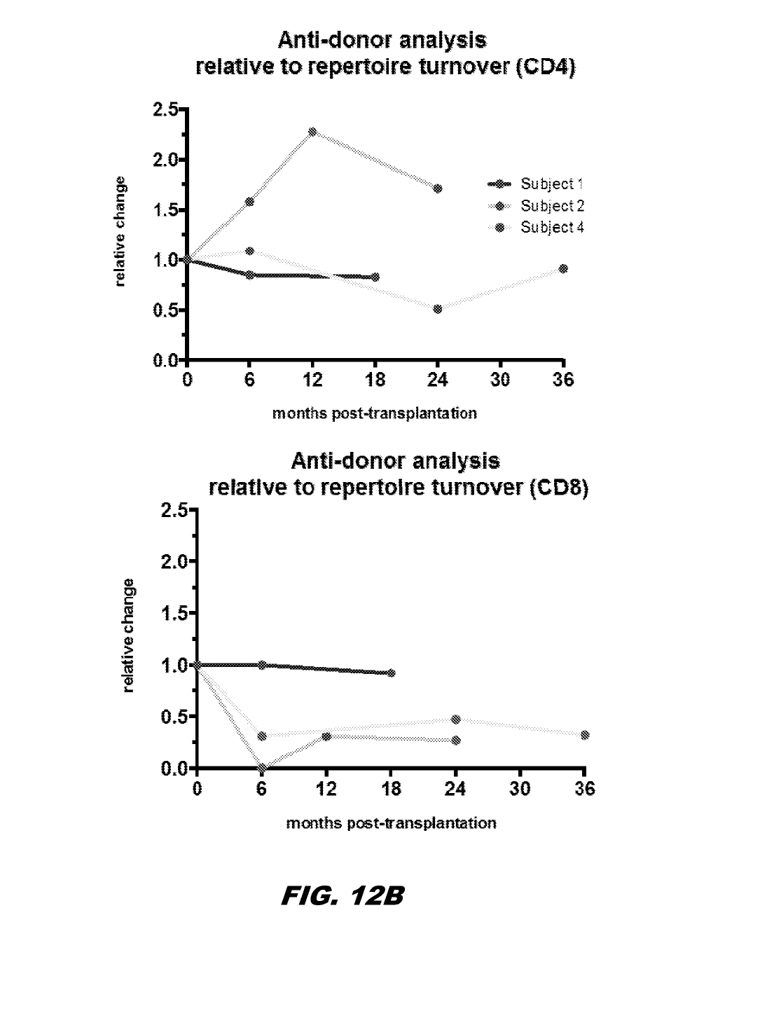

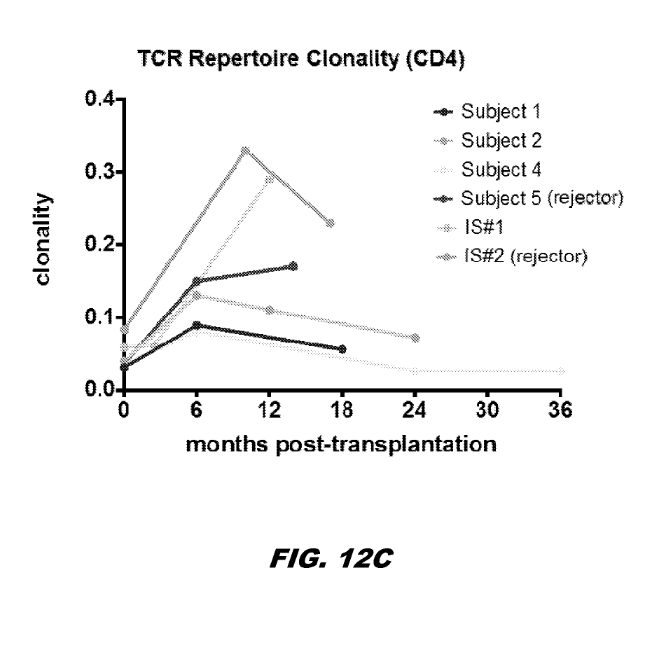

Without being bound by theory, one possible explanation for the loss of donor-specific clones in tolerant patients could be the global T cell depleting treatment that was included in the conditioning regimen, which could result in considerable repertoire turnover as T cells developed de novo from thymic recovery. To quantify the change in T cell repertoire over time, the Jensen-Shannon Divergence (JSD) was calculated on the top 1000 nucleotide sequences for pairs of pre- and post-transplant time points (FIG. 12A). The JSD measures divergence of the repertoire between two samples taking the clonal frequencies into account; a JSD of “1” reflects complete divergence of the two repertoires while the JSD of two repertoires with the same repertoire is “0”. For reference, the JSD of pairs of samples of T cells isolated from peripheral blood one year apart from four healthy controls was determined. All transplant patients, including IS#1 and IS#2, showed greater repertoire divergence compared to the healthy controls. However, the JSD values were higher for the CD4+ populations of the 4 CKBMT recipients than for the 2 conventional transplant recipients in the CD8+ T cell compartment. IS#2 showed similar repertoire turnover as the 4 CKBMT patients while that of IS#1 was lower. The non-tolerant CKBMT patient showed the highest JSD values of all, with almost complete turnover of the CD4 and CD8 repertoires (FIG. 12A).

In view of the high level of repertoire turnover in the CKBMT patients, it is possibile that post-transplant deletion of donor-reactive clones largely reflects the global depletion of pre-existing T cells and their replacement by new ones through de novo T cell development. To distinguish these possibilities, the likelihood of detecting clones post-transplant that were defined as either donor-reactive or non-donor-reactive in the pre-transplant MLR was compared. Among CD4+ T cells, no significant decrease in donor-reactive compared to non-donor-reactive clones was observed post-transplant, and Subject 2 even showed a significant relative increase in donor-reactive compared to non-donor-reactive CD4 cells at 1 year (FIG. 12B). A significant and sustained reduction in the detection of donor-reactive compared to non-donor-reactive CD8+ T cells was observed in Subjects 2 and 4, suggesting that the loss of donor-reactive CD8 clones was antigen-driven (FIG. 12B).

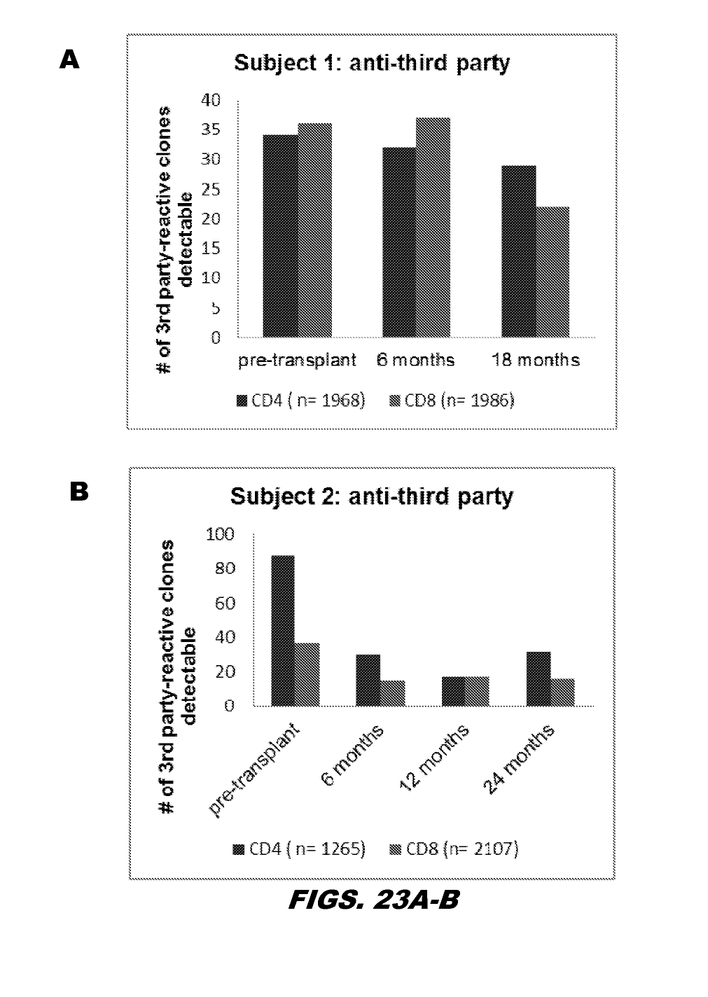

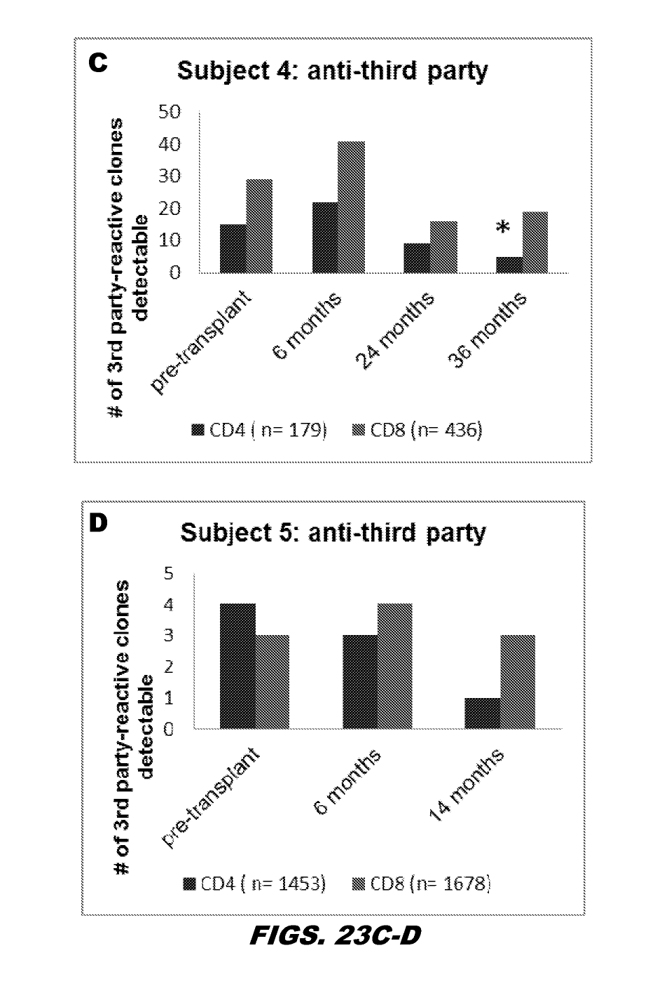

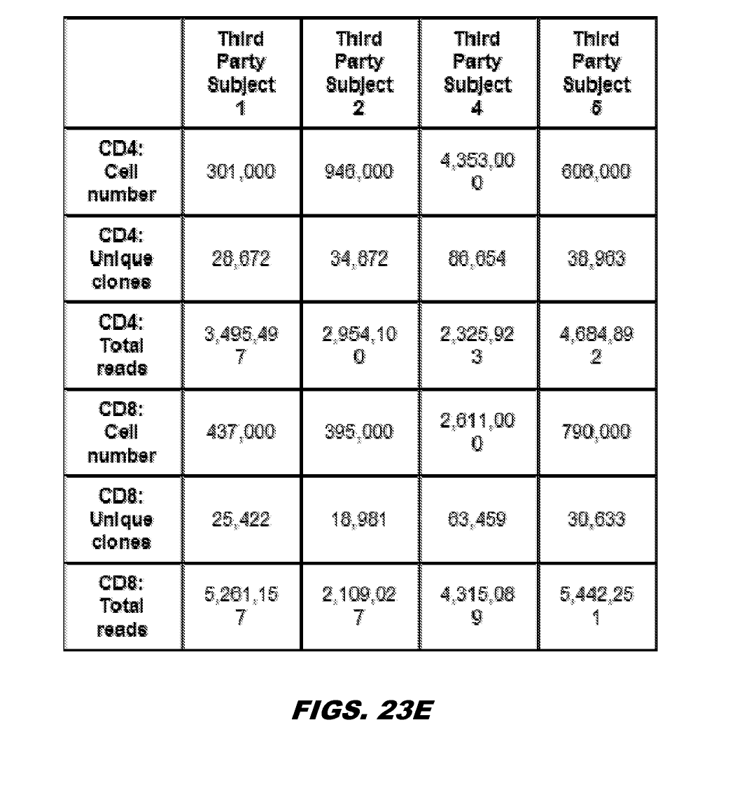

Some, but not all deletion of donor-reactive clones could be donor antigen-driven in tolerant CKBMT patients and was supported by analysis of the fate of third party-reactive T cells identified in pre-transplant MLRs using third party stimulators (FIGS. 23A-E). In Subjects 1 and 4, there was no statistically significant decrease in anti-third party clones, with the exception of Subject 1 CD8 cells at 18 months. In contrast, anti-third party CD4 and CD8 clones declined significantly at all time points in Subject 2. This loss of third party-reactive T cells was not due to overlap between the anti-donor and anti-third party repertoires.

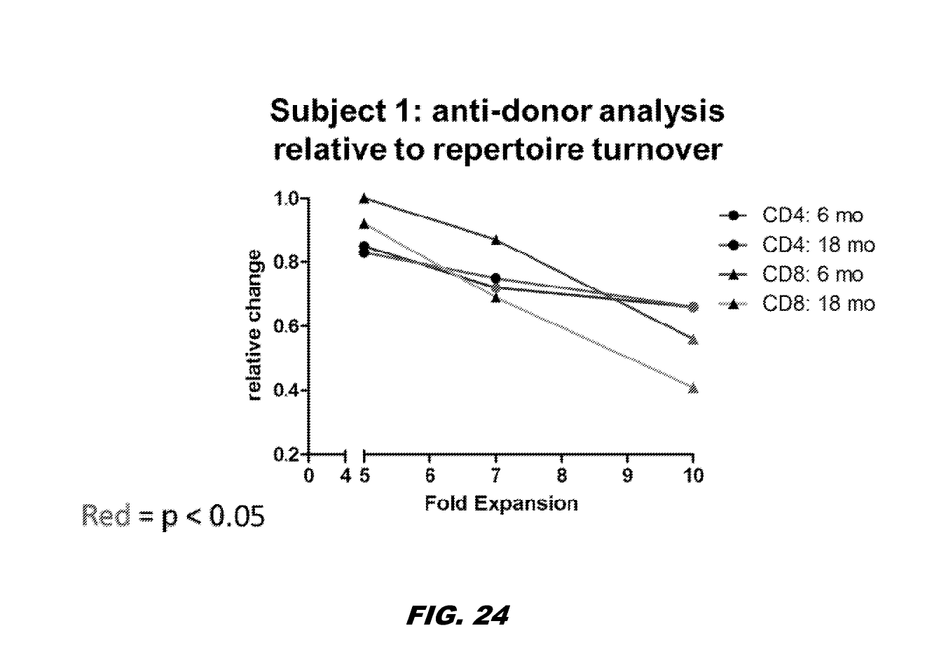

The ability to detect a relative loss of donor-reactive compared to non-donor-reactive clones varied considerably with the definition of “donor-reactive”. For example, as shown in FIG. 24 for Subject 1, when this definition required an increasing degree of expansion compared to the 5-fold definition used throughout this discussion, an increasing degree of relative loss of donor-reactive clones was seen, so that CD4 and CD8 deletion of clones that expanded 10-fold in pre-transplant anti-donor MLR was highly significant at both post-transplant time points. These data suggest that the most strongly donor-reactive clones were more likely to show deletion following CKBMT. Overall, the post-transplant decline in donor-reactive T cells in tolerant patients therefore may reflect a mixture of repertoire turnover and specific deletion of donor-reactive T cells, combined with some degree of antigen-driven expansion.

Reduced TCR Diversity in Non-Tolerant Compared to Tolerant Subjects

Since human T cells undergo lymphopenia-driven expansion in a T cell-deficient environment (12) and this may reduce the repertoire diversity associated with T cell reconstitution after lymphablative conditioning (13) the overall clonality of the T cell populations post-transplant was compared to assess repertoire diversity (FIG. 12C). A T cell repertoire composed entirely of identical clones has a clonality of “1” while that of a T cell pool of all unique clones is “0.” In contrast to the 3 tolerant subjects, in whom the T cell clonality among CD4+ T cells returned to pre-transplant values, the 3 non-tolerant patients showed increased clonality following transplantation, consistent with grast-driven selection of donor-specific clones in these non-tolerant patients (FIG. 12C). No difference was seen between the tolerant and non-tolerant patients in the clonality of the CD8 repertoire.

Discussion

T cell responses to allogeneic MHC molecules are orders of magnitude stronger than any other type of immune response (8, 9, 14, 15), presumably reflecting recognition by a large repertoire of T cell receptors, thus far precluding the ability to identify and track donor-specific T cells following transplantation. Described herein is the development of a novel approach using high throughput deep TCR sequencing technology to identify, prior to transplant, and track, following the transplant, human transplant recipients' repertoires of donor-reactive T cells. These studies have demonstrated the feasibility of this approach and suggested its superiority over functional assays in identifying a tolerant state. In addition, they provide evidence for clonal deletion as a major mechanism of tolerance following CKBMT.

In the NKD03 trial, MLR, CML and LDA studies revealed lasting donor-specific unresponsiveness in long-term tolerant CKBMT patients. The functional and phenotypic data suggested that regulatory mechanisms might play a role in initial tolerance, and that clonal deletion might contribute to longer-term tolerance (1, 7). These studies, however, could not distinguish between long-term anergy and deletion of donor-reactive T cells. The new clonal analysis approach described herein has provided new insight into the mechanisms of tolerance in CKBMT patients. Donor-reactive CD4+ and CD8+ clones, identified prior to transplantation by CFSE-MLR, decreased in the circulating pool after transplantation in three of three tolerant CKBMT subjects. The data are consistent with deletion of donor-reactive clones was either partial or not apparent at the earliest time point (6 months) and, especially for CD4+ cells, evolved over time in patients who achieved long-term tolerance. In the only CKBMT subject who failed to achieve operational tolerance, a reduction of donor-specific clones did not occur. Thus, for the first time direct evidence has been obtained for a role for deletion in the maintenance of allograft tolerance in humans. It can be possible that donor-reactive clones may move from the circulation to the allograft, although this is unlikely to explain the loss of these clones from the circulation, as serial protocol biopsies have shown an absence of rejection in association with minimal mononuclear cell infiltrates, which are enriched for Foxp3+ cells, in the tolerant CKBMT recipients (1, 2).

Some, but not all of the deletion of donor-reactive clones in blood of tolerant patients may be explained by global T cell depletion induced by conditioning, as the data show considerable TCR repertoire turnover following transplant. Although this global depletion is marked (1, 2, 6, 7), residual donor-reactive cells may be specifically expanded in the post-transplant environment. The initial recovery of T cells is most likely driven by lymphopenia-induced proliferation (LIP), as the vast majority of cells express an effector/memory phenotype in the first 3-6 months post-transplant (6). Given that fast LIP has been shown to occur and result in effector/memory phenotypic conversion for human T cells (12), and that fast LIP is largely antigen-driven (16, 17), residual donor-reactive clones are likely to be selectively expanded in the periphery of T cell-depleted transplant recipients by the presence of the bone marrow and/or kidney allograft as a source of antigen.

Consistent with the notion of antigen-driven expansion of donor-reactive clones in vivo, two of two conventional transplant recipients showed significant and persistent expansion of these clones following transplant, despite the fact that one such patient also received T cell-depleting induction therapy and had considerable repertoire turnover following the transplant. Of note, the degree of expansion of donor-reactive CD4 cells increased as the criterion defining donor reactivity required greater degrees of expansion in pre-transplant MLR for all 3 non-tolerant patients (FIGS. 16 A-F), suggesting that the clones responding most strongly to the donor in pre-transplant MLR were those most likely to persist and expand. These results also suggest that the pre-transplant MLR identifies donor-reactive clones that are highly relevant to transplantation and are the first data to establish a clear role for MLR-defined alloreactive T cells in HLA-mismatched human transplant recipients. Previous studies have identified a CD4 clone recognizing recipient minor histocompatibility antigens in MLR as having a role in GVHD in an HLA-identical hematopoietic cell transplant recipient (18), but this study is the first to examine the entire repertoire of alloreactive cells and to do so in an HLA-mismatched setting.

Given the similar, high level of repertoire turnover in the tolerant and non-tolerant CKBMT recipients and the fact that absolute reduction of donor-reactive clones was seen only in the tolerant and not the non-tolerant recipient, it is difficult to attribute the donor-specific deletion only to global T cell depletion and repertoire turnover. The persistence of some pre-existing donor-reactive clones, along with the progressive deletion observed over time in several tolerant patients suggest that some degree of antigen-driven expansion of the few persisting donor-reactive clones also occurs in tolerant subjects during the early, lymphopenic period. Indeed, one tolerant subject, while showing an absolute loss of donor-reactive CD4 clones (FIGS. 9A-C), showed an increase in these clones relative to non-donor-reactive clones, which are mostly not under antigenic proliferative pressure, at the 6 month time point (FIG. 12B). Thus, the relative numbers of donor-reactive and non-donor-reactive clones must be regarded as the net effect of T cell-depleting conditioning (for all clones), antigen-driven expansion (donor-reactive clones) and antigen-driven deletion (donor-reactive clones). In the case of rejection, it also seems likely that entry of donor-reactive clones into the graft influences clonal frequencies in the circulation. In Subject 5, who failed to achieve tolerance, the net effect of these processes was no change in the absolute rate of detection of donor-reactive clones post-compared to pre-transplant, whereas the tolerant patients showed a significant decline. Despite the high level of overall repertoire turnover and the evidence for initial expansion of donor-reactive clones in tolerant patients, the loss of donor-reactive T cell clones was greater than that of non-donor-reactive and/or of third party-reactive clones in a number of instances in all 3 tolerant subjects. Thus, when tolerance is achieved, the data suggest that these expanded donor-reactive clones are gradually deleted, resulting in many cases in greater clonal reduction, especially among clones with strongest anti-donor reactivity, compared to globally reduced non-donor-reactive clones (FIG. 24).

The reduction in (but not complete disappearance of) donor-reactive clones in tolerant patients is consistent with our hypothesis that initial regulatory mechanisms give way to eventual deletion of donor-reactive T cells in response to repeated encounter with donor antigens on the quiescent, accepted kidney allograft (7). This late clonal deletion most likely occurs in the periphery rather than the thymus, as hematopoietic chimerism was short-lived in these patients (6), making intrathymic deletion by donor APCs unlikely in the long-term. Nevertheless, peripheral APCs picking up donor antigen in intact or processed form might migrate to the thymus as described (19) and mediate ongoing central deletion as the de novo thymopoiesis recovers in 3-6 months (6). In tolerant CKBMT Subject 4, who was unique among tolerant CKBMT recipients in showing persistent anti-donor MLR and CML reactivity following the transplant, clonal analysis of the anti-donor MLR at 1 year indicated that this response was mediated by T cell clones that developed de novo post-transplant, consistent with failure to delete newly developing donor-reactive T cells in the thymus due to absence of persistent chimerism. Despite this development of new donor-reactive clones following the transplant, this patient has demonstrated operational tolerance for a period of years. Witout being bound by theory, it is possible that regulatory cells in the graft may play a role in maintaining this tolerance.

Clonal analysis was more sensitive in detecting persistent donor-reactive clones than were MLR, CML, and CTLp assays, which revealed donor-specific unresponsiveness in Subjects 1, 2, and 5. For all CKBMT patients, HTL LDA appeared to be the most sensitive of the functional assays for detecting persisting donor-reactive clones. Donor-specific unresponsiveness in the assays above was particularly surprising in Subject 5, given that he rejected his allograft. In this patient, clonal analysis was the only assay to demonstrate a distinct pattern of persistent donor reactivity after transplant, distinguishing him from the three tolerant CKBMT recipients. The enhanced sensitivity of clonal analysis is likely explained by the fact that, unlike the in vitro functional assays, it is not affected by anergy or suppression. The lack of predictive value of MLR and CML with respect to graft outcomes is consistent with prior studies in animals (20, 21), patients receiving conventional transplants (22-26) and patients receiving a different CKBMT protocol for HLA-mismatched kidney allograft tolerance induction (27). Overall, clonal analysis accurately distinguished between tolerance and non-tolerance in the cohort of six subjects. This approach of defining alloreactive clones pre-transplant and tracking them post-transplant provides a new and specific method of assessing transplantation tolerance.

The similar clonal behavior in two standard kidney transplant recipients despite disparate clinical outcomes (IS#1 had no rejection episodes, whereas IS#2 had ACR1b) may partly reflect the limited cell numbers available prior to and 1 year following transplant for IS#2, which resulted in a higher threshold frequency to declare a clone “present”. Moreover, important differences in donor-reactive T cell clone numbers might be present in the kidney graft and not the peripheral T cell pool. Analysis of allograft biopsy specimens in addition to the peripheral blood of a larger series of patients can be done. Additionally, the pre-transplant MLR is biased toward detecting directly alloreactive T-cell clones which likely contribute to chronic graft dysfunction in addition to driving acute rejection (28, 29). the approach described herein may not capture indirect anti-donor alloreactivity, which also plays an important role in allograft rejection (30-32).

Repertoire turnover between the pre- and post-transplant periods was greater for CKBMT patients compared to conventional transplant recipients, consistent with more exhaustive T cell depletion associated with conditioning for CKBMT. Both groups showed much greater TCR turnover than healthy controls over the same 1-year period, indicating that induction and immunosuppression for conventional transplantation also leads to significant repertoire turnover. Somewhat greater repertoire divergence was observed in the CKBMT patients for CD4+ cells than CD8+ T cells, consistent with a recent report (33) of increased CD8 repertoire persistence in patients with multiple sclerosis (MS) who received conditioning followed by autologous stem cell transplantation. Another central observation in the MS study was that T cell diversity recovered more quickly in patients who responded to treatment compared to non-responders. CD4 T cell diversity returned to baseline levels more rapidly in our 3 tolerant patients than in the 3 non-tolerant subjects (FIG. 12B). This return of diversity did not correlate with recovery of naïve-type CD4 cells, was more rapid in CKBMT Subjects 4 and 5 than in Subjects 1 and 2 (6). These results suggest that persistently reduced TCR diversity may reflect peripheral antigen-driven selection for expansion of alloreactive and/or autoreactive T cell clones in non-tolerant subjects, perhaps reflecting a failure of regulatory mechanisms.

In conclusion, described herein is a novel method whereby pre-existing donor-reactive recipient T cell clones may be tracked prospectively after transplantation. This method has been used to obtain evidence for a role for deletion of donor-reactive CD4 and CD8 T cells in the maintenance of tolerance in CKBMT patients with transient chimerism. A recent study (34) reported the use of high throughput CDR3 sequencing to analyze donor antigen-responsive T cells and showed the presence of alloantigen-specific T cell clones in the grafts and urine of a single patient with allograft dysfunction. However, that study relied on post-transplant MLR to identify donor-reactive clones, therefore depending on persistence of a measurable MLR following transplant and precluding information on the fate of pre-existing donor-reactive T cells. The approach described herein involves identification of donor-reactive clones prior to transplantation and then tracking them prospectively in order to study their fate over time. This avoids the dependence on functional assays post-transplant, which correlate poorly with the presence or absence of tolerance. Clonal analysis outperformed “conventional” in-vitro assays of tolerance (MLR, CML, and LDA) as a biomarker for tolerance or its failure in these patients. The ability of our new strategy to provide mechanistic insights into tolerance has been demonstrated. Future studies using blood and graft specimens from large cohorts of conventional transplant recipients and patients achieving tolerance with additional regimens will reveal its full potential for predicting graft outcomes and revealing mechanisms of rejection and tolerance.

Methods

Mixed Lymphocyte Reactions: Preparation of CFSE-labeled responders: For the pilot study (T1 and T2 stim samples) and ITN Subjects 1 and 2, MLRs were set up using purified T-cells as responders. Previously frozen pre-transplant PBMCs were thawed, washed, and resuspended in MACS buffer. MACS beads (Pan T cell Isolation Kit II, Miltenyi Biotec catalog #130-091-156) were used to generate “untouched” T cells. These T cells were resuspended in PBS at 1×106 cells/mL, labeled with CFSE at a concentration of 0.2 uM-0.5 uM (CellTrace CFSE Proliferation Kit, Molecular Probes™), washed 3 times, and resuspended in MLR medium (AIM-V supplemented with 5% AB heat-inactivated human serum, 0.01M HEPES, and 50 uM 2-ME at a concentration of 2×106 cells/mL. For CKBMT Subjects 4 and 5, the two conventional transplant recipients, and the anti-third party responses, whole PBMC were used as responders instead of purified T cells. PBMCs were labeled with CFSE as above and resuspended at 2×106 cells/mL. Preparation of violet dye-labeled stimulators: Cryopreserved donor PBMCs were thawed, washed, resuspended in PBS, and labeled with BD Horizon™ Violet Proliferation Dye 450. After labeling, cells were washed twice, resuspended in MLR medium at 2×106 cells/mL, and irradiated at 30-35 Gray. Plating of cells: One million CFSE-labeled pre-transplant responder cells and one million violet dye-labeled irradiated stimulators were plated in each well of a 24-well plate (total well volume 1 mL). For IS#1, IS#2, and the anti-third party MLRs, we used 96-well plates with each well containing 200,000 responder PBMC and 200,000 stimulators (total well volume 200 uL). MLR cultures were incubated at 37 C for 5-6 days.

Flow Cytometry: MLR wells were harvested after 6 days of culture. Cells were resuspended in FACS buffer, stained for 30 minutes with fluorochome-conjugated antibodies against CD3, CD4, and CD8, washed and filtered before FACS sorting on a BDTM LSR II Flow Cytometer to isolate two discrete cell populations (violet-CD3+CD4+CFSElo and violet-CD3+CD8+CFSElo) representing the CD4+ and CD8+ recipient-derived donor-reactive T cells. For unstimulated cell populations, PBMC were thawed and stained with anti-CD3, CD4, and CD8, then FACS sorted into CD3+CD4+ and CD3+CD8+ populations.

DNA isolation and sequencing: Genomic DNA was isolated from sorted cell populations using the Qiagen DNeasy Blood and Tissue Kit. DNA was frozen at −20 C and shipped on dry ice to Adaptive Biotechnologies (Seattle, Wash.) for high-throughput CDR3 sequencing. The nucleotide sequence data were retrieved from Adaptive's ImmunoSeq software.

In-Vitro Immunologic Assays: Standard MLR, CML, and LDA assays were performed using the methods detailed previously (35).

Computational and statistical analysis: Mapping of the reads, identification of CDR3 regions and V/J genes, and bias adjustment were performed by Adaptive (36) through their proprietary software. We receive tabulated TRB sequencing data from Adaptive, including CDR3 nucleotide and amino acid sequences, raw copy number (read counts), adjusted copy number and frequency, V/J genes and gene families, inferred insertions and deletions in V-D-J junctions etc.

Diversity of a repertoire: The diversity of each repertoire was measured by entropy (37) (H≡Σpᵢ log₂ pᵢ, where pᵢ is the frequency of clone i) and clonality (S≡1−H_{obs}/Hₘₐₓ), where Hₘₐₓ is the entropy of a repertoire with the same number of clones, each having exactly the same frequency.

Comparison of repertoires. The difference between two repertoires was measured using Jensen-Shannon divergence (38) and Pearson correlation, both of which ranges from 0 to 1. Explanded clones were defined in MLR by a minimum frequency in stimulated samples (f; f is set at 0.01%) and fold change (C=frequency in stimulated pre-transplant samples/frequency in unstimulated pre-transplant samples; C is conventionally set at 5). A clone is defined as detectable if the frequency is larger than a threshold (m; m is usually 0.001% for samples with ≧106 T cells, when an individual cell has a 90% chance of being detected if the TCR profiling efficiency is ˜20%, a lower bound of the Immunoseq technology).

Hypothesis testing. To test expansion or deletion of clones, donor-reactive unique clones were first identified (defined as above) in pre-transplant MLR, counted their number (N), and tested if these clones are equally likely to be detectable in unstimulated pre- and post-transplant samples. Specifically, we generate a 2×2 contingence table: [Dₚᵣₑ, N−Dₚᵣₑ; Dₚₒₛₜ, N−Dₚₒₛₜ], where Dₚᵣₑ is the number of detectable pre-identified donor- (or third party-) reactive clones in unstimulated pre-transplant and Dₚₒₛₜ is the number of detectable clones in unstimulated post-transplant samples. Two-sided Fisher's exact test is performed and p-values and odds ratios reported.

### REFERENCES

1. Kawai T, Cosimi A B, Spitzer T R, Tolkoff-Rubin N, Suthanthiran M, Saidman S L, et al. HLA-mismatched renal transplantation without maintenance immunosuppression. N Engl J Med. 2008 Jan. 24; 358(4):353-61. PubMed PMID: 18216355. Pubmed Central PMCID: 2819046. Epub 2008/01/25. eng.
2. Kawai T, Sachs D H, Sykes M, Cosimi A B, Immune Tolerance N. HLA-mismatched renal transplantation without maintenance immunosuppression. The New England journal of medicine. 2013 May 9; 368(19):1850-2. PubMed PMID: 23656665.
3. Manilay J O, Pearson D A, Sergio J J, Swenson K G, Sykes M. Intrathymic deletion of alloreactive T cells in mixed bone marrow chimeras prepared with a nonmyeloablative conditioning regimen. Transplantation. 1998 Jul. 15; 66(1):96-102. PubMed PMID: 9679828. Epub 1998/07/29. eng.
4. Sharabi Y, Sachs D H. Mixed chimerism and permanent specific transplantation tolerance induced by a nonlethal preparative regimen. The Journal of experimental medicine. 1989 Feb. 1; 169(2):493-502. PubMed PMID: 2562984. Pubmed Central PMCID: PMC2189213. Epub 1989/02/01. eng.
5. Tomita Y, Khan A, Sykes M. Role of intrathymic clonal deletion and peripheral anergy in transplantation tolerance induced by bone marrow transplantation in mice conditioned with a nonmyeloablative regimen. Journal of immunology (Baltimore, Md.: 1950). 1994 Aug. 1; 153(3):1087-98. PubMed PMID: 8027542. Epub 1994/08/01. eng.
6. LoCascio S A, Morokata T, Chittenden M, Preffer F I, Dombkowski D M, Andreola G, et al. Mixed chimerism, lymphocyte recovery, and evidence for early donor-specific unresponsiveness in patients receiving combined kidney and bone marrow transplantation to induce tolerance. Transplantation. 2010 Dec. 27; 90(12):1607-15. PubMed PMID: 21085064. Pubmed Central PMCID: 3059844. Epub 2010/11/19. eng.
7. Andreola G, Chittenden M, Shaffer J, Cosimi A B, Kawai T, Cotter P, et al. Mechanisms of donor-specific tolerance in recipients of haploidentical combined bone marrow/kidney transplantation. American journal of transplantation: official journal of the American Society of Transplantation and the American Society of Transplant Surgeons. 2011 June; 11(6):1236-47. PubMed PMID: 21645255. Pubmed Central PMCID: 3140222. Epub 2011/06/08. eng.
8. Sherman L A, Chattopadhyay S. The molecular basis of allorecognition. Annu Rev Immunol. 1993; 11:385-402. PubMed PMID: 8476567. Epub 1993/01/01. eng.
9. Suchin E J, Langmuir P B, Palmer E, Sayegh M H, Wells A D, Turka L A. Quantifying the frequency of alloreactive T cells in vivo: new answers to an old question. Journal of immunology (Baltimore, Md.: 1950). 2001 Jan. 15; 166(2):973-81. PubMed PMID: 11145675. Epub 2001/01/06. eng.
10. Robins H S, Campregher P V, Srivastava S K, Wacher A, Turtle C J, Kahsai O, et al. Comprehensive assessment of T-cell receptor beta-chain diversity in alphabeta T cells. Blood. 2009 Nov. 5; 114(19):4099-107. PubMed PMID: 19706884. Pubmed Central PMCID: 2774550. Epub 2009/08/27. eng.
11. Robins H, Desmarais C, Matthis J, Livingston R, Andriesen J, Reijonen H, et al. Ultra-sensitive detection of rare T cell clones. J Immunol Methods. 2012 Jan. 31; 375(1-2):14-9. PubMed PMID: 21945395. Pubmed Central PMCID: 3721519. Epub 2011/09/29. eng.
12. Onoe T, Kalscheuer H, Chittenden M, Zhao G, Yang Y-G, Sykes M. Homeostatic expansion and phenotypic conversion of human T cells depend on peripheral interactions with APCs. Journal of immunology. 2010 6/15/2010; 184(12):6756-65.
13. Rosenblum J M, Kirk A D. Recollective homeostasis and the immune consequences of peritransplant depletional induction therapy. Immunological reviews. 2014 March; 258(1):167-82. PubMed PMID: 24517433. Epub 2014/02/13. eng.
14. Guillet M, Sebille F, Soulillou J. TCR usage in naive and committed alloreactive cells: implications for the understanding of TCR biases in transplantation. Current opinion in immunology. 2001 October; 13(5):566-71. PubMed PMID: 11544005. Epub 2001/09/07. eng.
15. Smith C, Miles J J, Khanna R. Advances in direct T-cell alloreactivity: function, avidity, biophysics and structure. American journal of transplantation: official journal of the American Society of Transplantation and the American Society of Transplant Surgeons. 2012 January; 12(1):15-26. PubMed PMID: 22152064. Epub 2011/12/14. eng.
16. Min B, Yamane H, Hu-Li J, Paul W E. Spontaneous and homeostatic proliferation of CD4 T cells are regulated by different mechanisms. Journal of immunology. 2005 5/15/2005; 174(10):6039-44.
17. Surh C D, Sprent J. Homeostasis of naive and memory T cells. Immunity. 2008 12/2008; 29(6):848-62.
18. Michalek J, Collins R H, Hill B J, Brenchley J M, Douek D C. Identification and monitoring of graft-versus-host specific T-cell clone in stem cell transplantation. Lancet. 2003 4/5/2003; 361(9364): 1183-5.
19. Bonasio R, Scimone M L, Schaerli P, Grabie N, Lichtman A H, von Andrian U H. Clonal deletion of thymocytes by circulating dendritic cells homing to the thymus. Nature immunology. 2006 10/2006; 7(10):1092-100.
20. Streilein J W, Strome P, Wood P J. Failure of in vitro assays to predict accurately the existence of neonatally induced H-2 tolerance. Transplantation. 1989 October; 48(4):630-4. PubMed PMID: 2529680. Epub 1989/10/01. eng.
21. Murase N, Kim D G, Todo S, Cramer D V, Fung J, Starzl T E. FK506 suppression of heart and liver allograft rejection. II: The induction of graft acceptance in rats. Transplantation. 1990 November; 50(5):739-44. PubMed PMID: 1700504. Pubmed Central PMCID: 2956599. Epub 1990/11/01. eng.
22. Cullen P R, Lester S, Rouch J, Morris P J. Mixed lymphocyte reaction and graft survival in forty cadaveric renal transplants. Clin Exp Immunol. 1977 May; 28(2):218-22. PubMed PMID: 141350. Pubmed Central PMCID: 1540754. Epub 1977/05/01. eng.
23. Goulmy E, Persijn G, Blokland E, D'Amaro J, van Rood J J. Cell-mediated lympholysis studies in renal allograft recipients. Transplantation. 1981 March; 31(3):210-7. PubMed PMID: 7015616. Epub 1981/03/01. eng.
24. Goulmy E, Stijnen T, Groenewoud A F, Persijn G G, Blokland E, Pool J, et al. Renal transplant patients monitored by the cell-mediated lympholysis assay. Evaluation of its clinical value. Transplantation. 1989 October; 48(4):559-63. PubMed PMID: 2799908. Epub 1989/10/01. eng.
25. Kahn D, du Toit E, Jacobson J E, Creemers P. Induction of donor-specific tolerance or sensitization as measured by sequential MLC reactivity up to 24 months after renal transplantation. Transpl Int. 1994; 7 Suppl 1:S287-9. PubMed PMID: 11271227. Epub 1994/01/01. eng.
26. Najafian N, Albin M J, Newell K A. How can we measure immunologic tolerance in humans? J Am Soc Nephrol. 2006 October; 17(10):2652-63. PubMed PMID: 16928808. Epub 2006/08/25. eng.
27. Leventhal J, Abecassis M, Miller J, Gallon L, Tollerud D, Elliott M J, et al. Tolerance induction in HLA disparate living donor kidney transplantation by donor stem cell infusion: durable chimerism predicts outcome. Transplantation. 2013 Jan. 15; 95(1):169-76. PubMed PMID: 23222893. Pubmed Central PMCID: 3531567. Epub 2012/12/12. eng.
28. Bestard O, Nickel P, Cruzado J M, Schoenemann C, Boenisch O, Sefrin A, et al. Circulating alloreactive T cells correlate with graft function in longstanding renal transplant recipients. Journal of the American Society of Nephrology: JASN. 2008 July; 19(7):1419-29. PubMed PMID: 18417724. Pubmed Central PMCID: 2440291.
29. Nadazdin O, Boskovic S, Wee S L, Sogawa H, Koyama I, Colvin R B, et al. Contributions of direct and indirect alloresponses to chronic rejection of kidney allografts in nonhuman primates. Journal of immunology (Baltimore, Md.: 1950). 2011 Nov. 1; 187(9):4589-97. PubMed PMID: 21957140. Pubmed Central PMCID: 3197941.
30. Ali J M, Bolton E M, Bradley J A, Pettigrew G J. Allorecognition pathways in transplant rejection and tolerance. Transplantation. 2013 October 27; 96(8):681-8. PubMed PMID: 23715047.
31. Baker R J, Hernandez-Fuentes M P, Brookes P A, Chaudhry A N, Cook H T, Lechler R I. Loss of direct and maintenance of indirect alloresponses in renal allograft recipients: implications for the pathogenesis of chronic allograft nephropathy. Journal of immunology (Baltimore, Md.: 1950). 2001 Dec. 15; 167(12):7199-206. PubMed PMID: 11739543.
32. Gokmen M R, Lombardi G, Lechler R I. The importance of the indirect pathway of allorecognition in clinical transplantation. Current opinion in immunology. 2008 October; 20(5):568-74. PubMed PMID: 18655831.
33. Muraro P A, Robins H, Malhotra S, Howell M, Phippard D, Desmarais C, et al. T cell repertoire following autologous stem cell transplantation for multiple sclerosis. The Journal of clinical investigation. 2014 Mar. 3; 124(3):1168-72. PubMed PMID: 24531550. Pubmed Central PMCID: PMC3934160. Epub 2014/02/18. eng.
34. Dziubianau M, Hecht J, Kuchenbecker L, Sattler A, Stervbo U, Rodelsperger C, et al. TCR repertoire analysis by next generation sequencing allows complex differential diagnosis of T cell-related pathology. American journal of transplantation: official journal of the American Society of Transplantation and the American Society of Transplant Surgeons. 2013 November; 13(11):2842-54. PubMed PMID: 24020931.
35. Fudaba Y, Spitzer T R, Shaffer J, Kawai T, Fehr T, Delmonico F, et al. Myeloma responses and tolerance following combined kidney and nonmyeloablative marrow transplantation: in vivo and in vitro analyses. American journal of transplantation: official journal of the American Society of Transplantation and the American Society of Transplant Surgeons. 2006 September; 6(9):2121-33. PubMed PMID: 16796719. Epub 2006/06/27. eng.
36. Robins H S, Campregher P V, Srivastava S K, Wacher A, Turtle C J, Kahsai O, et al. Comprehensive assessment of T-cell receptor beta-chain diversity in alphabeta T cells. Blood. 2009 11/5/2009; 114(19):4099-107.
37. Hill M O. Diversity and Evenness: A Unifying Notation and Its Consequences. Ecology. 1973 1973/03/01; 54(2):427-32.
38. Manning C D, Schütze H. Foundations of statistical natural language processing. Cambridge, Mass.: MIT Press; 1999. xxxvii, 680 p. p.

### Example 4

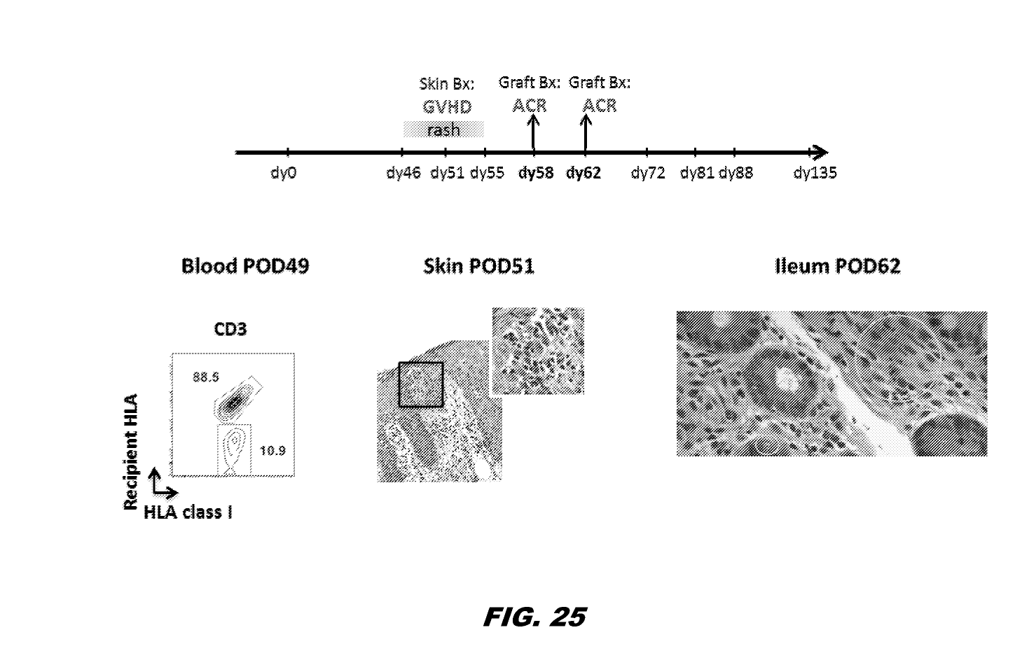

FIG. 25 shows alloreactice clones tracking in one intestinal transplant patient who experienced skin graft versus host disease (GVHD) and subsequently intestinal transplant rejection.

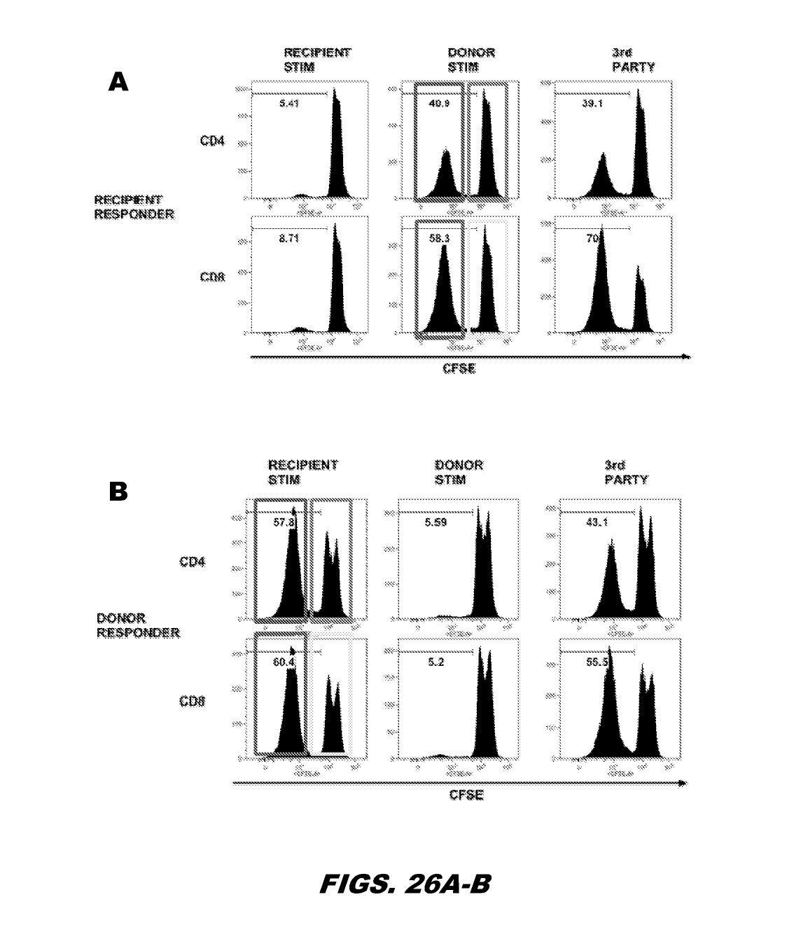

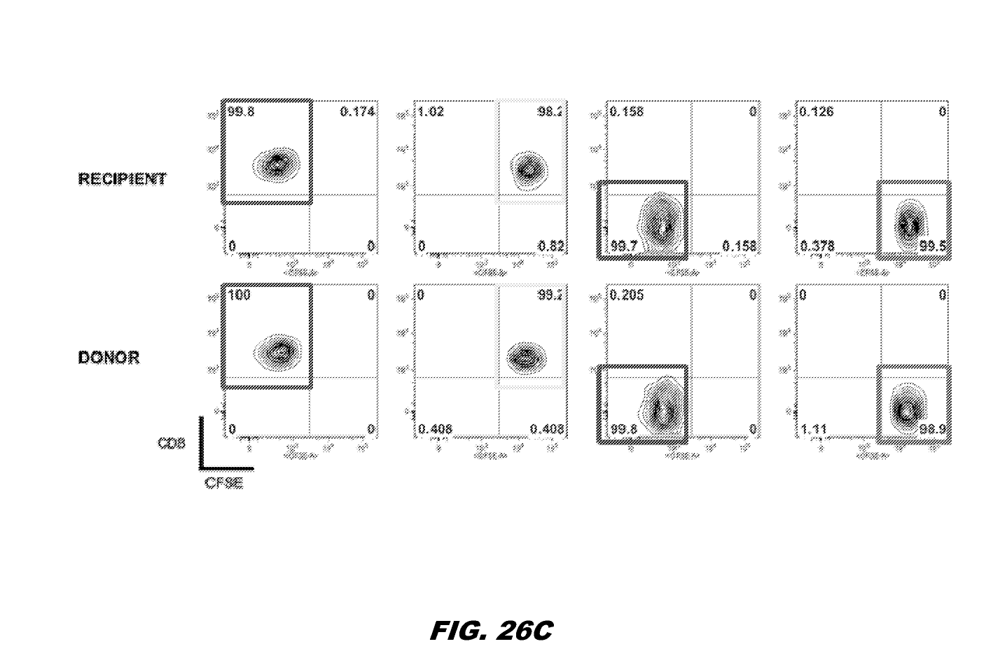

FIGS. 26A-C shows identification and monitoring of Graft v. Host (GvH) and Host v.Graft (HvG)-specific T cell clones with high throughput sequencing.

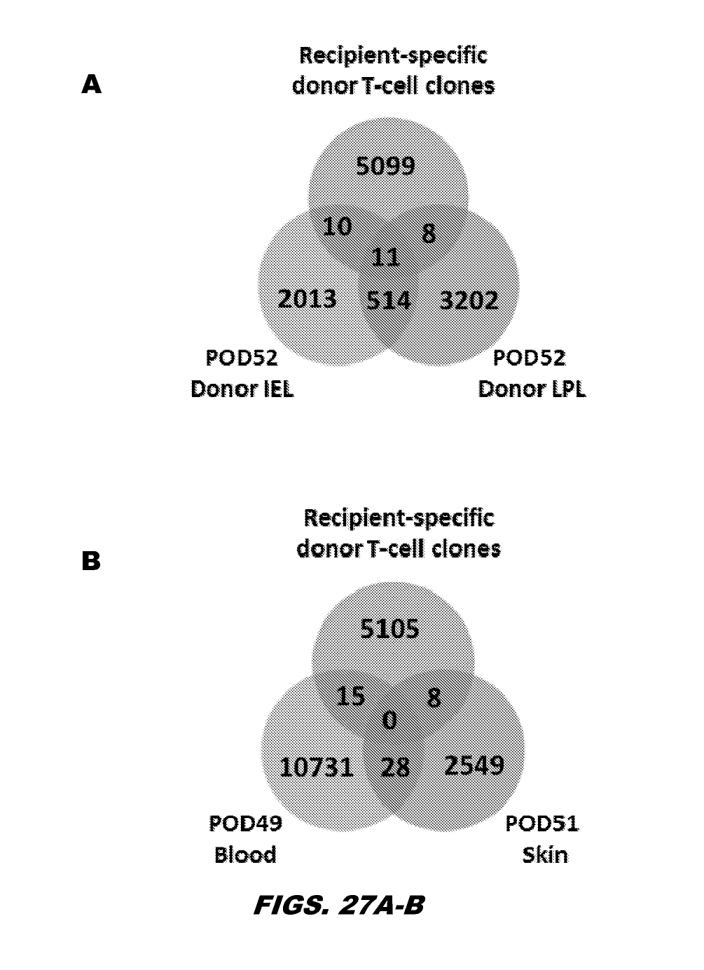

FIGS. 27A-B shows presence of GvH clones in the gut, blood and skin at the time of GVHD. CDR3 overlap (comparing amino acid sequences) between recipient-specific T-cell clones and donor-derived graft-resident T cells, blood and skin at the time of the GVHD. GvH clones were identified in donor-derived sorted IEL and LPL, as well as in the blood and skin at the time of GVHD.

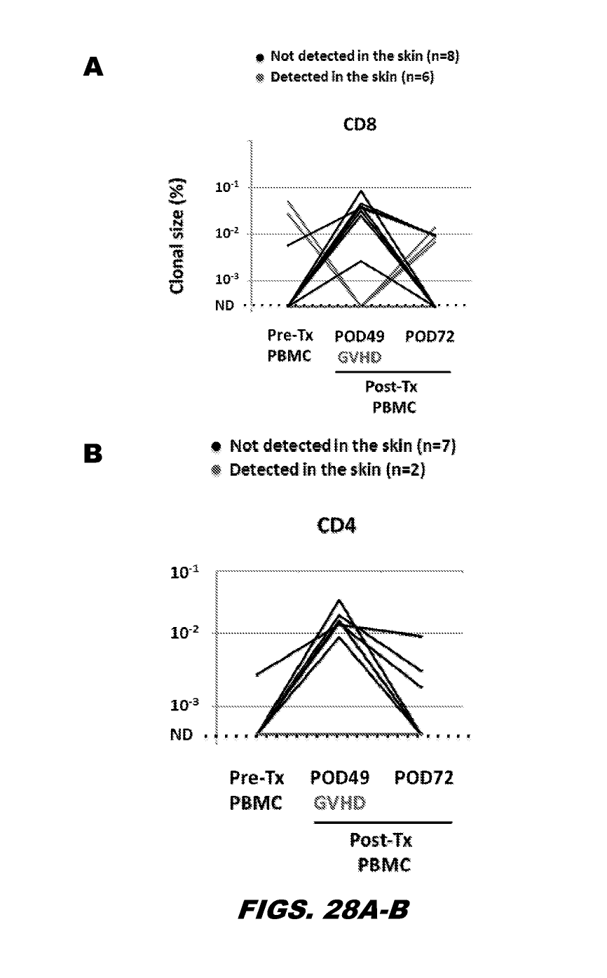

FIGS. 28A-B shows post-transplant trajectories of pre-transplant GvH clones for CD8 and CD4. Pre-transplant GvH clones peaked in the blood at the time of the GVHD, with the exception of those found in the skin, possibly trapped in the target tissue.

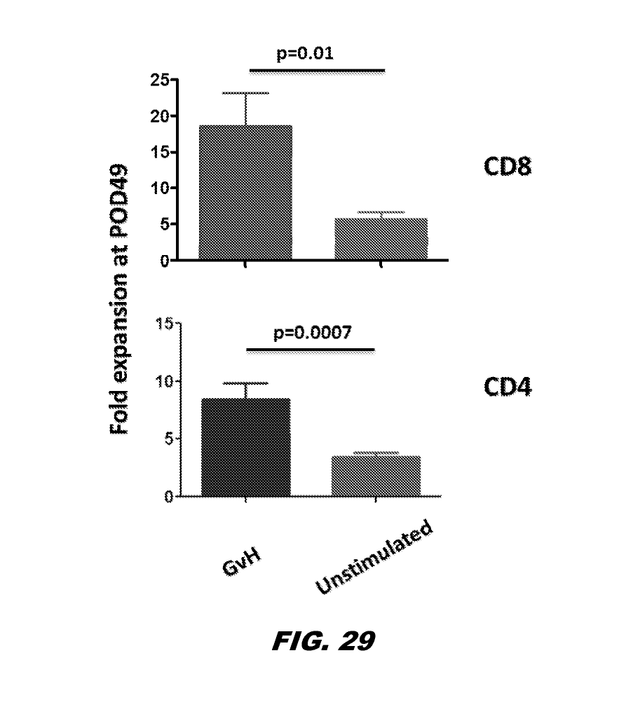

FIG. 29 shows the GvH clones expanded significantly more than unstim clones at the time of GVHD. Frequency fold expansion of pre-transplant GvH and unstim clones detected post-transplant. Although both CD4 and CD8 unstim T cells expanded at the time of GVHD, likely due to lymphopenia-driven proliferation, GvH clones expanded significantly more.

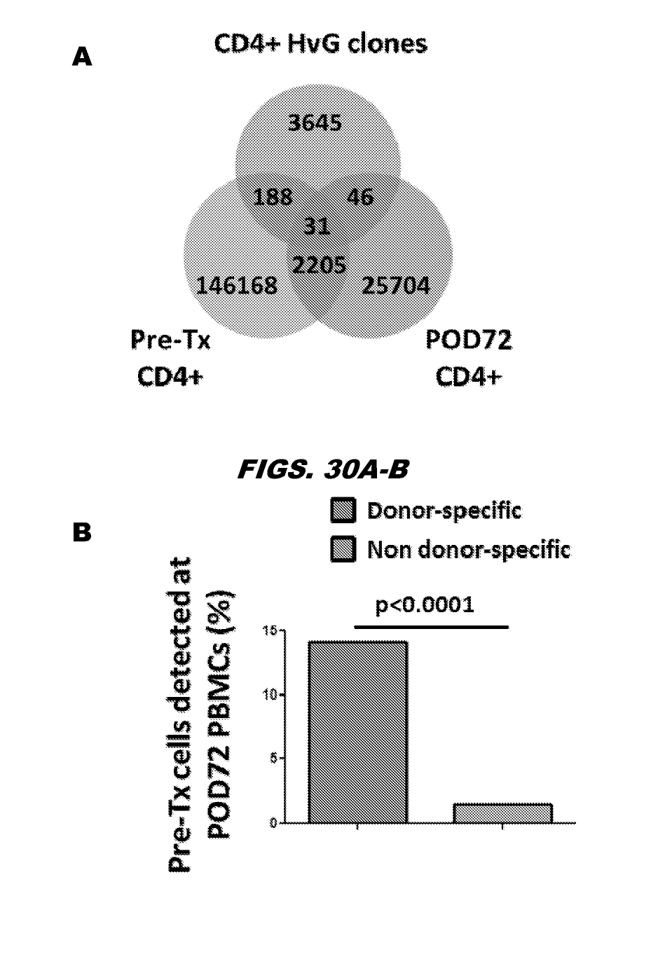

FIGS. 30A-B shows Presence of HvG clones in the blood at the time of the rejection. A significantly greater proportion of pre-Tx donor-specific vs non donor-specific CD4+ clones are detected at POD72 (CDR3 overlap based on amino acid sequences). (31 out of the 219 HvG clones identified before the transplant in unstim samples were still detected in the blood at POD72, versus 2205 out of the 148373 non donor-specific clones (p<0.0001).

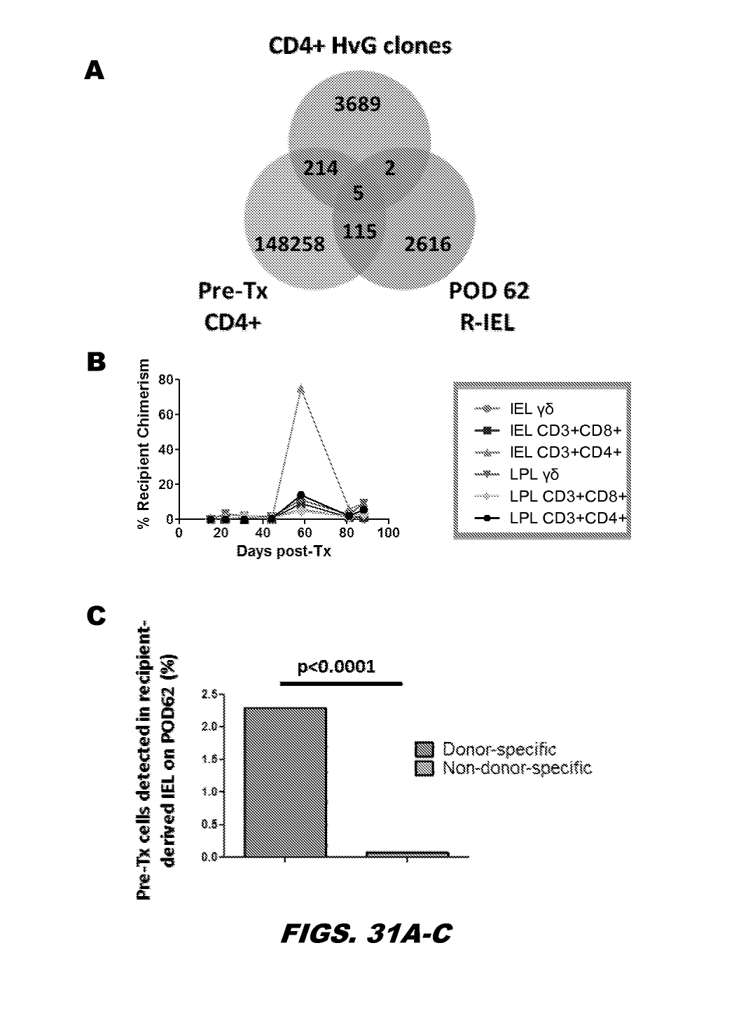

FIG. 31A-C shows presence of HvG CD4+ clones among IEL at the time of the rejection. FIG. 31A: HvG and non-donor-reactive clones were tracked in IEL recipient-derived cell lines expanded from a biopsy performed on POD62 (rejection). A significantly greater proportion of pre-transplant donor-specific vs non donor-specific CD4+ clones are detected in the recipient-derived IEL at the time of rejection (CDR3 overlap based on amino acid sequences). (5 out of the 219 HvG clones detected before the transplant in unstim samples were found in the R-IEL on POD62, versus 115 out of the 148373 non donor-specific clones (p<0.0001).

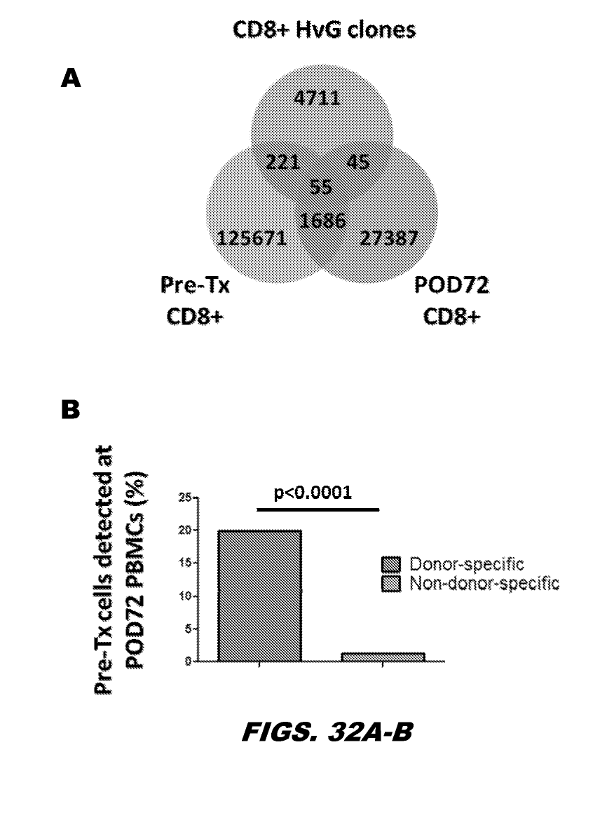

FIGS. 32A-B shows presence of HvG CD8+ clones in the blood at the time of the rejection. A significantly greater proportion of pre-transplant donor-specific vs non donor-specific CD8+ clones are detected at POD72 (CDR3 overlap based on amino acid sequences). (55 out of the 276 HvG clones identified before the transplant in unstim samples were still detected in the blood at POD72, versus 1686 out of the 125671 non donor-specific clones (p<0.0001).

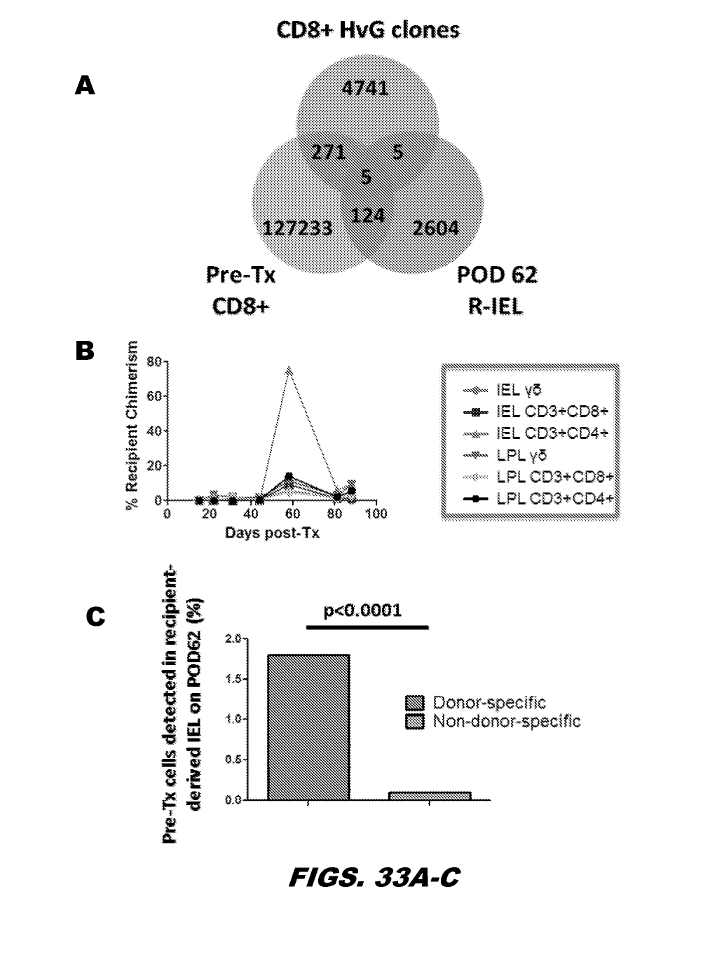

FIGS. 33A-C shows Presence of HvG CD8+ clones among IEL at the time of the rejection. FIG. 33A: HvG and non-donor-reactive clones were tracked in IEL recipient-derived cell lines expanded from a biopsy performed on POD62 (rejection). A significantly greater proportion of pre-transplant donor-specific vs non donor-specific CD8+ clones are detected in the recipient-derived IEL at the time of rejection (CDR3 overlap based on AA sequences). (5 out of the 276 HvG clones detected before the transplant in unstim samples were found in the R-IEL on POD62, versus 124 out of the 127357 non donor-specific clones (p<0.0001).

1-69. (canceled)

70. A method of determining tolerance of a subject to a transplant, comprising:
(a) identifying donor reactive T-cell clones in the subject in an in vitro mixed lymphocyte reaction (MLR);
(b) determining the frequency of said donor reactive T-cell clones identified in step (a) in a pre-transplant sample from the subject;
(c) determining the frequency of said donor reactive T-cell clones identified in step (a) in a post-transplant sample from the subject;
(d) comparing the frequencies of said donor reactive T-cell clones in said pre- and post-transplant samples; and
(e) determining the subject is non-tolerant of the transplant when the frequency of donor reactive T-cell clones in the post-transplant sample is higher than the frequency of donor reactive T-cell clones in said pre-transplant sample, or determining the subject is tolerant of the transplant when the frequency of donor reactive T-cell clones in the post-transplant sample is equal to or less than the frequency of donor reactive T-cell clones in said pre-transplant sample.

71. The method of claim 70, further comprising administering an immunosuppressive therapy to the subject when the subject is determined to be non-tolerant of the transplant.

72. The method of claim 70, further comprising withdrawing the treatment of said subject with an immunosuppressive therapy when the subject is determined to be tolerant of the transplant.

73. The method of claim 71, further comprising determining frequencies of said donor reactive T-cell clones in a sample obtained after said administration.

74. The method of claim 73, further comprising determining a therapeutic effect of said immunosuppressive therapy based on changes in frequencies of said donor reactive T-cell clones.

75. The method of claim 70, wherein said identifying in step (a) comprises:
(a) performing high throughput sequencing of T-cell receptor (TCR) complementarity determining region 3 (CDR3) gene sequences obtained from a first T-cell population from said subject, wherein said first T-cell population comprises an unstimulated sample from said subject;
(b) performing high throughput sequencing of TCR CDR3 region gene sequences obtained from a second T-cell population from said subject, wherein said second T-cell population comprises T-cells that have been cultured in a mixed lymphocyte reaction with peripheral blood mononuclear cells (PBMC) from the donor of said transplant; and
(c) comparing the frequencies of TCR CDR3 region gene sequences of the first T-cell population with the TCR CDR3 region gene sequences of the second T-cell population to identify expanded TCR gene sequences as donor reactive TCR clones.

76. The method of claim 75, wherein the donor reactive T-cells comprise TCR CDR3 gene sequences with a frequency of at least 0.01% in the second T-cell population and/or a 5-fold higher frequency in the second T-cell population compared to the first T-cell population.

77. The method of claim 71, wherein the immunosuppressive therapy is a glucocorticoid, a cytostatic agent, an antibody, an immunophilin modulator, an interferon, plasmapheresis, or a combination thereof.

78. The method of claim 77, wherein the glucocorticoid is methylprednisolone, corticosteroid, prednisone, prednisolone, dexamethasone, or betamethasone.

79. The method of claim 77, wherein the cytostatic is methotrexate, azathioprine, mercaptopurine, dactinomycin, anthracyclines, mitomycin C, bleomycin, mithramycin, mycophenolate mofetil.

80. The method of claim 77, wherein the antibody is a chimeric antibody, a humanized antibody, or a fully human antibody.

81. The method of claim 77, wherein the antibody is thymoglobulin, Atgam, Muromonab-CD3, basiliximab, daclizumab, rituximab, intravenous immunoglobulin.

82. The method of claim 77, wherein the immunophilin modulator is cyclosporine, sirolimus, tacrolimus.

83. The method of claim 77, wherein the interferon is interferon alpha 2a, interferon alpha 2b, interferon beta 1a, interferon beta 1b, interferon gamma 1b.

84. The method of claim 71, wherein the immunosuppressive therapy is methylprednisolone, corticosteroid, thymoglobulin, basiliximab, rituximab, intravenous immunoglobulin, tacrolimus, mycophenolate, plasmapheresis, or a combination thereof.

85. The method of claim 70, wherein the transplant is a heart transplant, a kidney transplant, a liver transplant, a lung transplant, a pancreas transplant, an intestine transplant, a stomach transplant, a testis transplant, a thymus transplant, a hematopoietic cell transplant, or combination thereof.

86. A method of treating transplant rejection in a subject in need thereof comprising:
(a) identifying donor reactive T-cell clones in the subject in an in vitro mixed lymphocyte reaction (MLR);
(b) determining the frequency of donor reactive T-cells in a pre-transplant sample of the subject;
(c) determining the frequency of donor reactive T-cells in a post-transplant sample from the subject; and
(d) administering an immunosuppressive therapy to the subject when the frequency of donor reactive T-cells in the post-transplant sample is equal to or higher than the frequency of donor reactive T-cell receptor beta gene sequences in the pre-transplant sample.

87. The method of claim 86, wherein said identifying in step (a) comprises:
(a) performing high throughput sequencing of T-cell receptor (TCR) complementarity determining region 3 (CDR3) gene sequences obtained from a first T-cell population from said subject, wherein said first T-cell population comprises an unstimulated sample from said subject;
(b) performing high throughput sequencing of TCR CDR3 region gene sequences obtained from a second T-cell population from said subject, wherein said second T-cell population comprises T-cells that have been cultured in a mixed lymphocyte reaction with peripheral blood mononuclear cells (PBMC) from the donor of said transplant; and
(c) comparing the frequencies of TCR CDR3 region gene sequences of the first T-cell population with the TCR CDR3 region gene sequences of the second T-cell population to identify expanded TCR gene sequences as donor reactive TCR clones.

88. The method of claim 87, wherein the donor reactive T-cells comprise TCR CDR3 gene sequences with a frequency of at least 0.01% in the second T-cell population and/or a 5-fold higher frequency in the second T-cell population compared to the first T-cell population.

89. The method of claim 86, wherein the immunosuppressive therapy is a glucocorticoid, a cytostatic agent, an antibody, an immunophilin modulator, an interferon, plasmapheresis, or a combination thereof.
